(19) United States
(12) Patent Application Publication
(10) Pub. No.: US 2012/0225937 A1
(43) Pub. Date: Sep. 6, 2012

(54) CRYSTALLINE FORMS AND PROCESSES FOR THE PREPARATION OF PG12 RECEPTOR AGONISTS

(75) Inventors: Anthony C. Blackburn, San Diego, CA (US); Sagar Raj Shakya, San Diego, CA (US); John A. DeMattei, Berthoud, CO (US); Tsung-Hsun Chuang, San Diego, CA (US)

(73) Assignee: Arena Pharmaceuticals, Inc., San Diego, CA (US)

(21) Appl. No.: 13/497,616

(22) PCT Filed: Sep. 21, 2010
(86) PCT No.: PCT/US10/02574

§ 371 Date: Mar. 22, 2012

Related U.S. Application Data
(60) Provisional application No. 61/277,393, filed on Sep. 23, 2009.

(51) Int. Cl. A61K 31/27; A61P 9/12; A61P 7/02; A61P 9/10; A61P 9/00; A61P 27/06; A61P 25/02; A61P 13/12; A61P 27/02; A61P 29/00; A61P 17/06; A61P 19/02; A61P 37/06; A61P 25/00; A61P 37/00; A61P 1/04; A61P 17/10; A61P 3/10; A61P 11/00; A61P 11/06; A61P 1/00; C07C 309/15

(52) U.S. Cl. 514/488; 560/27

(57) ABSTRACT

The present invention relates to salts of 2-(2-((4-(((4-chlorophenyl)(phenyl)-carbamoyloxy)methyl)cyclohexyl)methoxy)acetamido)ethanesulfonic acid (Compound 1) and crystalline forms, solvates and hydrates thereof. The present invention further relates to processes and intermediates useful in the preparation of Compound I and salts, solvates and hydrates thereof. Crystalline forms, salts, solvates and hydrates of the present invention and pharmaceutical compositions thereof are useful in the treatment of for example, pulmonary arterial hypertension (PAH); platelet aggregation; coronary artery disease; myocardial infarction; transient ischemic attack; angina; stroke; ischemia-reperfusion injury; restenosis; atrial fibrillation; blood clot formation; atherothrombosis; asthma or a symptom thereof; a diabetic-related disorder; glaucoma or other disease of the eye with abnormal intraocular pressure; hypertension; inflammation; psoriasis; psoriatic arthritis; rheumatoid arthritis; Crohn's disease; transplant rejection; multiple sclerosis; systemic lupus erythematosus (SLE); ulcerative colitis; atherosclerosis; acne; type I diabetes; type 2 diabetes; sepsis; and chronic obstructive pulmonary disorder (COPD).

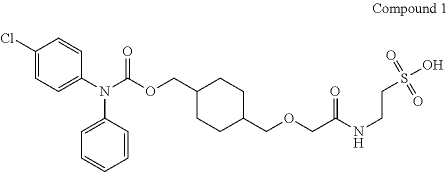

### FIELD OF THE INVENTION

The present invention relates to crystalline forms of 2-(2-((4-(((4-chlorophenyl)(phenyl)carbamoyloxy)methyl)cyclohexyl)methoxy)acetamido)ethanesulfonic acid (Compound I) and salts, solvates and hydrates thereof. The present invention further relates to processes and intermediates useful in the preparation of Compound I and salts, solvates and hydrates thereof. Crystalline forms, salts, solvates, and hydrates of the present invention and pharmaceutical compositions thereof are useful in the treatment of, for example, treatment of: pulmonary arterial hypertension (PAH); idiopathic PAH; familial PAH; PAH associated with: a collagen vascular disease, a congenital heart disease, portal hypertension, HIV infection, ingestion of a drug or toxin, hereditary hemorrhagic telangiectasia, splenectomy, pulmonary veno-occlusive disease (PVOD) or pulmonary capillary hemangiomatosis (PCH); PAH with significant venous or capillary involvement; platelet aggregation; coronary artery disease; myocardial infarction; transient ischemic attack; angina; stroke; ischemia-reperfusion injury; restenosis; atrial fibrillation; blood clot formation in an angioplasty or coronary bypass surgery individual or in an individual suffering from atrial fibrillation; atherothrombosis; asthma or a symptom thereof; a diabetic-related disorder such as diabetic peripheral neuropathy, diabetic nephropathy or diabetic retinopathy; glaucoma or other disease of the eye with abnormal intraocular pressure; hypertension; inflammation; psoriasis; psoriatic arthritis; rheumatoid arthritis; Crohn's disease; transplant rejection; multiple sclerosis; systemic lupus erythematosus (SLE); ulcerative colitis; atherosclerosis; acne; type 1 diabetes; type 2 diabetes; sepsis; and chronic obstructive pulmonary disorder (COPD).

### BACKGROUND OF THE INVENTION

Prostacyclin (PGI2) is a lipid molecule derived from arachidonic acid through the cyclooxygenase pathway. It is a potent vasodilator, antiproliferative, anti-thrombotic and antiplatelet agent that mediates its effects as an agonist of a G protein-coupled receptor (PGI2 receptor; e.g., human PGI2 receptor, GenBank® Accession No. NP_{—}000951 and alleles thereof). It is known that the binding of PGI2 (or other such agonists) to the PGI2 receptor leads to coupling with the Gs protein and increased intracellular cAMP levels. (See, e.g., Zhang et al., Arch. Biochem. Biophys., 2006, 454:80-88.)

Pulmonary arterial hypertension (PAR) is a life-threatening disease characterized by a progressive pulmonary vasculopathy leading to right ventricular hypertrophy. Right heart failure occurs if left untreated. Prostacyclin, which has vasodilatory and antiproliferative effects on the pulmonary vasculature has been found to be low in patients with PAH compared with normal controls. Exogenous administration of prostacyclin or an analog of prostacyclin (i.e., an agonist of the PGI2 receptor) has become an important strategy in the treatment of PAH. (See, e.g., Tuder et al., Am. J. Respir. Crit. Care. Med., 1999, 159:1925-1932; Humbert et al., J. Am. Coll. Cardiol., 2004, 43:13 S-24S; Rosenzweig, Expert Opin. Emerging Drugs, 2006, 11:609-619; McLaughlin et al., Circulation, 2006, 114:1417-1431; Rosenkranz, Clin. Res. Cardiol., 2007, 96:527-541; Driscoll et al., Expert Opin. Pharmacother., 2008, 9:65-81.)

Trepostinil and iloprost are FDA-approved analogs of prostacyclin which, like prostacyclin, are not orally-active. Beraprost is an orally-active analog of prostacyclin approved for the treatment of PAH in Japan, but it has failed registration for the treatment of PAH in Europe and in the US. Of the three FDA-approved drugs, prostacyclin is the best studied in PAH patients. The approximate annual cost of treating PAH with these drugs is $25,000 to $200,000 depending on the dose. At present, many experts consider intravenous prostacyclin to be the most reliable agent for managing the sickest PAH patients. Due to the short half-life of prostacyclin, intravenous treatment is complicated by the need for a continuous infusion. Patients are at risk for potentially fatal rebound pulmonary hypertension if the infusion is abruptly disrupted, as well as significant risk of catheter-related complications including sepsis. (See, e.g., Rosenzweig, Expert Opin. Emerging Drugs, 2006, 11:609-619; Naeije et al., Expert Opin. Pharmacother., 2007, 8:2247-2265; Strauss et al., Clin. Chest. Med., 2007, 28:127-142; Driscoll et al., Expert Opin. Pharmacother., 2008, 9:65-81.)

In view of the growing demand for compounds useful in the treatment of disorders related to the PGI2 receptor, 2-(2-((4-(((4-chlorophenyl)(phenyl)carbamoyloxy)methyl)cyclohexyl)methoxy)acetamido)ethanesulfonic acid has emerged has an important new compound. Accordingly, new and more efficient routes leading to 2-(2-((4-(((4-chlorophenyl)(phenyl)carbamoyloxy)methyl)cyclohexyl)methoxy)acetamido)ethanesulfonic acid, intermediates related thereto, and crystalline forms thereof are needed. The processes and compounds described herein help meet these and other needs.

### SUMMARY OF THE INVENTION

The processes and intermediates of the present invention are useful in preparing 2-(2-((4-(((4-chlorophenyl)(phenyl)carbamoyloxy)methyl)cyclohexyl)methoxy)acetamido)ethanesulfonic acid (Compound I):

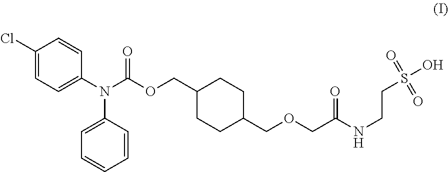

and pharmaceutically acceptable salts, solvates and hydrates thereof.

One aspect of the present invention relates to processes for preparing a compound selected from: 2-(2-((4-(((4-chlorophenyl)(phenyl)carbamoyloxy)methyl)cyclohexyl)methoxy)acetamido)ethanesulfonic acid (Compound I), and pharmaceutically acceptable salts, solvates and hydrates thereof, comprising the following steps:
(a) reacting a compound of (Formula IV):

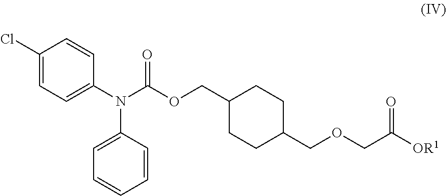

wherein R¹ is selected from: H, an inorganic cation and an organic cation;
with an activating agent to form a compound of Formula

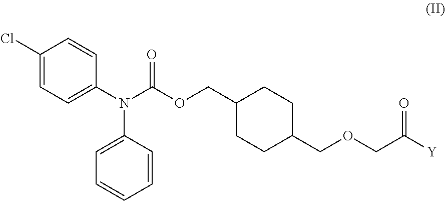

or a salt thereof; wherein Y is an activating group; and
(b) reacting said compound of Formula H, or a salt thereof, with taurine (Compound III):

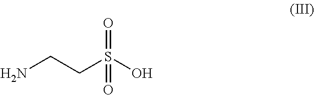

or a salt thereof; to form said Compound I or a pharmaceutically acceptable salt, solvate or hydrate thereof;
   provided that:
(i) if said activating agent is thionyl chloride, then R¹ is other than H; and
(ii) if said activating agent is 1H-benzo[d][1,2,3]triazol-1-ol, and R¹ is sodium, then said reacting of a compound of Formula IV with an activating agent is carried out in the presence of a substituted carbodiimide.

One aspect of the present invention relates to processes for preparing a salt of 2-(2-((4-(((4-chlorophenyl)(phenyl)carbamoyloxy)methyl)cyclohexyl)methoxy)acetamido)ethanesulfonic acid (Compound I):

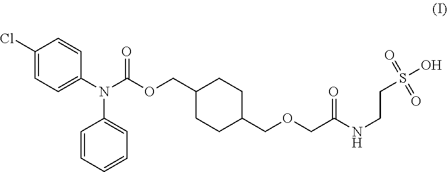

comprising reacting said Compound I with a salt-forming base to form said salt of Compound I;
wherein the anion of said salt of Compound I is 2-(2-((4-(((4-chlorophenyl)(phenyl)carbamoyloxy)methyl)cyclohexyl)methoxy)acetamido)ethanesulfonate;
provided that the cation of said salt of Compound I is other than sodium.

One aspect of the present invention relates to a salt of a compound selected from: 2-(2-((4-(((4-chlorophenyl)(phenyl)carbamoyloxy)methyl)cyclohexyl)methoxy)acetamido)ethanesulfonic acid (Compound I):

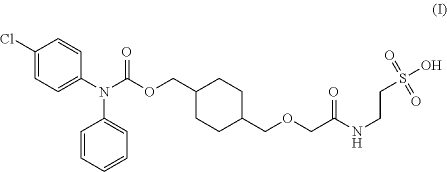

and solvates and hydrates thereof; wherein the anion of said salt of Compound I is 2-(2-((4-(((4-chlorophenyl)(phenyl)carbamoyloxy)methyl)cyclohexyl)methoxy)acetamido)ethanesulfonate;
provided that the cation of said salt of Compound I is other than sodium.

One aspect of the present invention relates to a salt of a compound selected from: 2-(2-((4-(((4-chlorophenyl)(phenyl)carbamoyloxy)methyl)cyclohexyl)methoxy)acetamido)ethanesulfonic acid (Compound I):

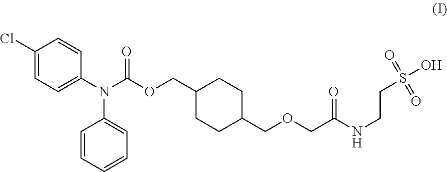

and solvates and hydrates thereof; wherein the anion of said salt of Compound I is 2-(2-((4-(((4-chlorophenyl)(phenyl)carbamoyloxy)methyl)cyclohexyl)methoxy)acetamido)ethanesulfonate;

provided that the cation of said salt of Compound I is other than sodium.

One aspect of the present invention relates to solvates and hydrates of salts of Compound I selected from the following solvates and hydrates:
sodium 2-(2-(((1r,4r)-4-(((4-chlorophenyl)(phenyl)carbamoyloxy)methyl)cyclohexyl)methoxy)acetamido)ethanesulfonate hydrate I;
sodium 2-(2-(((1r,4r)-4-(((4-chlorophenyl)(phenyl)carbamoyloxy)methyl)cyclohexyl)methoxy)acetamido)ethanesulfonate hydrate II;
potassium 2-(2-(((1r,4r)-4-(((4-chlorophenyl)(phenyl)carbamoyloxy)methyl)cyclohexyl)methoxy)acetamido)ethanesulfonate hydrate;
magnesium 2-(2-(((1r,4r)-4-(((4-chlorophenyl)(phenyl)carbamoyloxy)methyl)cyclohexyl)methoxy)acetamido)ethanesulfonate hydrate;
calcium 2-(2-(((1r,4r)-4-(((4-chlorophenyl)(phenyl)carbamoyloxy)methyl)cyclohexyl)methoxy)acetamido)ethanesulfonate hydrate I;
calcium 2-(2-(((1r,4r)-4-(((4-chlorophenyl)(phenyl)carbamoyloxy)methyl)cyclohexyl)methoxy)acetamido)ethanesulfonate hydrate II;
calcium 2-(2-(((1r,4r)-4-(((4-chlorophenyl)(phenyl)carbamoyloxy)methyl)cyclohexyl)methoxy)acetamido)ethanesulfonate hydrate III;
calcium 2-(2-(((1r,4r)-4-(((4-chlorophenyl)(phenyl)carbamoyloxy)methyl)cyclohexyl)methoxy)acetamido)ethanesulfonate hydrate IV;
TRIS 2-(2-(((1r,4r)-4-(((4-chlorophenyl)(phenyl)carbamoyloxy)methyl)cyclohexyl)methoxy)acetamido)ethanesulfonate hydrate; and
L-arginine 2-(2-(((1r,4r)-4-(((4-chlorophenyl)(phenyl)carbamoyloxy)methyl)cyclohexyl)methoxy)acetamido)ethanesulfonate hydrate.

One aspect of the present invention relates to solvates and hydrates of salts of Compound I selected from the following solvates and hydrates:
potassium 2-(2-(((1r,4r)-4-(((4-chlorophenyl)(phenyl)carbamoyloxy)methyl)cyclohexyl)methoxy)acetamido)ethanesulfonate hydrate;
magnesium 2-(2-(((1r,4r)-4-(((4-chlorophenyl)(phenyl)carbamoyloxy)methyl)cyclohexyl)methoxy)acetamido)ethanesulfonate hydrate;
calcium 2-(2-(((1r,4r)-4-(((4-chlorophenyl)(phenyl)carbamoyloxy)methyl)cyclohexyl)methoxy)acetamido)ethanesulfonate hydrate;
TRIS 2-(2-(((1r,4r)-4-(((4-chlorophenyl)(phenyl)carbamoyloxy)methyl)cyclohexyl)methoxy)acetamido)ethanesulfonate hydrate; and
L-arginine 2-(2-(((1r,4r)-4-(((4-chlorophenyl)(phenyl)carbamoyloxy)methyl)cyclohexyl)methoxy)acetamido)ethanesulfonate hydrate.

One aspect of the present invention relates to crystalline forms of a compound selected from: a pharmaceutically acceptable salt of 2-(2-((4-(((4-chlorophenyl)(phenyl)carbamoyloxy)methyl)cyclohexyl)methoxy)acetamido)ethanesulfonic acid (Compound I), and solvates and hydrates thereof.

One aspect of the present invention relates to crystalline forms of a compound selected from: a pharmaceutically acceptable salt of 2-(2-(((1r,4r)-4-(((4-chlorophenyl)(phenyl)carbamoyloxy)methyl)cyclohexyl)methoxy)acetamido)ethanesulfonic acid (Compound Ia), and solvates and hydrates thereof.

One aspect of the present invention relates to crystalline forms of a compound selected from: a pharmaceutically acceptable salt of 2-(2-((4-(((4-chlorophenyl)(phenyl)carbamoyloxy)methyl)cyclohexyl)methoxy)acetamido)ethanesulfonic acid (Compound I), and solvates and hydrates thereof; provided that said compound is other than sodium 2-(2-((4-(((4-chlorophenyl)(phenyl)carbamoyloxy)methyl)cyclohexyl)methoxy)acetamido)ethanesulfonate; and further provided that said compound is other than sodium 2-(2-((4-(((4-chlorophenyl)(phenyl)carbamoyloxy)methyl)cyclohexyl)methoxy)acetamido)ethanesulfonate hydrate.

One aspect of the present invention relates to crystalline forms of a compound selected from: a pharmaceutically acceptable salt of 2-(2-(((1r,4r)-4-(((4-chlorophenyl)(phenyl)carbamoyloxy)methyl)cyclohexyl)methoxy)acetamido)ethanesulfonic acid (Compound Ia), and solvates and hydrates thereof; provided that said compound is other than sodium 2-(2-(((1r,4r)-4-(((4-chlorophenyl)(phenyl)carbamoyloxy)methyl)cyclohexyl)methoxy)acetamido)ethanesulfonate; and further provided that said compound is other than sodium 2-(2-(((1r,4r)-4-(((4-chlorophenyl)(phenyl)carbamoyloxy)methyl)cyclohexyl)methoxy)acetamido)ethanesulfonate hydrate.

One aspect of the present invention relates to pharmaceutical compositions comprising an active pharmaceutical ingredient selected from: a salt as described herein, a solvate or hydrate of a salt as described herein, and a crystalline form as described herein; together with a pharmaceutically acceptable carrier.

One aspect of the present invention relates to methods of preparing pharmaceutical compositions of the present invention, comprising admixing an active pharmaceutical ingredient of the present invention, or a pharmaceutical composition thereof together with a pharmaceutically acceptable carrier.

One aspect of the present invention relates to methods of modulating the activity of a PGI2 receptor by contacting the receptor with an active pharmaceutical ingredient of the present invention, or a pharmaceutical composition thereof.

One aspect of the present invention relates to uses of active pharmaceutical ingredients of the present invention, in the manufacture of a medicament for the treatment of a PGI2 receptor mediated disorder.

One aspect of the present invention relates to active pharmaceutical ingredients of the present invention, for use in a method of treatment of the human or animal body by therapy.

### BRIEF DESCRIPTION OF THE DRAWINGS

FIG. 1 depicts a powder X-ray diffraction pattern (PXRD) for a sample containing a crystalline form of Compound Ia hydrate.

FIG. 2 depicts a differential scanning calorimetry (DSC) thermogram for or a sample containing a crystalline form of Compound Ia hydrate and a thermogravimetric analysis (TGA) thermogram of a sample containing a crystalline form of Compound Ia hydrate.

FIG. 3 depicts a dynamic moisture sorption (DMS) profile for or a sample containing a crystalline form of Compound Ia hydrate.

FIG. 4 depicts a powder X-ray diffraction pattern (PXRD) for a sample containing a crystalline form of Compound Ia sodium salt hydrate I.

FIG. 5 depicts a differential scanning calorimetry (DSC) thermogram for or a sample containing a crystalline form of Compound Ia sodium salt hydrate I and a thermogravimetric analysis (TGA) thermogram of a sample containing a crystalline form of Compound Ia sodium salt hydrate I.

FIG. 6 depicts a powder X-ray diffraction pattern (PXRD) for a sample containing a crystalline form of Compound Ia sodium salt hydrate II.

FIG. 7 depicts a differential scanning calorimetry (DSC) thermogram for or a sample containing a crystalline form of Compound Ia sodium salt hydrate II and a thermogravimetric analysis (TGA) thermogram of a sample containing a crystalline form of Compound Ia sodium salt hydrate II.

FIG. 8 depicts a powder X-ray diffraction pattern (PXRD) for a sample containing a crystalline form of Compound Ia potassium salt hydrate.

FIG. 9 depicts a differential scanning calorimetry (DSC) thermogram for or a sample containing a crystalline form of Compound Ia potassium salt hydrate.

FIG. 10 depicts a thermogravimetric analysis (TGA) thermogram of a sample containing a crystalline form of Compound Ia potassium salt hydrate.

FIG. 11 depicts a dynamic moisture sorption (DMS) profile for or a sample containing a crystalline form of Compound Ia potassium salt hydrate.

FIG. 12 depicts a powder X-ray diffraction pattern (PXRD) for a sample containing a crystalline form of Compound Ia magnesium salt hydrate.

FIG. 13 depicts a thermogravimetric analysis (TGA) thermogram of a sample containing a crystalline form of Compound Ia magnesium salt hydrate.

FIG. 14 depicts a dynamic moisture sorption (DMS) profile for or a sample containing a crystalline form of Compound Ia magnesium salt hydrate.

FIG. 15 depicts a powder X-ray diffraction pattern (PXRD) for a sample containing a crystalline form of a crystalline form of Compound Ia calcium salt hydrate I.

FIG. 16 depicts a differential scanning calorimetry (DSC) thermogram for or a sample containing a crystalline form of Compound Ia calcium salt hydrate I and a thermogravimetric analysis (TGA) thermogram of a sample containing a crystalline form of Compound Ia calcium salt hydrate I.

FIG. 17 depicts a dynamic moisture sorption (DMS) profile for or a sample containing a crystalline form of Compound Ia calcium salt hydrate I.

FIG. 18 depicts a powder X-ray diffraction pattern (PXRD) for a sample containing a crystalline form of a crystalline form of Compound Ia calcium salt hydrate II.

FIG. 19 depicts a differential scanning calorimetry (DSC) thermogram for or a sample containing a crystalline form of Compound Ia calcium salt hydrate II and a thermogravimetric analysis (TGA) thermogram of a sample containing a crystalline form of Compound Ia calcium salt hydrate II.

FIG. 20 depicts a powder X-ray diffraction pattern (PXRD) for a sample containing a crystalline form of a crystalline form of Compound Ia calcium salt hydrate M.

FIG. 21 depicts a differential scanning calorimetry (DSC) thermogram for or a sample containing a crystalline form of Compound Ia calcium salt hydrate III and a thermogravimetric analysis (TGA) thermogram of a sample containing a crystalline form of Compound Ia calcium salt hydrate M.

FIG. 22 depicts a powder X-ray diffraction pattern (PXRD) for a sample containing a crystalline form of a crystalline form of Compound Ia calcium salt hydrate IV.

FIG. 23 depicts a differential scanning calorimetry (DSC) thermogram for or a sample containing a crystalline form of Compound Ia calcium salt hydrate IV and a thermogravimetric analysis (TGA) thermogram of a sample containing a crystalline form of Compound Ia calcium salt hydrate IV.

FIG. 24 depicts a powder X-ray diffraction pattern (PXRD) for a sample containing a crystalline form of a crystalline form of Compound Ia TRIS salt.

FIG. 25 depicts a differential scanning calorimetry (DSC) thermogram for or a sample containing a crystalline form of Compound Ia TRIS salt and a thermogravimetric analysis (TGA) thermogram of a sample containing a crystalline form of Compound Ia TRIS salt.

FIG. 26 depicts a dynamic moisture sorption (DMS) profile for or a sample containing a crystalline form of Compound Ia TRIS salt.

FIG. 27 depicts a powder X-ray diffraction pattern (PXRD) for a sample containing a crystalline form of a crystalline form of Compound Ia TRIS salt hydrate.

FIG. 28 depicts a differential scanning calorimetry (DSC) thermogram for or a sample containing a crystalline form of Compound Ia TRIS salt hydrate and a thermogravimetric analysis (TGA) thermogram of a sample containing a crystalline form of Compound Ia TRIS salt hydrate.

FIG. 29 depicts a powder X-ray diffraction pattern (PXRD) for a sample containing a crystalline form of a crystalline form of Compound Ia L-arginine salt hydrate.

FIG. 30 depicts a differential scanning calorimetry (DSC) thermogram for or a sample containing a crystalline form of Compound Ia L-arginine salt hydrate and a thermogravimetric analysis (TGA) thermogram of a sample containing a crystalline form of Compound Ia L-arginine salt hydrate.

FIG. 31 depicts a dynamic moisture sorption (DMS) profile for or a sample containing a crystalline form of Compound Ia L-arginine salt hydrate.

FIG. 32 depicts an overlay of two powder X-ray diffraction patterns. Sample A comprised a crystalline form of Compound Ia potassium salt hydrate containing about 6% water by TGA. Further drying produced Compound Ia potassium salt hydrate Sample B containing just 3.1% water by TGA. The shift of certain peaks to higher 2θ-values in the drier sample can be interpreted as a contracted or partially collapsed unit cell in the lattice, a phenomenon known for dehydrated or partially dehydrated channel hydrates.

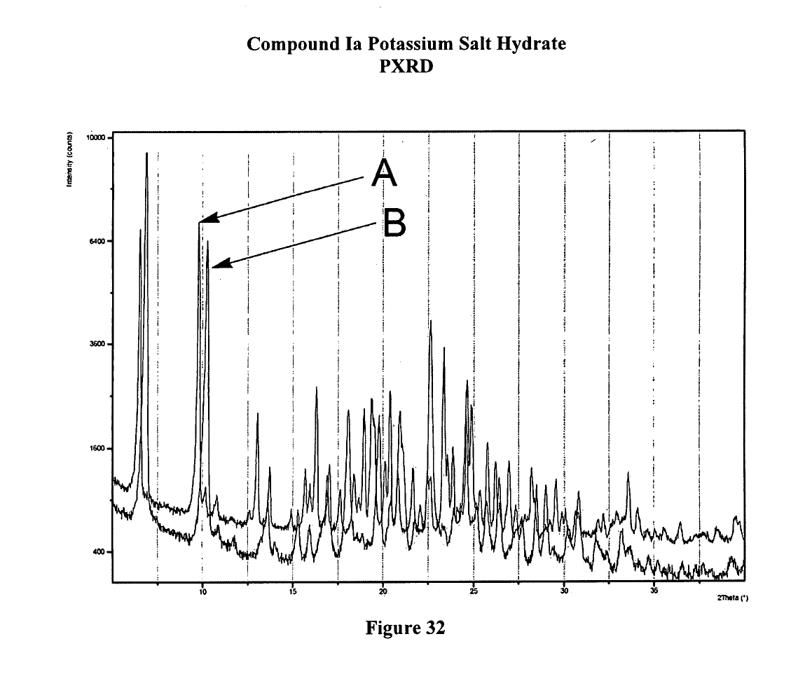

### DETAILED DESCRIPTION

### Definitions

For clarity and consistency, the following definitions will be used throughout this patent document.

The term “agonists” is intended to mean moieties that interact and activate a receptor, such as the receptor, and initiate a physiological or pharmacological response characteristic of that receptor, for example, moieties that activate the intracellular response upon binding to the receptor, or enhance GTP binding to membranes.

The term “hydrate” as used herein means a compound, including but not limited to a pharmaceutically acceptable salt of a compound, that further includes a stoichiometric or non-stoichiometric amount of water bound by non-covalent intermolecular forces.

The term “individual” is intended to mean any animal, including mammals, preferably mice, rats, other rodents, rabbits, dogs, cats, swine, cattle, sheep, horses, or primates and most preferably humans.

The term “pharmaceutical composition” is intended to mean a composition comprising at least one active ingredient; including but not limited to Compound I and pharmaceutically acceptable salts, solvates, and hydrates thereof, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “solvate” as used herein means a compound, including but not limited to a pharmaceutically acceptable salt of a compound, that further includes a stoichiometric or non-stoichiometric amount of a solvent bound by non-covalent intermolecular forces. Preferred solvents are volatile, non-toxic, and/or acceptable for administration to humans in trace amounts.

The term “substituted” indicates that at least one hydrogen atom of a chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

The term “treatment” as used herein includes one or more of the following:

(1) prevention of a disease, for example, prevention of a disease, condition or disorder in an individual that may be predisposed to the disease, condition or disorder but does not yet experience or display the pathology or symptomatology of the disease;

(2) inhibition of a disease, for example, inhibition of a disease, condition or disorder in an individual that is experiencing or displaying the pathology or symptomatology of the disease, condition or disorder (i.e., arresting further development of the pathology and/or symptomatology); and

(3) amelioration of a disease, for example, amelioration of a disease, condition or disorder in an individual that is experiencing or displaying the pathology or symptomatology of the disease, condition or disorder (i.e., reversing the pathology and/or symptomatology).

Whether an individual is in need of treatment is a judgment made by a caregiver (e.g. nurse practitioner, physician, physician assistant, nurse, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by Compound I and pharmaceutically acceptable salts, solvates and hydrates thereof. Accordingly, Compound I and pharmaceutically acceptable salts, solvates and hydrates thereof can be used in a protective or preventive manner; or Compound I and pharmaceutically acceptable salts, solvates and hydrates thereof can be used to alleviate, inhibit or ameliorate a disease, condition or disorder.

### Chemical Group, Moiety or Radical

The term “C₁-C₄ alkoxy” is intended to mean a C₁-C₄ alkyl radical, as defined herein, attached directly to an oxygen atom. Some embodiments are 1 to 4 carbons. Some embodiments are 1 to 3 carbons. Some embodiments are 1 or 2 carbons. Examples include methoxy, ethoxy, n-propoxy, isopropoxy, n-butoxy, t-butoxy, isobutoxy, sec-butoxy and the like.

The term “C₁-C₄ alkyl” is intended to mean a straight or branched carbon radical containing 1 to 4 carbons. Some embodiments are 1 to 4 carbons. Some embodiments are 1 to 3 carbons. Some embodiments are 1 or 2 carbons. Some embodiments are 1 carbon. Examples of an alkyl include, but are not limited to, methyl, ethyl, n-propyl, isopropyl, n-butyl, sec-butyl, isobutyl, t-butyl, and the like.

The term “carbamimidoyloxy” is intended to mean the following group:

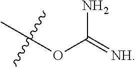

The term “C₂-C₈ dialkylamino” is intended to mean an amino substituted with two of the same or different C₁-C₄ alkyl radicals wherein alkyl radical has the same definition as described herein. The examples include, but are not limited to, dimethylamino, methylethylamino, diethylamino, methylpropylamino, methylisopropylamino, ethylpropylamino, ethylisopropylamino, dipropylamino, propylisopropylamino and the like.

The term “halogen” or “halo” is intended to mean to a fluoro, chloro, bromo or iodo group.

The term “heteroaryl” is intended to mean a ring system containing 5 to 14 ring atoms, that may contain a single ring, two fused rings or three fused rings, and wherein at least one ring is aromatic and at least one ring atom is a heteroatom selected from, for example: O, S and N, wherein N is optionally substituted with H, C₁-C₄ acyl or C₁-C₄ alkyl. Some embodiments contain 5 to 6 ring atoms for example furanyl, thienyl, pyrrolyl, imidazolyl, oxazolyl, thiazolyl, isoxazolyl, pyrazolyl, isothiazolyl, oxadiazolyl, triazolyl, tetrazolyl, thiadiazolyl, pyridinyl, pyrazinyl, pyrimidinyl, pyridazinyl, triazinyl, and the like. Some embodiments contain 8 to 14 ring atoms for example quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl, phenazinyl, phenothiazinyl, phenoxazinyl, benzoxazolyl, benzothiazolyl, 1H-benzimidazolyl, 1H-benzo[d][1,2,3]triazol-1-yl, imidazopyridinyl, benzothienyl, benzofuranyl, isobenzofuran, 2,3-dihydrobenzofuranyl, 4H-benzo[1,3]dioxinyl, 3,4-dihydro-1H-isoquinolinyl, 1,4,6,7-tetrahydro-imidazo[4,5-c]pyridinyl, 7,8-dihydro-5H-[1,6]naphthyridinyl, 5,6-dihydro-8H-[1,2,4]triazolo[4,3-a]pyrazinyl, benzo[1,3]dioxolyl, pyrazolo[1,5-a]pyrimidinyl, 1,2,3,4-tetrahydroquinolinyl, and the like.

The term “heteroaryloxy” is intended to mean a radical comprising a heteroaryl group, attached to an oxygen, wherein heteroaryl has the same definition as found herein.

The term “TRIS” is intended to mean tris(hydroxymethyl)aminomethane.

### Processes of the Invention

The present invention is directed, inter alia, to processes and intermediates useful in the preparation of 2-(2-((4-(((4-chlorophenyl)(phenyl)carbamoyloxy)methyl)cyclohexyl)methoxy)acetamido)ethanesulfonic acid, a PGI2 receptor modulator that is useful in the treatment of pulmonary arterial hypertension (PAH); platelet aggregation; coronary artery disease; myocardial infarction; transient ischemic attack; angina; stroke; ischemia-reperfusion injury; restenosis; atrial fibrillation; blood clot formation; atherothrombosis; asthma or a symptom thereof; a diabetic-related disorder; glaucoma or other disease of the eye with abnormal intraocular pressure; hypertension; inflammation; psoriasis; psoriatic arthritis; rheumatoid arthritis; Crohn's disease; transplant rejection; multiple sclerosis; systemic lupus erythematosus (SLE); ulcerative colitis; atherosclerosis; acne; type 1 diabetes; type 2 diabetes; sepsis; and chronic obstructive pulmonary disorder (COPD).

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

In some embodiments, preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 3^{rd} Ed., Wiley & Sons, 1999, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

Suitable solvents can include halogenated solvents such as: carbon tetrachloride, bromodichloromethane, dibromochloromethane, bromoform, chloroform, bromochloromethane, dibromomethane, butyl chloride, dichloromethane, tetrachloroethylene, trichloroethylene, 1,1,1-trichloroethane, 1,1,2-trichloroethane, 1,1-dichloroethane, 2-chloropropane, hexafluorobenzene, 1,2,4-trichlorobenzene, 1,2-dichlorobenzene, 1,3-dichlorobenzene, 1,4-dichlorobenzene, chlorobenzene, fluorobenzene, fluorotrichloromethane, chlorotrifluoromethane, bromotrifluoromethane, carbon tetrafluoride, dichlorofluoromethane, chlorodifluoromethane, trifluoromethane, 1,2-dichlorotetrafluorethane and hexafluoroethane.

Suitable solvents can include ether solvents, such as: dimethoxymethane, tetrahydrofuran, 2-methyltetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, and t-butyl methyl ether.

Suitable solvents can include protic solvents, such as: water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, isobutyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, and glycerol.

Suitable solvents can include aprotic solvents, such as: benzene, cyclohexane, pentane, hexane, toluene, cycloheptane, methylcyclohexane, heptane, ethylbenzene, o, m-, or p-xylene, octane, indane, nonane, naphthalene, tetrahydrofuran, acetonitrile, dimethyl sulfoxide, propionitrile, ethyl formate, methyl acetate, hexachloroacetone, acetone, ethyl methyl ketone, ethyl acetate, isopropyl acetate, sulfolane, 1,3-dimethyl-3,4,5,6-tetrahydro-2(1H)-pyrimidinone, 1,3-dimethyl-2-imidazolidinone, N-methylpyrrolidinone, tetramethylurea, nitromethane, and nitrobenzene, and amides, including but not limited to, N,N-dimethylformamide, N,N-dimethylacetamide, formamide, N-methylacetamide, N-methylformamide, N,N-dimethylpropionamide, and hexamethylphosphoramide. It is understood by a person of ordinary skill in the art that that the term amide refers to the following formula:

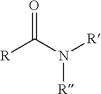

wherein R, R′, and R″ may be the same or different. In some embodiments, R, R′, and R″ are independently selected from H and C₁-C₆ alkyl. In some embodiments, R, R′, and R″ are independently selected from H and C₁-C₄ alkyl. In some embodiments, R, R′, and R″ are independently selected from H and C₁-C₂ alkyl.

Certain chlorination reactions described herein may be performed in the presence of certain amides such as, without limitation, N,N-dimethylformamide and N,N-dimethylacetamide. It is understood by a person of ordinary skill in the art, that although amides can function as solvents, the role of the amide in certain chlorination reactions described herein may be primarily that of a catalyst. It is further understood by a person of ordinary skill in the art, that when chlorination reactions are described herein as being performed “in the substantial absence of solvent” such reactions are intended to include those that are performed in the presence of an amide catalyst, but in the substantial absence of a further solvent.

Supercritical carbon dioxide can also be used as a solvent.

The reactions of the processes described herein can be carried out at appropriate temperatures which can be readily determined by one skilled in the art. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures).

The reactions of the processes described herein can be carried out in air or under an inert atmosphere. Typically, reactions containing reagents or products that are substantially reactive with air can be carried out using air-sensitive synthetic techniques that are well known to one skilled in the art.

In some embodiments, processes of the present invention involve the activation of a carboxylic acid or a salt thereof, e.g. a compound of Formula IV, with a suitable activating agent. Suitable activating agents include, without limitation, thionyl chloride, oxalyl chloride, thionyl bromide, oxalyl bromide, N1-((ethylimino)methylene)-N3,N3-dimethylpropane-1,3-diamine hydrochloride (EDC), 1-hydroxy-1,2,3-benzotriazole (HOBt), 7-aza-1-hydroxy-1,2,3-benzotriazole (HOAt), 2-chloro-4,6-dimethoxy-1,3,5-triazine (CDMT), 3-hydroxybenzo[d][1,2,3]triazin-4(3H)-one (HOOBt), 7-azabenzotriazol-1-yloxy-tris-(pyrrolidino)phosphonium hexafluorophosphate (PyAOP), N-hydroxysuccinimide (HOSu), 3-sulfo-1-hydroxysuccinimide, 2-(1H-7-azabenzotriazol-1-yl)-1,1,3,3-tetramethyl uronium hexafluorophosphate (HATU), 2-(1H-benzotriazol-1-yl)-1,1,3,3-tetramethyluronium hexafluorophosphate (HBTU), 2-(6-chloro-1H-benzotriazole-1-yl)-1,1,3,3-tetramethylaminium hexafluorophosphate (HCTU), dicyclohexylcarbodiimide (DCC), dicyclohexylcarbodiimide (DIC), (1H-1,2,3-benzotriazol-1-yloxy)-tris(dimethylamino)-phosphonium hexafluorophosphate (BOP), (1H-1,2,3-benzotriazol-1-yloxy)-tris(pyrrolidino)-phosphonium hexafluorophosphate (PyBOP), bromo-tris(dimethylamino)-phosphonium hexafluorophosphate (BrOP), bromo-tris(pyrrolidino)-phosphonium hexafluorophosphate (PyBrOP), bis(2-oxooxazolidin-3-yl)phosphinic chloride (BOPCl), 2-(1H-benzotriazole-1-yl)-1,1,3,3-tetramethyluronium tetrafluoroborate (TBTU), and tetramethylfluoroformamidinium hexafluorophosphate (TFFH), and the like. The product of the activation of a carboxylic acid or a salt thereof, e.g. a compound of Formula IV, with a suitable activating agent contains a carboxylic activating group, e.g., in a compound of Formula II, the variable Y. Suitable carboxylic activating groups include, without limitation, 1H-benzo[d][1,2,3]triazol-1-yloxy, 2,5-dioxo-3, sulfopyrrolidin-1-yloxy, 2,5-dioxopyrrolidin-1-yloxy, 3H-[1,2,3]triazolo[4,5-b]pyridin-3-yloxy, 4,6-dimethoxy-1,3,5-triazin-2-yloxy, 4-oxobenzo[d][1,2,3]triazin-3(4H)-yloxy, 6-chloro-1H-benzo[d][1,2,3]triazol-1-yloxy, bis(2-oxooxazolidin-3-yl)phosphoryloxy, bis(dimethylamino)methoxy, bromo, chloro, diisopropylcarbamimidoyloxy, iodo, N-(3-(dimethylamino)propyl)-N′-ethylcarbamimidoyloxy, N,N-dicyclohexylcarbamimidoyloxy, tris(dimethylamino)phosphoniooxy, and tris(pyrrolidino)phosphoniooxy, and the like.

In some embodiments, preparation of compounds can involve the addition of acids or bases to effect, for example, catalysis of a desired reaction or formation of salt forms such as acid addition salts.

Example acids can be inorganic or organic acids. Inorganic acids include hydrochloric acid, hydrobromic acid, sulfuric acid, phosphoric acid, and nitric acid. Organic acids include formic acid, acetic acid, propionic acid, butanoic acid, methanesulfonic acid, p-toluene sulfonic acid, benzenesulfonic acid, propiolic acid, butyric acid, 2-butynoic acid, vinyl acetic acid, pentanoic acid, hexanoic acid, heptanoic acid, octanoic acid, nonanoic acid and decanoic acid.

Example bases include lithium hydroxide, sodium hydroxide, potassium hydroxide, lithium carbonate, sodium carbonate, and potassium carbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include sodium and potassium salts of methyl, ethyl, n-propyl, isopropyl, n-butyl, t-butyl, trimethylsilyl and cyclohexyl substituted amides. Some example organic bases include, but are not limited to, arginine, triethylamine, tributylamine, 4-methylmorpholine, 4-dimethylaminopyridine, diisopropylethylamine, and tris(hydroxymethyl)aminomethane and the like.

The compounds described herein can be asymmetric (e.g., having one or more stereocenters). All stereoisomers, such as enantiomers and diastereomers, are intended unless otherwise indicated. Salts of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis.

The processes described herein can be stereoselective such that any given reaction starting with one or more chiral reagents enriched in one stereoisomer forms a product that is also enriched in one stereoisomer. The reaction can be conducted such that the product of the reaction substantially retains one or more chiral centers present in the starting materials. The reaction can also be conducted such that the product of the reaction contains a chiral center that is substantially inverted relative to a corresponding chiral center present in the starting materials.

Resolution of racemic mixtures of compounds can be carried out by any of numerous methods known in the art. An example method includes fractional recrystallization (for example, diastereomeric salt resolution) using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of β-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

The compounds described herein and salts thereof can also include all isotopes of atoms occurring in the intermediates or final compounds or salts thereof. Isotopes include those atoms having the same atomic number but different mass numbers. For example, isotopes of hydrogen include tritium and deuterium.

The compounds described herein and salts thereof can also include tautomeric forms, such as keto-enol tautomers. Tautomeric forms can be in equilibrium or sterically locked into one form by appropriate substitution.

Upon carrying out preparation of compounds according to the processes described herein, the usual isolation and purification operations such as concentration, filtration, extraction, solid-phase extraction, recrystallization, chromatography, and the like may be used, to isolate the desired products.

Example processes and intermediates of the present invention are provided below in Scheme I.

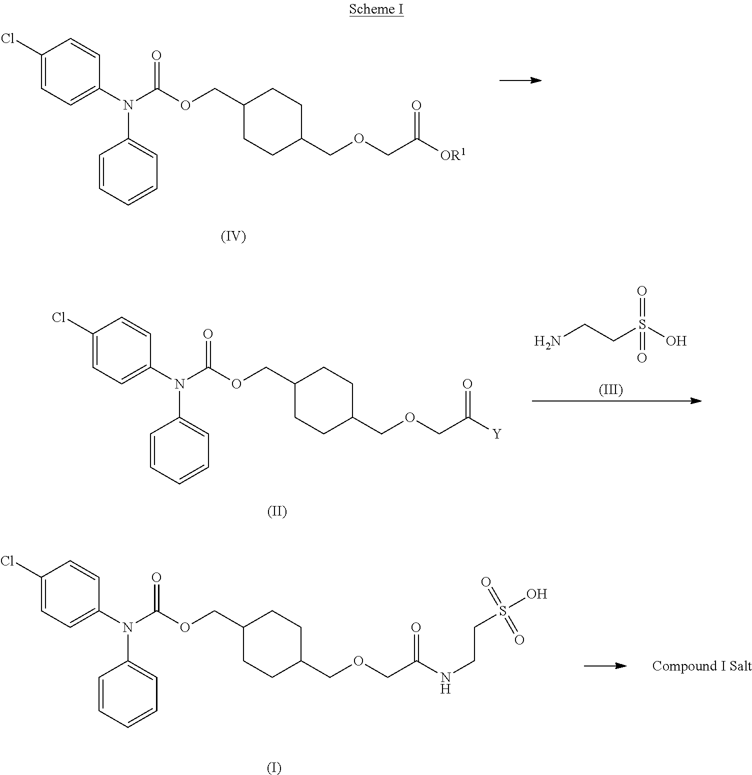

One aspect of the present invention pertains to processes, such as those exemplified by Scheme I (supra), wherein R¹ and Y have the same definitions as described herein, supra and infra.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

### Activation Step

One aspect of the present invention pertains to processes for preparing a compound of Formula II:

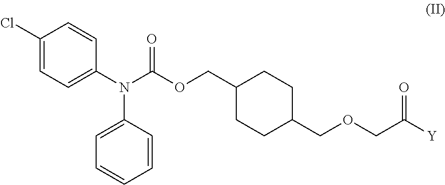

or a salt thereof; wherein Y is an activating group; comprising reacting 2-((4-(((4-chlorophenyl)(phenyl)carbamoyloxy)methyl)cyclohexyl)methoxy)acetic acid (Formula IV):

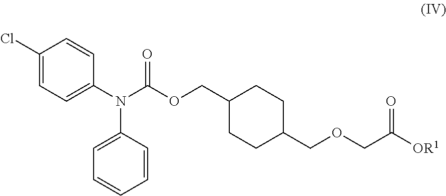

wherein R¹ is selected from: H, an inorganic cation and an organic cation; with an activating agent to form said compound of Formula II, or a salt thereof.

In some embodiments, the activating agent is selected from: thionyl chloride, oxalyl chloride, thionyl bromide, oxalyl bromide, N1-((ethylimino)methylene)-N3,N3-dimethylpropane-1,3-diamine hydrochloride, 1-hydroxy-1,2,3-benzotriazole, 7-aza-1-hydroxy-1,2,3-benzotriazole, 2-chloro-4,6-dimethoxy-1,3,5-triazine, 3-hydroxybenzo[d][1,2,3]triazin-4(3H)-one, 7-azabenzotriazol-1-yloxy-tris-(pyrrolidino)phosphonium hexafluorophosphate, N-hy droxysuccinimide, 3-sulfo-1-hydroxysuccinimide, 2-(1H-7-azabenzotriazol-1-yl)-1,1,3,3-tetramethyl uronium hexafluorophosphate, 2-(1H-benzotriazol-1-yl)-1,1,3,3-tetramethyluronium hexafluorophosphate, 2-(6-chloro-1H-benzotriazole-1-yl)-1,1,3,3-tetramethylaminium hexafluorophosphate, dicyclohexylcarbodiimide, dicyclohexylcarbodiimide, (1H-1,2,3-benzotriazol-1-yloxy)-tris(dimethylamino)-phosphonium hexafluorophosphate, (1H-1,2,3-benzotriazol-1-yloxy)-tris(pyrrolidino)-phosphonium hexafluorophosphate, bromo-tris(dimethylamino)-phosphonium hexafluorophosphate, bromo-tris(pyrrolidino)-phosphonium hexafluorophosphate, bis(2-oxooxazolidin-3-yl)phosphinic chloride, 2-(1H-benzotriazole-1-yl)-1,1,3,3-tetramethyluronium tetrafluoroborate, and tetramethylfluoroformamidinium hexafluorophosphate.

In some embodiments, Y is selected from: 1H-benzo[d][1,2,3]triazol-1-yloxy, 2,5-dioxo-3, sulfopyrrolidin-1-yloxy, 2,5-dioxopyrrolidin-1-yloxy, 3H-[1,2,3]triazolo[4,5-b]pyridin-3-yloxy, 4,6-dimethoxy-1,3,5-triazin-2-yloxy, 4-oxobenzo[d][1,2,3]triazin-3(4H)-yloxy, 6-chloro-1H-benzo[d][1,2,3]triazol-1-yloxy, bis(2-oxooxazolidin-3-yl)phosphoryloxy, bis(dimethylamino)methoxy, bromo, chloro, diisopropylcarbamimidoyloxy, iodo, N-(3-(dimethylamino)propyl)-N′-ethylcarbamimidoyloxy, NX-dicyclohexylcarbamimidoyloxy, tris(dimethylamino)phosphoniooxy, and tris(pyrrolidino)phosphoniooxy.

One aspect of the present invention pertains to processes for preparing a compound of Formula II:

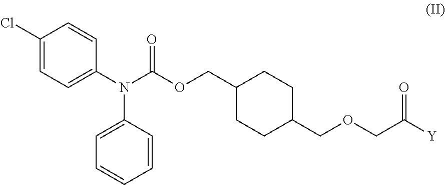

or a salt thereof; wherein Y is an activating group; comprising reacting 2-((4-(((4-chlorophenyl)(phenyl)carbamoyloxy)methyl)cyclohexyl)methoxy)acetic acid (Formula IV):

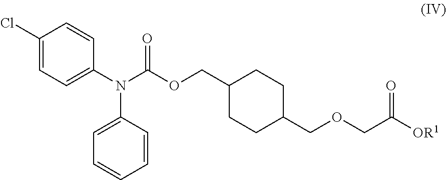

wherein R¹ is selected from: H, an inorganic cation and an organic cation; with an activating agent to form said compound of Formula II, or a salt thereof;

provided that:
(i) If said activating agent is thionyl chloride, then R¹ is other than H; and
(ii) If said activating agent is 1H-benzo[d][1,2,3]triazol-1-ol, and R¹ is sodium, then said reacting of a compound of Formula IV with an activating agent is carried out in the presence of a substituted carbodiimide.

One aspect of the present invention pertains to processes for preparing a compound of Formula II:

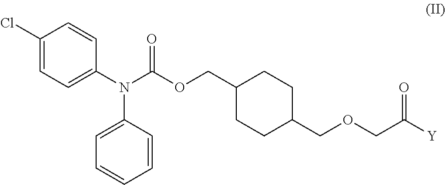

or a salt thereof; wherein Y is selected from: halogen; heteroaryloxy, and carbamimidoyloxy;
wherein: heteroaryloxy is optionally substituted with one or more C₁-C₄ alkoxy substituents; and
carbamimidoyloxy is optionally substituted with one or more C₁-C₆ alkyl substituents; wherein
each C₁-C₆ alkyl is optionally substituted with one or more C₂-C₈ dialkylamino substituents;
comprising reacting 2-((4-(((4-chlorophenyl)(phenyl)carbamoyloxy)methyl)cyclohexyl)methoxy)acetic acid (Formula IV):

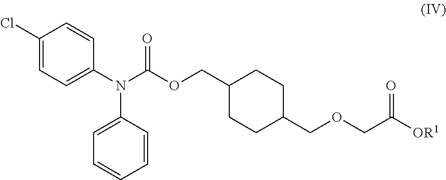

wherein R¹ is selected from: H and metal; with an activating agent to form said compound of Formula II, or a salt thereof;

provided that:
(i) If said activating agent is thionyl chloride, then R¹ is other than H; and
(ii) If said activating agent is 1H-benzo[d][1,2,3]triazol-1-01, and R¹ is sodium, then said reacting of a compound of Formula IV with an activating agent is carried out in the presence of a substituted carbodiimide.

In some embodiments, R¹ is selected from: H and metal.

In some embodiments, R¹ is selected from: H and an alkali metal.

In some embodiments, R¹ is selected from: H, sodium, and potassium.

In some embodiments, R¹ is H.

In some embodiments, R¹ is sodium.

In some embodiments, R¹ is potassium.

In some embodiments, Y is selected from: halogen; heteroaryloxy, and carbamimidoyloxy; wherein: heteroaryloxy is optionally substituted with one or more C₁-C₄ alkoxy substituents; and carbamimidoyloxy is optionally substituted with one or more C₁-C₄ alkyl substituents; wherein each C₁-C₄ alkyl is optionally substituted with one or more C₂-C₈ dialkylamino substituents.

In some embodiments, Y is selected from: chloro, 1H-benzo[d][1,2,3]triazol-1-yloxy, 4,6-dimethoxy-1,3,5-triazin-2-yloxy, and N-(3-(dimethylamino)propyl)-N′-ethylcarbamimidoyloxy.

In some embodiments, Y is selected from: chloro and 1H-benzo[d][1,2,3]triazol-1-yloxy, and 4,6-dimethoxy-1,3,5-triazin-2-yloxy.

In some embodiments, Y is chloro.

In some embodiments, Y is 1H-benzo[d][1,2,3]triazol-1-yloxy.

In some embodiments, Y is 4,6-dimethoxy-1,3,5-triazin-2-yloxy.

In some embodiments, Y is N-(3-(dimethylamino)propyl)-N′-ethylcarbamimidoyloxy.

In some embodiments, the activating agent is selected from: thionyl chloride, 1H-benzo[d][1,2,3]triazol-1-ol, N1-((ethylimino)methylene)-N3,N3-dimethylpropane-1,3-diamine hydrochloride, and chlorodimethoxytriazine.

In some embodiments, the activating agent is thionyl chloride.

In some embodiments, the activating agent is 1H-benzo[d][1,2,3]triazol-1-ol.

In some embodiments, the activating agent is 1H-benzo[d][1,2,3]triazol-1-ol and said reacting of a compound of Formula IV with an activating agent is carried out in the further presence of N1-((ethylimino)methylene)-N3,N3-dimethylpropane-1,3-diamine hydrochloride.

In some embodiments, the activating agent is N1-((ethylimino)methylene)-N3,N3-dimethylpropane-1,3-diamine hydrochloride.

In some embodiments, the activating agent is chlorodimethoxytriazine.

In some embodiments, the molar ratio of said activating agent and said compound of Formula IV is about 10:1 to about 1:1.

In some embodiments, the molar ratio of said activating agent and said compound of Formula IV is about 7:1 to about 4:1.

In some embodiments, the molar ratio of said activating agent and said compound of Formula IV is about 2:1 to about 1:1.

In some embodiments, the molar ratio of said activating agent and said compound of Formula IV is about 1.2:1 to about 1:1.

In some embodiments, the molar ratio of said activating agent and said compound of Formula IV is about 1:1.

In some embodiments, the reacting of a compound of Formula IV with an activating agent is carried out in the presence of a base.

In some embodiments, the reacting of a compound of Formula IV with an activating agent is carried out in the presence of an organic base.

In some embodiments, the reacting of a compound of Formula IV with an activating agent is carried out in the presence of 4-methylmorpholine.

In some embodiments, the reacting of a compound of Formula IV with an activating agent is carried out in the substantial absence of solvent.

In some embodiments, the reacting of a compound of Formula IV with an activating agent is carried out in the presence of a polar solvent.

In some embodiments, the reacting of a compound of Formula IV with an activating agent is carried out in the presence of water.

In some embodiments, the reacting of a compound of Formula IV with an activating agent is carried out in the presence of an ether solvent.

In some embodiments, the reacting of a compound of Formula IV with an activating agent is carried out in the presence of tetrahydrofuran.

In some embodiments, the reacting of a compound of Formula IV with an activating agent is carried out in the presence of water and tetrahydrofuran.

In some embodiments, the reacting of a compound of Formula IV with an activating agent, is performed at a temperature of about −50° C. to about 100° C.

In some embodiments, the reacting of a compound of Formula IV with an activating agent, is performed at a temperature of about −25° C. to about 75° C.

In some embodiments, the reacting of a compound of Formula IV with an activating agent, is performed at a temperature of about 0° C. to about 50° C.

In some embodiments, the reacting of a compound of Formula IV with an activating agent, is performed at a temperature of about 10° C. to about 40° C.

In some embodiments, the reacting of a compound of Formula IV with an activating agent, is performed at a temperature of about 20° C. to about 30° C.

In some embodiments, the compound of Formula II has the following structure (IIa):

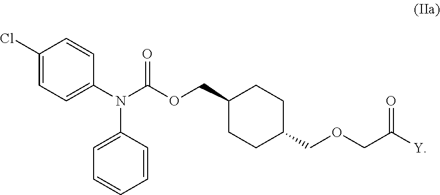

In some embodiments, the compound of Formula II has the following structure (IIb):

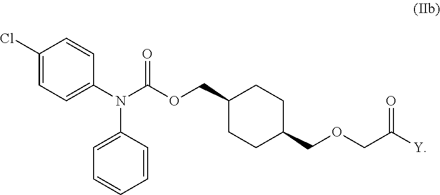

In some embodiments, the compound of Formula IV has the following structure (IVa):

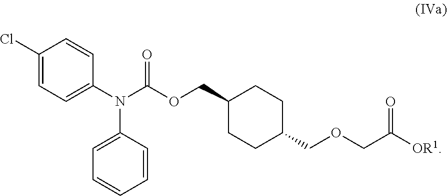

In some embodiments, the compound of Formula IV has the following structure (IVb):

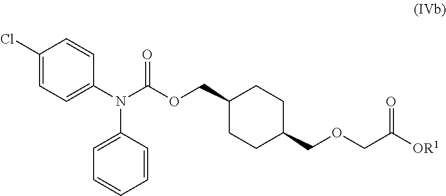

### Taurination Step

One aspect of the present invention pertains to processes for preparing a compound selected from: 2-(2-((4-(((4-chlorophenyl)(phenyl)carbamoyloxy)methyl)cyclohexyl)methoxy)acetamido)ethanesulfonic acid (Compound I), which has the following structure:

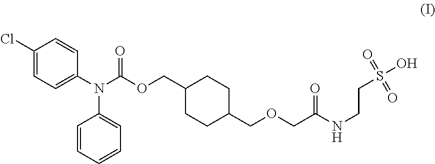

and pharmaceutically acceptable salts, solvates and hydrates thereof, comprising reacting a compound of Formula II:

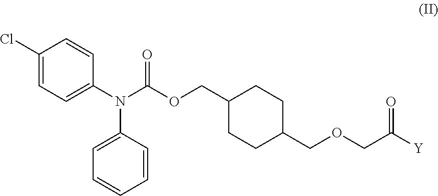

or a salt thereof; wherein: Y is an activating group; with taurine (Compound III):

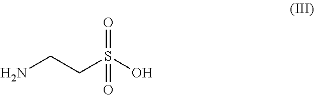

or a salt thereof; to form said Compound I or a pharmaceutically acceptable salt, solvate or hydrate thereof.

In some embodiments, Y is selected from: 1H-benzo[d][1,2,3]triazol-1-yloxy, 2,5-dioxo-3, sulfopyrrolidin-1-yloxy, 2,5-dioxopyrrolidin-1-yloxy, 3H-[1,2,3]triazolo[4,5-b]pyridin-3-yloxy, 4,6-dimethoxy-1,3,5-triazin-2-yloxy, 4-oxobenzo[d][1,2,3]triazin-3 (4H)-yloxy, 6-chloro-1H-benzo[d][1,2,3]triazol-1-yloxy, bis(2-oxooxazolidin-3-yl)phosphoryloxy, bis(dimethylamino)methoxy, bromo, chloro, diisopropylcarbamimidoyloxy, iodo, N-(3-(dimethylamino)propyl)-N′-ethylcarbamimidoyloxy, N,N′-dicyclohexylcarbamimidoyloxy, tris(dimethylamino)phosphoniooxy, and tris(pyrrolidino)phosphoniooxy.

One aspect of the present invention pertains to processes for preparing a compound selected from: 2-(2-((4-(((4-chlorophenyl)(phenyl)carbamoyloxy)methyl)cyclohexyl)methoxy)acetamido)ethanesulfonic acid (Compound I), which has the following structure:

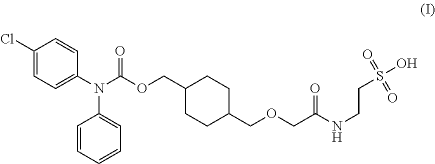

and pharmaceutically acceptable salts, solvates and hydrates thereof, comprising reacting a compound of Formula II:

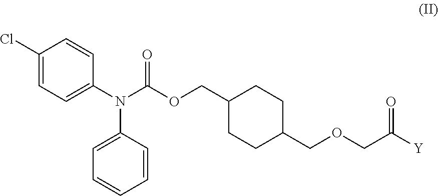

or a salt thereof; wherein: Y is an activating group; with taurine (Compound III):

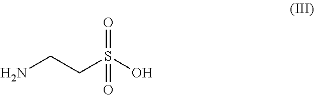

or a salt thereof; to form said Compound I or a pharmaceutically acceptable salt, solvate or hydrate thereof;

provided that:
(i) Y is other than Cl; and
(ii) Y is other than 1H-benzo[d][1,2,3]triazol-1-yloxy.

In some embodiments, Y is selected from: 4,6-dimethoxy-1,3,5-triazin-2-yloxy and N-(3-(dimethylamino)propyl)-N′-ethylcarbamimidoyloxy.

In some embodiments, Y is 4,6-dimethoxy-1,3,5-triazin-2-yloxy.

In some embodiments, Y is N-(3-(dimethylamino)propyl)-N′-ethylcarbamimidoyloxy.

In some embodiments, the compound of Formula II, or a salt thereof, is purified before the reacting of the compound of Formula II, or a salt thereof, with taurine, or a salt thereof.

In some embodiments, the compound of Formula II, or a salt thereof is substantially pure.

In some embodiments, the reacting of a compound of Formula II, or a salt thereof, with taurine, or a salt thereof, is performed in situ.

In some embodiments, the molar ratio of said compound of Formula II and said taurine, or a salt thereof, is about 10:1 to about 1:1.

In some embodiments, the molar ratio of said compound of Formula II and said taurine, or a salt thereof, is about 6:1 to about 2:1.

In some embodiments, the molar ratio of said compound of Formula II and said taurine, or a salt thereof, is about 4:1 to about 2:1.

In some embodiments, the molar ratio of said compound of Formula II and said taurine, or a salt thereof, is about 2:1 to about 1:1.

In some embodiments, the molar ratio of said compound of Formula II and said taurine, or a salt thereof, is about 1:1.

In some embodiments, the reacting a compound of Formula II, or a salt thereof, with taurine, or a salt thereof is carried out in the presence of a base.

In some embodiments, the reacting a compound of Formula II, or a salt thereof, with taurine, or a salt thereof is carried out in the presence of an organic base.

In some embodiments, the reacting a compound of Formula II, or a salt thereof, with taurine, or a salt thereof is carried out in the presence of 4-methylmorpholine.

In some embodiments, the reacting a compound of Formula II, or a salt thereof, with taurine, or a salt thereof is carried out in the presence of an inorganic base.

In some embodiments, the reacting a compound of Formula II, or a salt thereof, with taurine, or a salt thereof is carried out in the presence of sodium hydroxide.

In some embodiments, the reacting a compound of Formula II, or a salt thereof, with taurine, or a salt thereof is carried out in the presence of a polar solvent.

In some embodiments, the reacting a compound of Formula II, or a salt thereof, with taurine, or a salt thereof is carried out in the presence of water.

In some embodiments, the reacting a compound of Formula II, or a salt thereof, with taurine, or a salt thereof is carried out in the presence of an ether solvent.

In some embodiments, the reacting a compound of Formula II, or a salt thereof, with taurine, or a salt thereof is carried out in the presence of tetrahydrofuran.

In some embodiments, the reacting a compound of Formula II, or a salt thereof, with taurine, or a salt thereof is carried out in the presence of water and tetrahydrofuran.

In some embodiments, the reacting a compound of Formula II, or a salt thereof, with taurine, or a salt thereof, is performed at a temperature of about −50° C. to about 100° C.

In some embodiments, the reacting a compound of Formula II, or a salt thereof, with taurine, or a salt thereof, is performed at a temperature of about −25° C. to about 75° C.

In some embodiments, the reacting a compound of Formula II, or a salt thereof, with taurine, or a salt thereof, is performed at a temperature of about 0° C. to about 50° C.

In some embodiments, the reacting a compound of Formula II, or a salt thereof, with taurine, or a salt thereof, is performed at a temperature of about 10° C. to about 40° C.

In some embodiments, the reacting a compound of Formula II, or a salt thereof, with taurine, or a salt thereof, is performed at a temperature of about 20° C. to about 30° C.

In some embodiments, Compound I is 2-(2-(((1r,4r)-4-(((4-chlorophenyl)(phenyl)carbamoyloxy)methyl)cyclohexyl)methoxy)acetamido)ethanesulfonic acid (Compound Ia).

In some embodiments, Compound I is 2-(2-(((1s,4s)-4-(((4-chlorophenyl)(phenyl)carbamoyloxy)methyl)cyclohexyl)methoxy)acetamido)ethanesulfonic acid (Compound Ib).

### Salt Forming Step

One aspect of the present invention pertains to processes for preparing a salt of 2-(2-((4-(((4-chlorophenyl)(phenyl)carbamoyloxy)methyl)cyclohexyl)methoxy)acetamido)ethanesulfonic acid (Compound I):

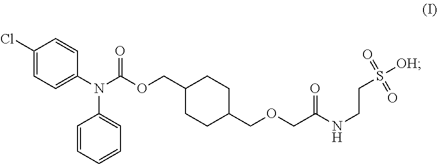

comprising reacting said Compound I with a salt-forming base to form said salt of Compound I;
wherein the anion of said salt of Compound I is 2-(2-((4-(((4-chlorophenyl)(phenyl)carbamoyloxy)methyl)cyclohexyl)methoxy)acetamido)ethanesulfonate;
provided that the cation of said salt of Compound I is other than sodium.

In some embodiments, the salt is selected from: a potassium salt, a calcium salt, a magnesium salt, a TRIS salt and an L-arginine salt.

In some embodiments, the salt is a potassium salt.

In some embodiments, the salt is a calcium salt.

In some embodiments, the salt is a magnesium salt.

In some embodiments, the salt is a TRIS salt.

In some embodiments, the salt is an L-arginine salt

In some embodiments, the reacting is carried out in the presence of a polar solvent.

In some embodiments, the reacting is carried out in the presence of water.

In some embodiments, the reacting is carried out in the presence of an ether solvent.

In some embodiments, the reacting is carried out in the presence of tetrahydrofuran.

In some embodiments, the reacting is carried out in the presence of water and tetrahydrofuran.

In some embodiments, the reacting is carried out at a temperature of about −10° C. to about reflux temperature.

In some embodiments, the reacting is carried out at a temperature of about 10° C. to about 80° C.

In some embodiments, the reacting is carried out at a temperature of about 20° C. to about 80° C.

In some embodiments, the salt-forming base is a metal hydroxide.

In some embodiments, the salt-forming base is a potassium hydroxide.

In some embodiments, the salt is a salt of 2-(2-(((1r,4r)-4-(((4-chlorophenyl)(phenyl)carbamoyloxy)methyl)cyclohexyl)methoxy)acetamido)ethanesulfonic acid (Compound Ia).

In some embodiments, the salt is a salt of 2-(2-(((1s,4s)-4-(((4-chlorophenyl)(phenyl)carbamoyloxy)methyl)cyclohexyl)methoxy)acetamido)ethanesulfonic acid (Compound Ib).

### Salts of the Present Invention

One aspect of the present invention pertains to salts of 2-(2-((4-(((4-chlorophenyl)(phenyl)carbamoyloxy)methyl)cyclohexyl)methoxy)acetamido)ethanesulfonic acid (Compound I):

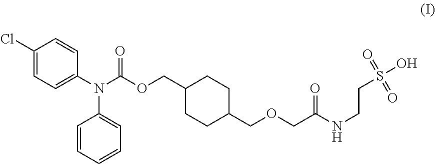

wherein the anion of the salt of Compound I is 2-(2-((4-(((4-chlorophenyl)(phenyl)carbamoyloxy)methyl)cyclohexyl)methoxy)acetamido)ethanesulfonate;
provided that the cation of the salt of Compound I is other than sodium.

In some embodiments, the cation is selected from: potassium, calcium, magnesium, TRIS, and L-arginine.

In some embodiments, the cation is potassium.

In some embodiments, the cation is calcium.

In some embodiments, the cation is magnesium.

In some embodiments, the cation is TRIS.

In some embodiments, the cation is L-arginine.

In some embodiments, the salt of Compound I has a purity of 80% or greater.

In some embodiments, the salt of Compound I has a purity of 90% or greater.

In some embodiments, the salt of Compound I has a purity of 95% or greater.

In some embodiments, the salt of Compound I has a purity of 99% or greater.

In some embodiments, the salt of Compound I has a purity of 99.5% or greater.

In some embodiments, the salt is a salt of 2-(2-(((1r,4r)-4-(((4-chlorophenyl)(phenyl)carbamoyloxy)methyl)cyclohexyl)methoxy)acetamido)ethanesulfonic acid (Compound Ia).

In some embodiments, the salt is a salt of 2-(2-(((1s,4s)-4-(((4-chlorophenyl)(phenyl)carbamoyloxy)methyl)cyclohexyl)methoxy)acetamido)ethanesulfonic acid (Compound Ib).

The salts of the present invention can also include all isotopes of atoms occurring in the intermediates and/or final compounds. Isotopes include those atoms having the same atomic number but different mass numbers. For example, isotopes of hydrogen include deuterium and tritium.

It is understood and appreciated that salts of Compound I may have one or more chiral centers and therefore can exist as enantiomers and/or diastereoisomers. The invention is understood to extend to and embrace all such enantiomers, diastereoisomers and mixtures thereof, including but not limited to racemates. It is understood that salts of Compound I and formulae used throughout this disclosure are intended to represent all individual enantiomers and mixtures thereof, unless stated or shown otherwise.

It is understood and appreciated that Compound I and salts thereof exist as meso isomers. Such meso isomers may be referred to as cis and trans. The cis meso isomers of Compound I and salts thereof are named herein using the prefix (1s,4s) and the trans meso isomers of Compound I and salts thereof are named herein using the prefix (1r,4r) as shown below:

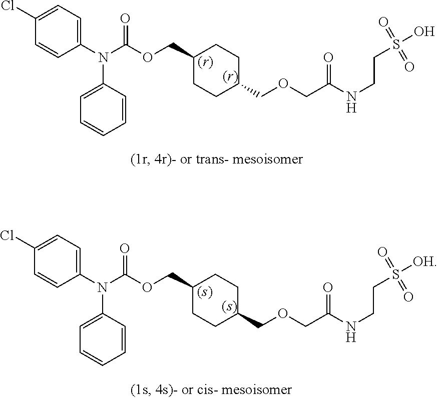

The invention is understood to extend to and embrace all such mesoisomers and mixtures thereof, including but not limited to a 1:1 mixture of mesoisomers. It is understood that salts, solvates, hydrates, and crystalline forms of the present invention; compounds prepared by the processes of the present invention, and pharmaceutically acceptable salts, solvates, hydrates, and crystalline forms thereof; and formulae used throughout this disclosure are intended to represent all individual mesoisomers and all mixtures thereof, unless stated or shown otherwise.

### Hydrates and Solvates

It is understood that when the phrase “pharmaceutically acceptable salts, solvates, and hydrates” or the phrase “pharmaceutically acceptable salt, solvate, or hydrate” is used when referring to compounds described herein, it embraces pharmaceutically acceptable solvates and/or hydrates of the compounds, pharmaceutically acceptable salts of the compounds, as well as pharmaceutically acceptable solvates and/or hydrates of pharmaceutically acceptable salts of the compounds. It is also understood that when the phrase “pharmaceutically acceptable solvates and hydrates” or the phrase “pharmaceutically acceptable solvate or hydrate” is used when referring to salts described herein, it embraces pharmaceutically acceptable solvates and/or hydrates of such salts.

It will be apparent to those skilled in the art that the dosage forms described herein may comprise, as the active component, either a compound described herein or a pharmaceutically acceptable salt or as a pharmaceutically acceptable solvate or hydrate thereof. Moreover, various hydrates and solvates of the compounds described herein and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present invention pertains to methods of administering hydrates and solvates of compounds described herein and/or their pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

One aspect of the present invention pertains to solvates and hydrates of 2-(2-((4-(((4-chlorophenyl)(phenyl)carbamoyloxy)methyl)cyclohexyl)methoxy)acetamido)ethanesulfonic acid (Compound I), and pharmaceutically acceptable salts thereof. In some embodiments, the solvate or hydrate is a solvate or hydrate of 2-(2-(((1r,4r)-4-(((4-chlorophenyl)(phenyl)carbamoyloxy)methyl)cyclohexyl)methoxy)acetamido)ethanesulfonic acid (Compound Ia) or a pharmaceutically acceptable salt thereof.

The embodiments of the present invention include every combination of one or more solvate or hydrate of a salt selected from the following group:
potassium 2-(2-(((1r,4r)-4-(((4-chlorophenyl)(phenyl)carbamoyloxy)methyl)cyclohexyl)methoxy)acetamido)ethanesulfonate hydrate;
magnesium 2-(2-(((1r,4r)-4-(((4-chlorophenyl)(phenyl)carbamoyloxy)methyl)cyclohexyl)methoxy)acetamido)ethanesulfonate hydrate;
calcium 2-(2-(((1r,4r)-4-(((4-chlorophenyl)(phenyl)carbamoyloxy)methyl)cyclohexyl)methoxy)acetamido)ethanesulfonate hydrate I;
calcium 2-(2-(((1r,4r)-4-(((4-chlorophenyl)(phenyl)carbamoyloxy)methyl)cyclohexyl)methoxy)acetamido)ethanesulfonate hydrate II;
calcium 2-(2-(((1r,4r)-4-(((4-chlorophenyl)(phenyl)carbamoyloxy)methyl)cyclohexyl)methoxy)acetamido)ethanesulfonate hydrate III;
calcium 2-(2-(((1r,4r)-4-(((4-chlorophenyl)(phenyl)carbamoyloxy)methyl)cyclohexyl)methoxy)acetamido)ethanesulfonate hydrate IV;
TRIS 2-(2-((1r,4r)-4-(((4-chlorophenyl)(phenyl)carbamoyloxy)methyl)cyclohexyl)methoxy)acetamido)ethanesulfonate hydrate; and

L-arginine 2-(2-(((1r,4r)-4-(((4-chlorophenyl)(phenyl)carbamoyloxy)methyl)cyclohexyl)methoxy)acetamido)ethanesulfonate hydrate.

### Crystalline Forms

A further aspect of the present invention pertains to crystalline forms of 2-(2-((4-(((4-chlorophenyl)(phenyl)carbamoyloxy)methyl)cyclohexyl)methoxy)acetamido)ethanesulfonic acid (Compound I), and pharmaceutically acceptable salts, solvates and hydrates thereof. In some embodiments, the crystalline form is a crystalline form of 2-(2-(((1r,4r)-4-(((4-chlorophenyl)(phenyl)carbamoyloxy)methyl)cyclohexyl)methoxy)acetamido)ethanesulfonic acid (Compound Ia) or a pharmaceutically acceptable salt, solvate, or hydrate thereof.

A further aspect of the present invention pertains to crystalline forms of a compound selected from: a pharmaceutically acceptable salt of 2-(2-((4-(((4-chlorophenyl)(phenyl)carbamoyloxy)methyl)cyclohexyl)methoxy)acetamido)ethanesulfonic acid (Compound I), and solvates and hydrates thereof. In some embodiments, the crystalline form is a crystalline form of a compound selected from: a pharmaceutically acceptable salt of 2-(2-(((1r,4r)-4-(((4-chlorophenyl)(phenyl)carbamoyloxy)methyl)cyclohexyl)methoxy)acetamido)ethanesulfonic acid (Compound Ia), and solvates and hydrates thereof.

A further aspect of the present invention pertains to crystalline forms of a compound selected from: a pharmaceutically acceptable salt of 2-(2-((4-(((4-chlorophenyl)(phenyl)carbamoyloxy)methyl)cyclohexyl)methoxy)acetamido)ethanesulfonic acid (Compound I), and solvates and hydrates thereof; provided that said compound is other than sodium 2-(2-((4-(((4-chlorophenyl)(phenyl)carbamoyloxy)methyl)cyclohexyl)methoxy)acetamido)ethanesulfonate; and further provided that said compound is other than sodium 2-(2-((4-(((4-chlorophenyl)(phenyl)carbamoyloxy)methyl)cyclohexyl)methoxy)acetamido)ethanesulfonate hydrate. In some embodiments, the crystalline form is a crystalline form of a compound selected from: a pharmaceutically acceptable salt of 2-(2-(((1r,4r)-4-(((4-chlorophenyl)(phenyl)carbamoyloxy)methyl)cyclohexyl)methoxy)acetamido)ethanesulfonic acid (Compound Ia), and solvates and hydrates thereof; provided that said compound is other than sodium 2-(2-(((1r,4r)-4-(((4-chlorophenyl)(phenyl)carbamoyloxy)methyl)cyclohexyl)methoxy)acetamido)ethanesulfonate;

and further provided that said compound is other than sodium 2-(2-(((1r,4r)-4-(((4-chlorophenyl)(phenyl)carbamoyloxy)methyl)cyclohexyl)methoxy)acetamido)ethanesulfonate hydrate.

In some embodiments, the crystalline form is a crystalline form of 2-(2-(((1r,4r)-4-(((4-chlorophenyl)(phenyl)carbamoyloxy)methyl)cyclohexyl)methoxy)acetamido)ethanesulfonic acid hydrate

Crystalline forms can be identified by their unique solid state signature with respect to, for example, differential scanning calorimetry (DSC), X-ray powder diffraction (PXRD), and other solid state methods.

Further characterization with respect to water or solvent content of crystalline forms can be gauged by any of the following methods for example, thermogravimetric analysis (TGA), DSC and the like.

For DSC, it is known that the temperatures observed will depend upon sample purity, the rate of temperature change, as well as sample preparation technique and the particular instrument employed. Thus, the values reported herein relating to DSC thermograms can vary by plus or minus about 4° C. The values reported herein relating to DSC thermograms can also vary by plus or minus about 20 joules per gram.

In some embodiments, the DSC thermogram values reported herein relate to dehydration events. When DSC thermogram values reported herein relate to dehydration events, the values reported herein are estimates. Scan rate and pan closure can influence DSC values for dehydration events, which can vary by plus or minus about 25° C. DSC values for dehydration events reported herein were recorded using a sample in an aluminum pan with an uncrimped lid and a scan rate of 10° C./min.

For PXRD, the relative intensities of the peaks can vary, depending upon the sample preparation technique, the sample mounting procedure and the particular instrument employed. Moreover, instrument variation and other factors can often affect the 2θ values. Therefore, the peak assignments of diffraction patterns can vary by plus or minus about 0.2°2θ.

For TGA, the features reported herein can vary by plus or minus about 5° C. The TGA features reported herein can also vary by plus or minus about 2% weight change due to, for example, sample variation.

Further characterization with respect to hygroscopicity of the crystalline forms can be gauged by, for example, dynamic moisture sorption (DMS). The DMS features reported herein can vary by plus or minus about 5% relative humidity. The DMS features reported herein can also vary by plus or minus about 5% weight change.

### 1. Compound Ia Free Acid Hydrate.

One aspect of the present invention is directed to a crystalline form of Compound Ia free acid hydrate. The physical properties of the crystalline form of Compound Ia free acid hydrate are summarized in Table 1 below.

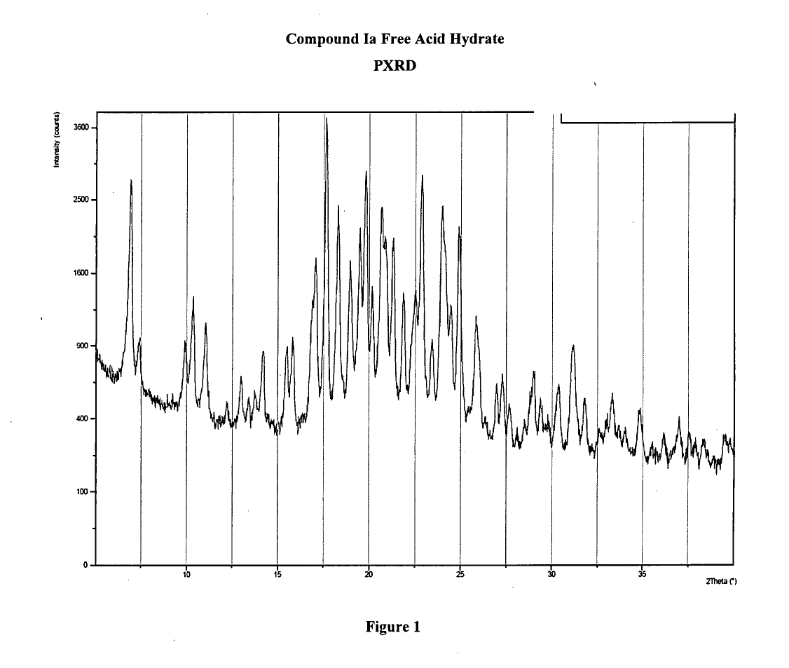

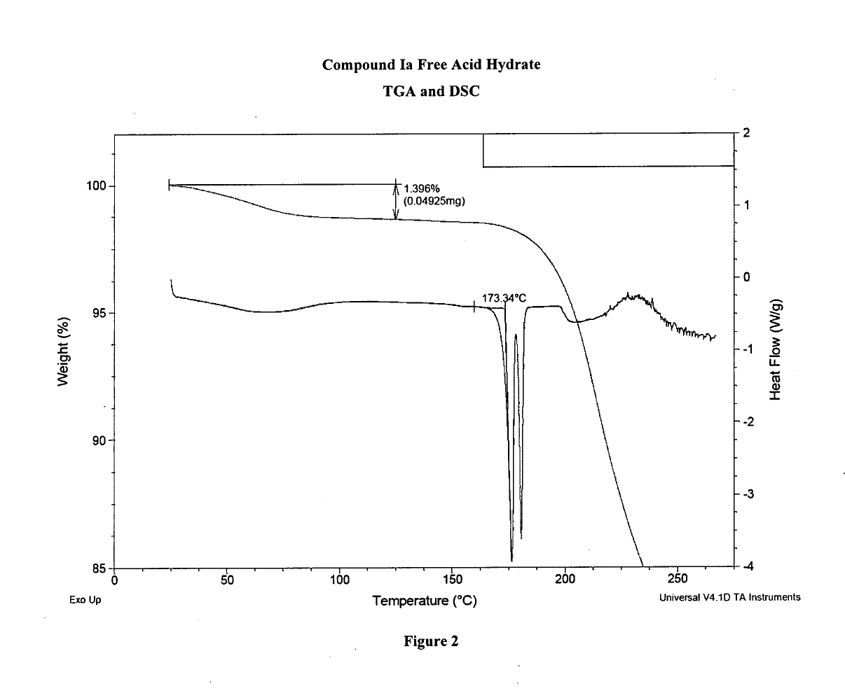

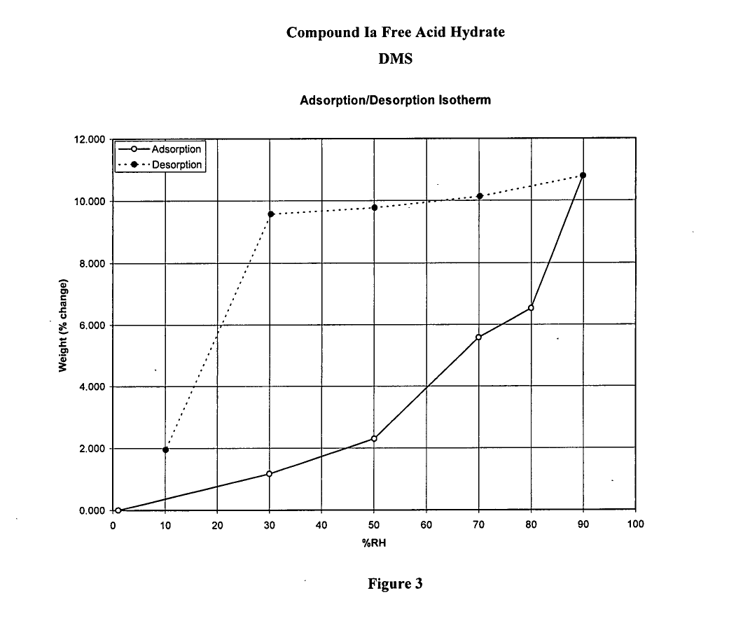

TABLE 1

| | Compound Ia Free Acid Hydrate |
|---|---|
| PXRD | FIG. 1: Peaks of ≧50% relative intensity at 6.9, 17.6, 18.3, |
| | 19.5, 19.8, 20.7, 22.9, 24.0, and 24.9 °2θ |
| TGA | FIG. 2: Decrease in weight of about 1.4% out to about 125° C. |
| DSC | FIG. 2: First endotherm extrapolated onset temperature: |
| | about 173° C. |
| DMS | FIG. 3: Increase of about 10% weight at about 90% relative |
| | humidity |

Certain X-ray powder diffraction peaks for the crystalline form of Compound Ia free acid hydrate are shown in Table 2 below.

TABLE 2

| Pos. [°2θ] | Rel. Int. [%] |
|---|---|
| 5.1308 | 2.28 |
| 6.9090 | 66.63 |
| 7.3709 | 11.76 |
| 9.8929 | 15.25 |
| 10.3336 | 27.85 |
| 11.0216 | 21.79 |
| 12.2022 | 4.18 |
| 12.9913 | 8.62 |
| 13.4027 | 5.14 |
| 13.7527 | 6.34 |
| 14.1856 | 15.78 |
| 15.4876 | 17.02 |
| 15.8109 | 18.97 |
| 16.8343 | 25.36 |
| 17.0463 | 41.53 |
| 17.6410 | 100.00 |
| 18.2927 | 62.01 |
| 18.9495 | 40.65 |
| 19.4767 | 52.36 |
| 19.7948 | 75.88 |
| 20.1479 | 31.76 |
| 20.6709 | 59.68 |
| 20.8678 | 47.19 |
| 21.2987 | 46.82 |
| 21.8671 | 29.58 |
| 22.5164 | 29.55 |
| 22.8697 | 73.83 |
| 23.4336 | 17.56 |
| 23.9764 | 61.77 |
| 24.1735 | 41.44 |
| 24.4624 | 27.07 |
| 24.9012 | 53.74 |
| 25.8391 | 25.53 |
| 26.0269 | 15.05 |
| 26.3493 | 3.25 |
| 26.9662 | 9.78 |
| 27.2763 | 12.24 |
| 27.6750 | 6.53 |
| 28.5044 | 4.09 |
| 28.0921 | 2.35 |
| 29.0112 | 13.00 |
| 29.3855 | 7.66 |
| 30.3553 | 10.59 |
| 31.1063 | 19.37 |
| 31.8057 | 8.63 |
| 32.6318 | 3.75 |
| 33.0110 | 4.93 |
| 33.2946 | 9.95 |
| 33.6806 | 4.77 |
| 34.0112 | 4.02 |
| 34.8167 | 7.65 |
| 35.4815 | 2.32 |
| 36.1415 | 3.82 |
| 36.9786 | 6.03 |
| 37.5367 | 4.12 |
| 37.8591 | 3.02 |
| 38.3185 | 3.22 |
| 39.4671 | 3.57 |

The crystalline form of Compound Ia free acid hydrate, was partially hydrated at the time of analyses. TGA showed about 1.4% weight loss (0.42 mol) out to 125° C. DSC showed the crystalline form of Compound Ia free acid hydrate had two closely spaced endotherms with an onset of 173° C. for the first.

The crystalline form of Compound Ia free acid hydrate was hygroscopic by DMS analysis, picking up just over 10% weight at 90% RH. It appears to form a new hydrate crystal phase at high % RH values. This new hydrate form then quickly loses the higher level of water associated with it when the humidity is lowered to between 30 and 10% RH.

One aspect of the present invention is directed to a crystalline form of Compound Ia free acid hydrate having an X-ray powder diffraction pattern comprising a peak, in terms of 2θ, at about 17.6. In some embodiments, the crystalline form has an X-ray powder diffraction pattern comprising a peak, in terms of 2θ, at about 19.8°. In some embodiments, the crystalline form has an X-ray powder diffraction pattern comprising a peak, in terms of 2θ, at about 17.6° and about 22.9°. In some embodiments, the crystalline form has an X-ray powder diffraction pattern comprising a peak, in terms of 2θ, at about 17.6° and about 19.8°. In some embodiments, the crystalline form has an X-ray powder diffraction pattern comprising a peak, in terms of 2θ, at about 17.6°, about 19.8°, and about 22.9°. In some embodiments, the crystalline form has an X-ray powder diffraction pattern comprising a peak, in terms of 2θ, at about 17.6°, about 19.8°, about 22.9°, about 6.9°, about 18.3°, about 24.0°, about 20.7° and about 19.5°. In yet further embodiments, the crystalline form has an X-ray powder diffraction pattern substantially as shown in FIG. 1, wherein by “substantially” is meant that the reported peaks can vary by about ±0.2°2θ.

In some embodiments, the crystalline form of Compound Ia free acid hydrate has a differential scanning calorimetry thermogram comprising an endotherm with an extrapolated onset temperature between about 165° C. and about 180° C. In some embodiments, the crystalline form has a differential scanning calorimetry thermogram comprising an endotherm with an extrapolated onset temperature at about 173° C. In further embodiments, the crystalline form has a differential scanning calorimetry thermogram substantially as shown in FIG. 2, wherein by “substantially” is meant that the reported DSC features can vary by about ±4° C. and that the reported DSC features can vary by about ±20 joules per gram.

In some embodiments, the crystalline form of Compound Ia free acid hydrate has a thermogravimetric analysis profile substantially as shown in FIG. 2, wherein by “substantially” is meant that the reported TGA features can vary by about ±5° C., and that that the reported TGA features can vary by about ±2% weight change.

In some embodiments, the crystalline form of Compound Ia free acid hydrate has a dynamic moisture sorption (DMS) profile substantially as shown in FIG. 3, wherein by “substantially” is meant that the reported DMS features can vary by about ±5% relative humidity, and that the DMS features reported herein can vary by about ±5% weight change.

### 2. Compound Ia Sodium Salt Hydrate I.

One aspect of the present invention is directed to a crystalline form of Compound Ia sodium salt hydrate I. The physical properties of the crystalline form of Compound Ia sodium salt hydrate I are summarized in Table 3 below.

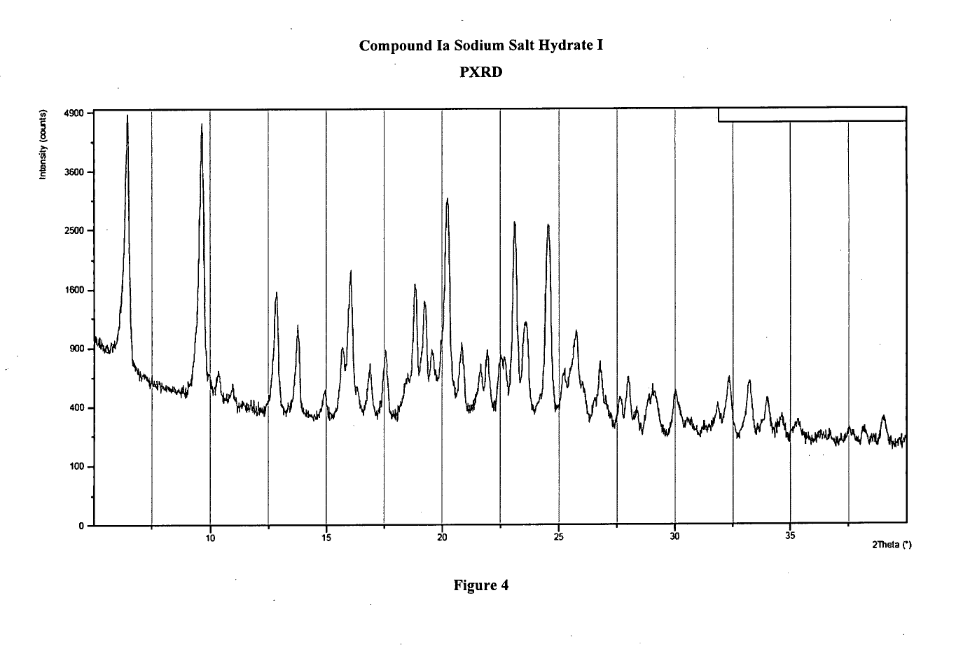

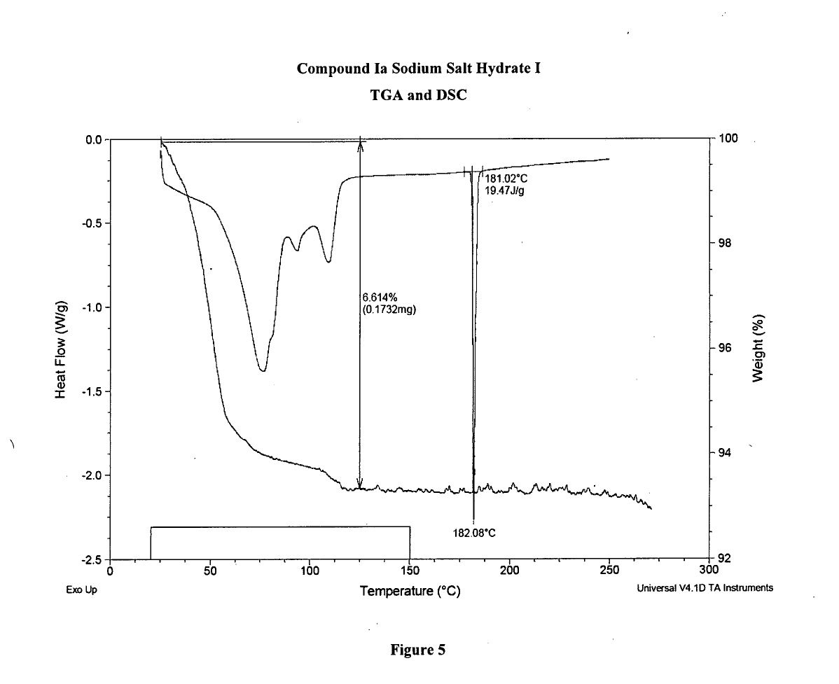

TABLE 3

| | Compound Ia Sodium Salt Hydrate I |
|---|---|
| PXRD | FIG. 4: Peaks of ≧30% relative intensity at 6.4, 9.6, 20.2, |
| | 23.1, 24.6, 16.0, and 18.8 °2θ |
| TGA | FIG. 5: Decrease in weight of about 6.6% out to about 125° C. |
| DSC | FIG. 5: Post-dehydration endotherm extrapolated onset |
| | temperature: about 181° C.; |

Certain X-ray powder diffraction peaks for the crystalline form of Compound Ia sodium salt hydrate I are shown in Table 4 below.

TABLE 4

| Pos. [°2θ] | Rel. Int. [%] |
|---|---|
| 6.4416 | 100.00 |
| 9.6376 | 99.31 |
| 10.3551 | 6.63 |
| 10.9488 | 4.19 |
| 12.8407 | 29.06 |
| 13.7690 | 19.82 |
| 14.9417 | 4.62 |
| 15.6935 | 14.32 |
| 16.0497 | 36.37 |
| 16.8844 | 10.78 |
| 17.5669 | 13.84 |
| 18.5285 | 8.04 |
| 18.8381 | 33.10 |
| 19.2536 | 26.47 |
| 19.5789 | 13.28 |
| 20.2205 | 65.22 |
| 20.8414 | 15.42 |
| 21.6469 | 10.15 |
| 21.9445 | 13.23 |
| 22.5271 | 11.67 |
| 22.6700 | 11.26 |
| 23.1256 | 55.24 |
| 23.6488 | 20.26 |
| 24.5587 | 54.67 |
| 25.2281 | 9.27 |
| 25.7619 | 18.49 |
| 26.0779 | 6.40 |
| 26.7821 | 11.83 |
| 27.6617 | 5.39 |
| 28.0051 | 9.61 |
| 28.3839 | 3.33 |
| 28.8366 | 4.99 |
| 29.0825 | 6.82 |
| 30.0503 | 7.13 |
| 30.6273 | 2.45 |
| 31.8611 | 5.08 |
| 32.3451 | 10.18 |
| 33.2330 | 9.34 |
| 33.9905 | 6.53 |
| 34.6160 | 3.48 |
| 35.3372 | 2.69 |
| 36.4049 | 0.98 |
| 37.5587 | 1.74 |
| 38.1389 | 2.10 |
| 38.9835 | 3.75 |

TGA showed about 6.6% weight loss out to about 125° C. DSC showed a sharp post-dehydration endotherm with an extrapolated onset temperature of about 181° C.

One aspect of the present invention is directed to a crystalline form of Compound Ia sodium salt hydrate I having an X-ray powder diffraction pattern comprising a peak, in terms of 2θ, at about 6.4°. In some embodiments, the crystalline form has an X-ray powder diffraction pattern comprising a peak, in terms of 2θ, at about 9.6°. In some embodiments, the crystalline form has an X-ray powder diffraction pattern comprising a peak, in terms of 2θ, at about 6.4° and about 20.2°. In some embodiments, the crystalline form has an X-ray powder diffraction pattern comprising a peak, in terms of 2θ, at about 6.4° and about 9.6°. In some embodiments, the crystalline form has an X-ray powder diffraction pattern comprising a peak, in terms of 2θ, at about 6.4°, about 9.6°, and about 20.2°. In some embodiments, the crystalline form has an X-ray powder diffraction pattern comprising a peak, in terms of 2θ, at about 6.4°, about 9.6°, about 20.2°, about 23.1°, about 24.6°, about 16.0°, and about 18.8°. In yet further embodiments, the crystalline form has an X-ray powder diffraction pattern substantially as shown in FIG. 4, wherein by “substantially” is meant that the reported peaks can vary by about ±0.2°2θ.

In some embodiments, the crystalline form of Compound Ia sodium salt hydrate I has a differential scanning calorimetry thermogram comprising an endotherm with an extrapolated onset temperature between about 175° C. and about 190° C. In some embodiments, the crystalline form has a differential scanning calorimetry thermogram comprising an endotherm with an extrapolated onset temperature at about 181° C. In further embodiments, the crystalline form has a differential scanning calorimetry thermogram substantially as shown in FIG. 5, wherein by “substantially” is meant that the reported DSC features can vary by about ±4° C. and that the reported DSC features can vary by about ±20 joules per gram.

In some embodiments, the crystalline form of Compound Ia sodium salt hydrate I has a thermogravimetric analysis profile substantially as shown in FIG. 5, wherein by “substantially” is meant that the reported TGA features can vary by about ±5° C., and that that the reported TGA features can vary by about ±2% weight change.

### 3. Compound Ia Sodium Salt Hydrate II.

One aspect of the present invention is directed to a crystalline form of Compound Ia sodium salt hydrate II. The physical properties of the crystalline form of Compound Ia sodium salt hydrate II are summarized in Table 5 below.

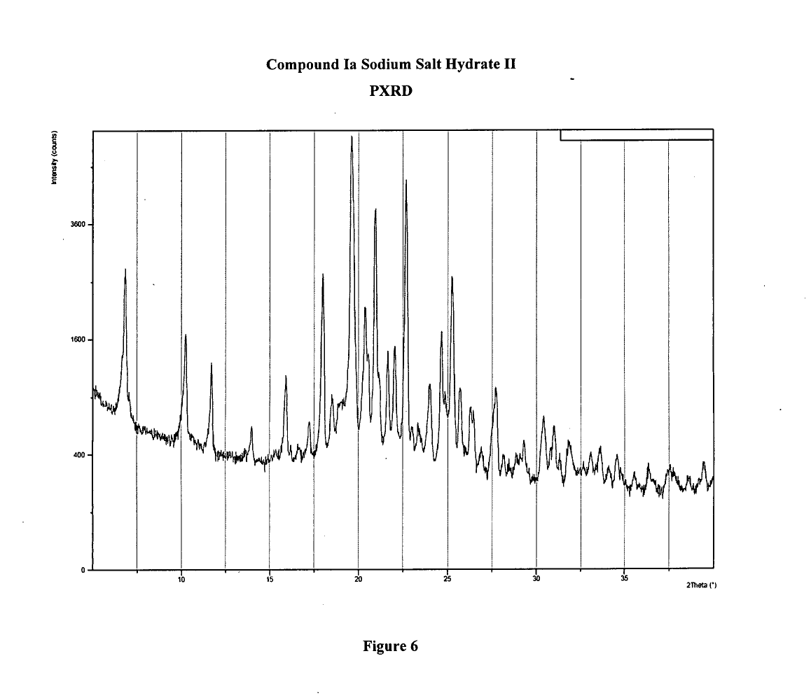

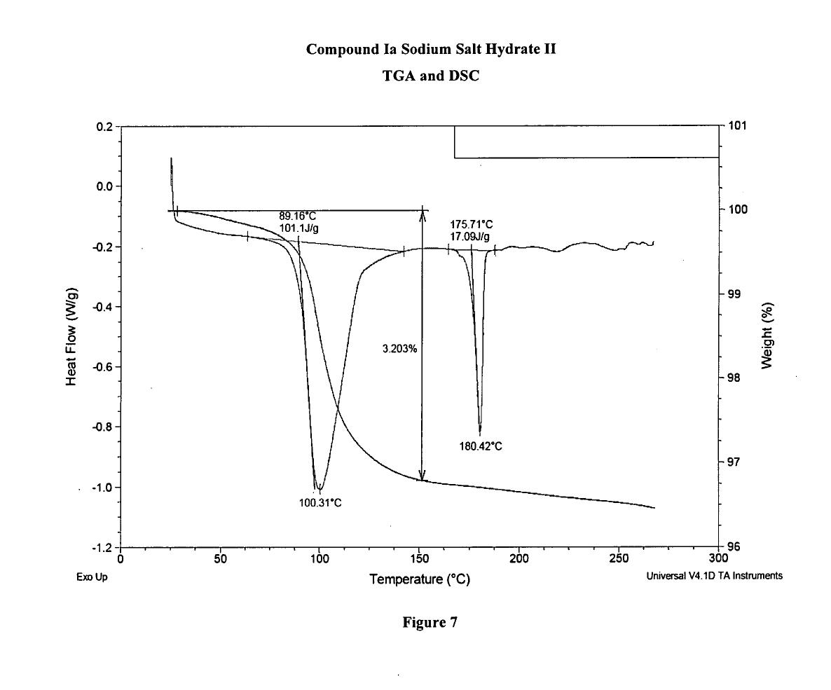

TABLE 5

| | Compound Ia Sodium Salt Hydrate II |
|---|---|
| PXRD | FIG. 6: Peaks of ≧30% relative intensity at 19.6, 22.7, 20.9, |
| | 25.3, 18.0, 6.8, and 20.4 °2θ |
| TGA | FIG. 7: Decrease in weight of about 3.2% out to about 150° C. |
| DSC | FIG. 7: Post-dehydration endotherm extrapolated onset |
| | temperature: about 176° C.; |

Certain X-ray powder diffraction peaks for the crystalline form of Compound Ia sodium salt hydrate II are shown in Table 6 below.

TABLE 6

| Pos. [°2θ] | Rel. Int. [%] |
|---|---|
| 6.8249 | 37.05 |
| 7.0496 | 5.49 |
| 10.2348 | 23.18 |
| 11.6975 | 16.8 |
| 13.9692 | 5.32 |
| 15.9002 | 15.43 |
| 16.6219 | 2.45 |
| 17.2274 | 5.93 |
| 17.9807 | 42.94 |
| 18.51 | 9.97 |
| 18.8688 | 9.02 |
| 19.5931 | 100 |
| 20.3635 | 32.1 |
| 20.5339 | 19.38 |
| 20.9382 | 66.06 |
| 21.6442 | 20.2 |
| 22.0346 | 21.72 |
| 22.666 | 79.7 |
| 23.0188 | 5.07 |
| 23.3695 | 4.83 |
| 23.9991 | 13.52 |
| 24.6558 | 26.52 |
| 25.2567 | 43.17 |
| 25.7071 | 13.35 |
| 26.2873 | 9.17 |
| 26.4858 | 7.83 |
| 26.9061 | 3.29 |
| 27.7173 | 13.75 |
| 28.1453 | 2.9 |
| 28.422 | 1.72 |
| 28.8842 | 2.97 |
| 29.3058 | 5.08 |
| 30.4223 | 9.46 |
| 30.9929 | 7.5 |
| 31.3261 | 3.12 |
| 31.86 | 5.34 |
| 33.0686 | 4.26 |
| 33.5975 | 4.61 |
| 34.0799 | 2.52 |
| 34.5579 | 4.35 |
| 35.5305 | 2.2 |
| 36.3393 | 2.75 |
| 37.492 | 2.64 |
| 38.5769 | 1.62 |
| 39.447 | 3.46 |

TGA showed about 3.2% weight loss out to 150° C. DSC showed an endotherm with an extrapolated onset temperature of about 176° C.

One aspect of the present invention is directed to a crystalline form of Compound Ia sodium salt hydrate II having an X-ray powder diffraction pattern comprising a peak, in terms of 2θ, at about 19.6°. In some embodiments, the crystalline form has an X-ray powder diffraction pattern comprising a peak, in terms of 2θ, at about 22.7°. In some embodiments, the crystalline form has an X-ray powder diffraction pattern comprising a peak, in terms of 2θ, at about 19.6° and about 20.9°. In some embodiments, the crystalline form has an X-ray powder diffraction pattern comprising a peak, in terms of 2θ, at about 19.6° and about 22.7°. In some embodiments, the crystalline form has an X-ray powder diffraction pattern comprising a peak, in terms of 2θ, at about 19.6°, about 22.7°, and about 20.9°. In some embodiments, the crystalline form has an X-ray powder diffraction pattern comprising a peak, in terms of 2θ, at about 19.6°, about 22.7°, about 20.9°, about 25.2°, about 18.0°, about 6.8°, and about 20.4°. In yet further embodiments, the crystalline form has an X-ray powder diffraction pattern substantially as shown in FIG. 6, wherein by “substantially” is meant that the reported peaks can vary by about ±0.2°2θ.

In some embodiments, the crystalline form of Compound Ia sodium salt hydrate II has a differential scanning calorimetry thermogram comprising an endotherm with an extrapolated onset temperature between about 170° C. and about 182° C. In some embodiments, the crystalline form has a differential scanning calorimetry thermogram comprising an endotherm with an extrapolated onset temperature at about 176° C. In further embodiments, the crystalline form has a differential scanning calorimetry thermogram substantially as shown in FIG. 7, wherein by “substantially” is meant that the reported DSC features can vary by about ±4° C. and that the reported DSC features can vary by about ±20 joules per gram.

In some embodiments, the crystalline form of Compound Ia sodium salt hydrate II has a thermogravimetric analysis profile substantially as shown in FIG. 7, wherein by “substantially” is meant that the reported TGA features can Vary by about ±5° C., and that that the reported TGA features can vary by about ±2% weight change.

### 4. Compound Ia Potassium Salt Hydrate.

One aspect of the present invention is directed to a crystalline form of Compound Ia potassium salt hydrate. The physical properties of the crystalline form of Compound Ia potassium salt hydrate are summarized in Table 7 below.

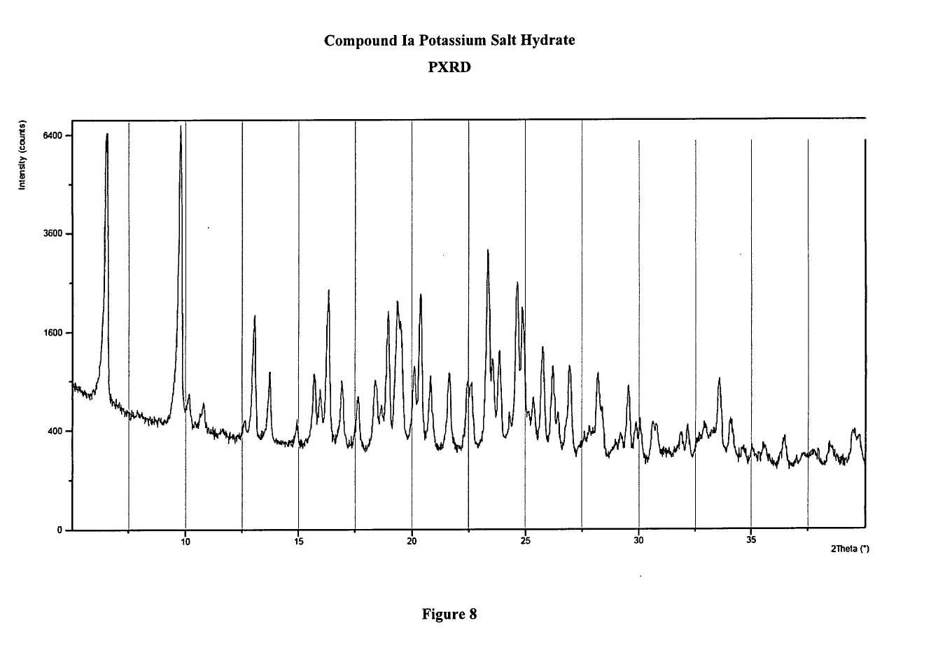

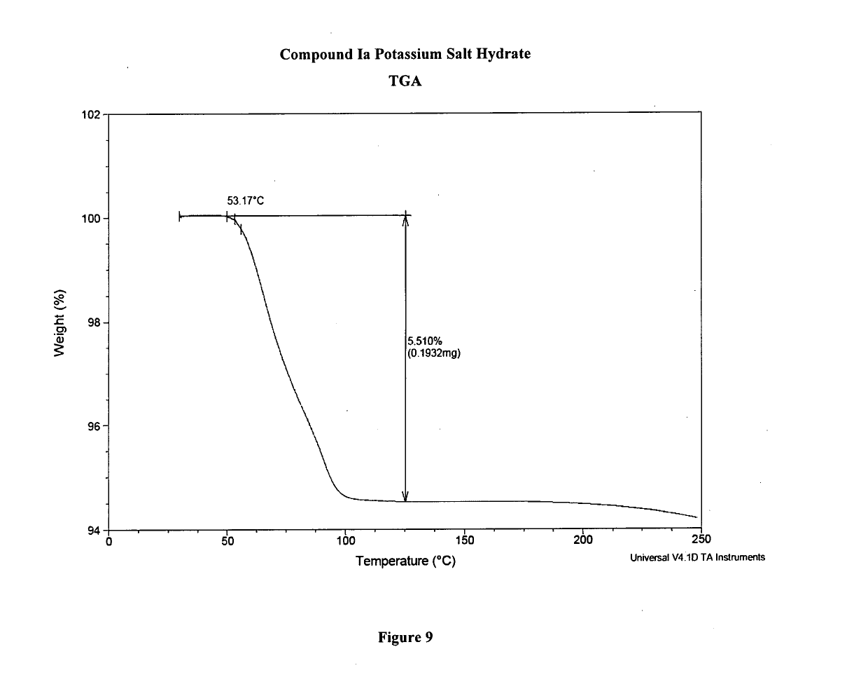

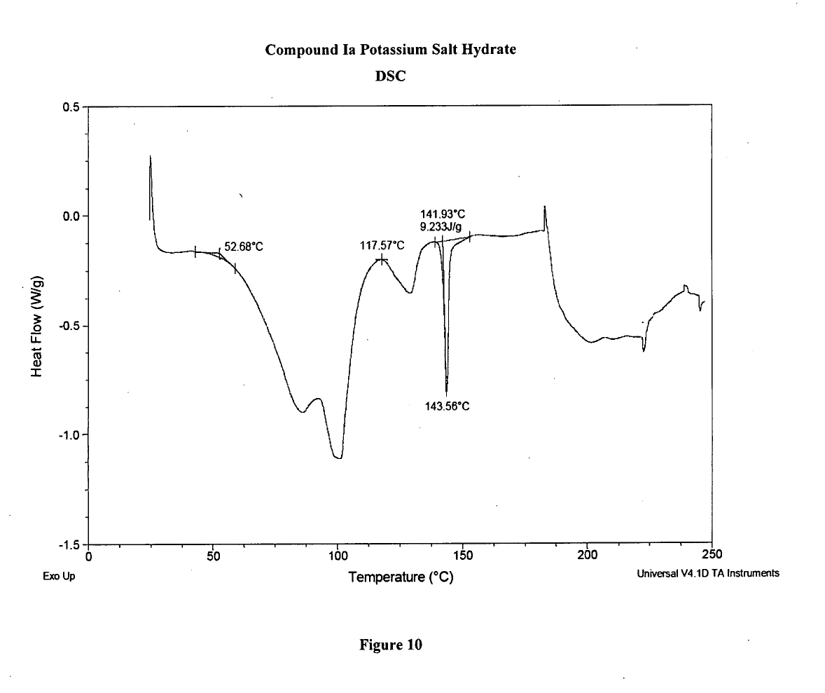

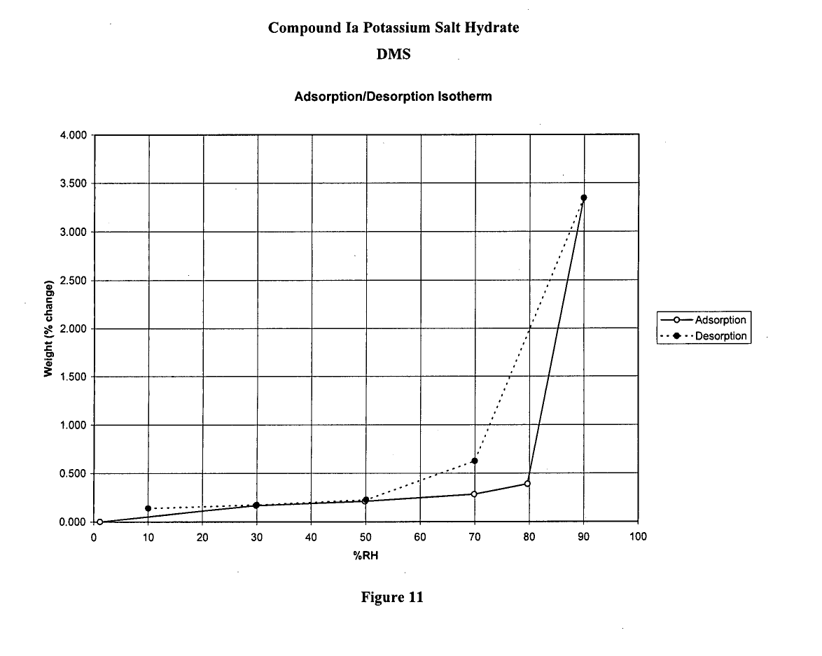

TABLE 7

| | Compound Ia Potassium Salt Hydrate |
|---|---|
| PXRD | FIG. 8: Peaks of ≧30% relative intensity at 9.8, 6.5, 23.4, 24.6, |
| | 16.3, 20.4, and 19.4 °2θ |
| TGA | FIG. 9: Decrease in weight of about 5.5% out to about 125° C. |
| DSC | FIG. 10: Small, sharp post-dehydration endotherm extrapolated |
| | onset temperature: about 142° C.; |
| DMS | FIG. 11: Increase of about 3-4% weight at about 90% relative |
| | humidity |

Certain X-ray powder diffraction peaks for the crystalline form of Compound Ia potassium salt hydrate are shown in Table 8 below.

TABLE 8

| Pos. [°2θ] | Rel. Int. [%] |
|---|---|
| 6.5325 | 91.03 |
| 9.7854 | 100 |
| 10.1638 | 5.42 |
| 10.7943 | 4 |
| 11.6241 | 1.18 |
| 12.6035 | 2.74 |
| 13.0475 | 25.01 |
| 13.726 | 11.49 |
| 14.927 | 3.31 |
| 15.7029 | 11.3 |
| 15.9568 | 8.18 |
| 16.3172 | 32.93 |
| 16.9231 | 9.95 |
| 17.6319 | 7.21 |
| 18.4041 | 10.38 |
| 18.6598 | 5.86 |
| 18.9594 | 27.11 |
| 19.3703 | 30.18 |
| 19.5093 | 23.45 |
| 20.1125 | 13.53 |
| 20.3885 | 32 |
| 20.8287 | 11.38 |
| 21.6514 | 11.9 |
| 22.4593 | 10.49 |
| 22.6193 | 9.85 |
| 23.3534 | 46.28 |
| 23.5662 | 15.2 |
| 23.8589 | 17.29 |
| 24.3019 | 4.89 |
| 24.6405 | 36.33 |
| 24.8809 | 27.83 |
| 25.3466 | 7.76 |
| 25.7712 | 18.46 |
| 26.2156 | 13.99 |
| 26.4388 | 5.38 |
| 26.9541 | 14.13 |
| 28.2014 | 12.66 |
| 28.3685 | 6.54 |
| 29.2138 | 2.73 |
| 29.5488 | 10.08 |
| 29.8911 | 4.04 |
| 30.0705 | 4.32 |
| 30.6398 | 4.58 |
| 30.7987 | 4.2 |
| 31.8875 | 3.09 |
| 32.171 | 3.81 |
| 32.9359 | 4.24 |
| 33.5665 | 11.92 |
| 34.0565 | 4.81 |
| 34.6107 | 1.61 |
| 35.0341 | 1.67 |
| 35.5499 | 1.92 |
| 36.4447 | 3.18 |
| 37.7373 | 1.47 |
| 38.4574 | 1.92 |
| 39.4792 | 3.57 |
| 39.7454 | 2.9 |

TGA showed about 5.5% weight loss out to 125° C., which is close to dihydrate stoichiometry. DSC showed small, sharp post-dehydration endotherm with an extrapolated onset temperature of about 142° C. This endotherm may be engulfed by more broad endotherms associated with dehydration. The crystalline form of Compound Ia potassium salt hydrate is slightly hygroscopic up to 90% RH, picking up 3-4% by weight.

One aspect of the present invention is directed to a crystalline form of Compound Ia potassium salt hydrate having an X-ray powder diffraction pattern comprising a peak, in terms of 2θ, at about 9.8°. In some embodiments, the crystalline form has an X-ray powder diffraction pattern comprising a peak, in terms of 2θ, at about 6.5°. In some embodiments, the crystalline form has an X-ray powder diffraction pattern comprising a peak, in terms of 2θ, at about 9.8° and about 23.4°. In some embodiments, the crystalline form has an X-ray powder diffraction pattern comprising a peak, in terms of 2θ, at about 9.8° and about 6.5°. In some embodiments, the crystalline form has an X-ray powder diffraction pattern comprising a peak, in terms of 2θ, at about 9.8°, about 6.5°, and about 23.4°. In some embodiments, the crystalline form has an X-ray powder diffraction pattern comprising a peak, in terms of 2θ, at about 9.8°, about 6.5°, about 23.4°, about 24.6°, about 16.3°, about 20.4°, and about 19.4°. In yet further embodiments, the crystalline form has an X-ray powder diffraction pattern substantially as shown in FIG. 8, wherein by “substantially” is meant that the reported peaks can vary by about ±0.2°2θ.

In some embodiments, the crystalline form of Compound Ia potassium salt hydrate has a thermogravimetric analysis profile substantially as shown in FIG. 9, wherein by “substantially” is meant that the reported TGA features can vary by about ±5° C., and that that the reported TGA features can vary by about ±2% weight change.

In some embodiments, the crystalline form of Compound Ia potassium salt hydrate has a differential scanning calorimetry thermogram comprising an endotherm with an extrapolated onset temperature between about 135° C. and about 150° C. In some embodiments, the crystalline form has a differential scanning calorimetry thermogram comprising an endotherm with an extrapolated onset temperature at about 142° C. In further embodiments, the crystalline form has a differential scanning calorimetry thermogram substantially as shown in FIG. 10, wherein by “substantially” is meant that the reported DSC features can vary by about ±4° C. and that the reported DSC features can vary by about ±20 joules per gram.

In some embodiments, the crystalline form of Compound Ia potassium salt hydrate has a dynamic moisture sorption (DMS) profile substantially as shown in FIG. 11, wherein by “substantially” is meant that the reported DMS features can vary by about ±5% relative humidity, and that the DMS features reported herein can vary by about ±5% weight change.

### 5. Compound Ia Magnesium Salt Hydrate.

One aspect of the present invention is directed to a crystalline form of Compound Ia magnesium salt hydrate. The physical properties of the crystalline form of Compound Ia magnesium salt hydrate are summarized in Table 9 below.

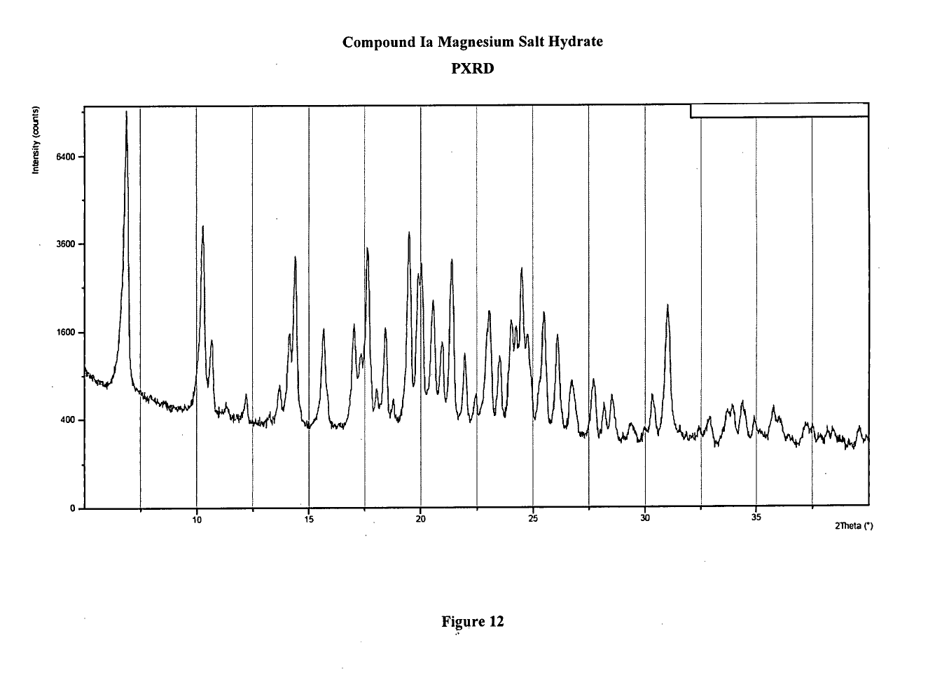

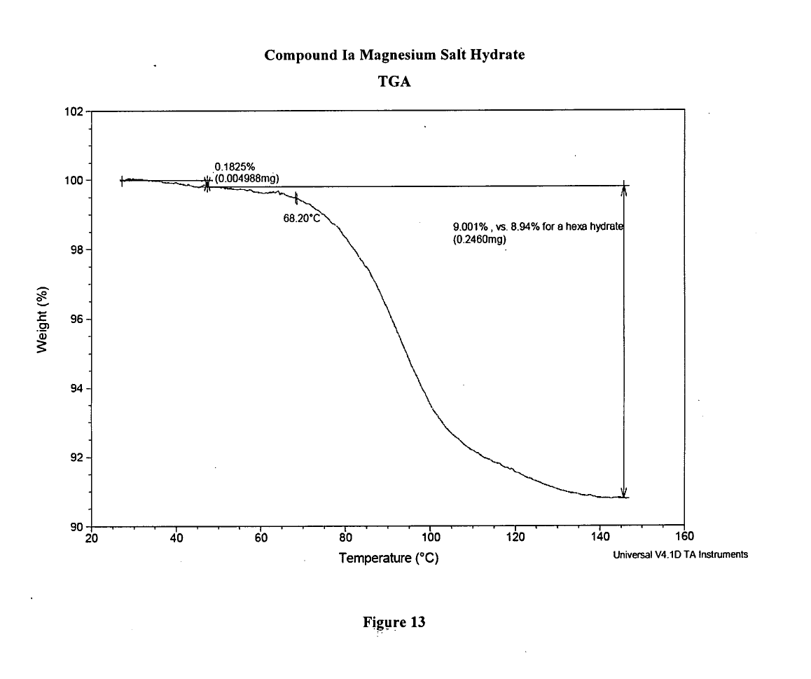

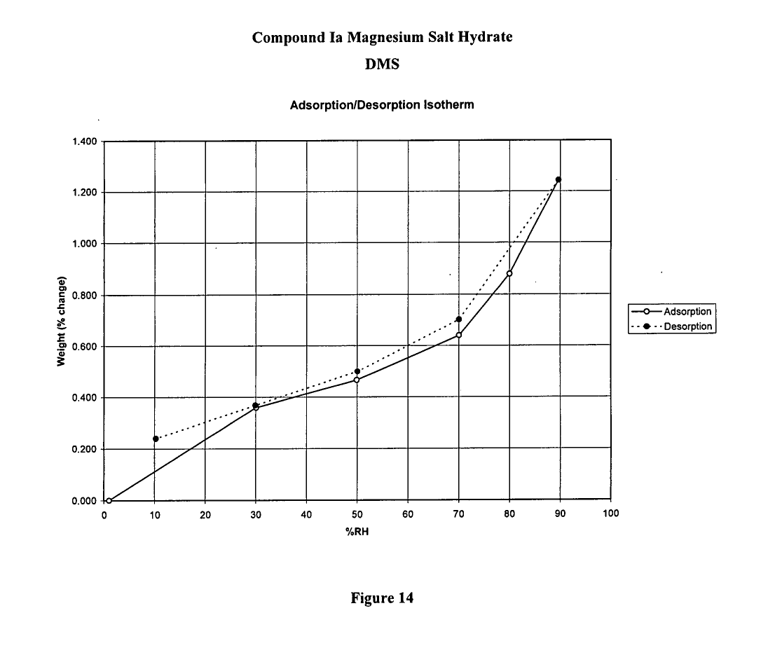

TABLE 9

| | Compound Ia Magnesium Salt Hydrate |
|---|---|
| PXRD | FIG. 12: Peaks of ≧30% relative intensity at 6.9, 10.3, 19.5, |
| | 17.6, 14.4, 21.4, 20.0, 24.5, and 19.9 °2θ |
| TGA | FIG. 13: Decrease in weight of about 9% out to about 150° C. |
| DMS | FIG. 14: Increase of 1.2% weight at about 90% relative humidity |

Certain X-ray powder diffraction peaks for the crystalline form of Compound Ia magnesium salt hydrate are shown in Table 10 below.

TABLE 10

| Pos. [°2θ] | Rel. Int. [%] |
|---|---|
| 6.8702 | 100 |
| 10.2739 | 49.36 |
| 10.6656 | 13.59 |
| 11.316 | 2.11 |
| 12.1975 | 4.16 |
| 13.2325 | 1.56 |
| 13.696 | 5.89 |
| 14.1362 | 16.49 |
| 14.4038 | 39.29 |
| 15.6671 | 17.74 |
| 17.0272 | 18.77 |
| 17.3386 | 11.71 |
| 17.639 | 41.81 |
| 18.0387 | 5.23 |
| 18.4323 | 17.68 |
| 18.7795 | 3.58 |
| 19.4969 | 48.26 |
| 19.9123 | 33.85 |
| 20.0409 | 36.93 |
| 20.5607 | 25.37 |
| 20.961 | 14.58 |
| 21.3993 | 38.22 |
| 21.9727 | 11.96 |
| 22.4585 | 4.47 |
| 22.9333 | 14.37 |
| 23.0666 | 22.74 |
| 23.533 | 11.71 |
| 24.0459 | 20.26 |
| 24.257 | 18.75 |
| 24.5098 | 35.9 |
| 24.757 | 16.83 |
| 25.4955 | 22.45 |
| 26.1037 | 16.89 |
| 26.7303 | 7.11 |
| 27.7196 | 8.24 |
| 28.1878 | 4.13 |
| 28.5321 | 5.66 |
| 29.38 | 1.9 |
| 30.3443 | 5.54 |
| 31.0233 | 25.21 |
| 32.4515 | 1.55 |
| 32.9218 | 2.94 |
| 33.7161 | 3.78 |
| 33.9408 | 4.45 |
| 34.3706 | 5.06 |
| 34.9069 | 3.06 |
| 35.7597 | 4.33 |
| 36.0335 | 2.83 |
| 37.2316 | 2.21 |
| 37.5021 | 2.38 |
| 38.1726 | 1.73 |
| 38.4099 | 1.78 |
| 39.5691 | 1.91 |

TGA showed about 9% weight loss out to 150° C., which corresponds approximately to hexahydrate stoichiometry. The crystalline form of Compound Ia magnesium salt hydrate is slightly hygroscopic up to 90% RH, picking up 1.2% by weight.

One aspect of the present invention is directed to a crystalline form of Compound Ia magnesium salt hydrate having an X-ray powder diffraction pattern comprising a peak, in terms of 2θ, at about 6.9°. In some embodiments, the crystalline form has an X-ray powder diffraction pattern comprising a peak, in terms of 2θ, at about 10.3°. In some embodiments, the crystalline form has an X-ray powder diffraction pattern comprising a peak, in terms of 2θ, at about 6.9° and about 19.5°. In some embodiments, the crystalline form has an X-ray powder diffraction pattern comprising a peak, in terms of 2θ, at about 6.9° and about 10.3°. In some embodiments, the crystalline form has an X-ray powder diffraction pattern comprising a peak, in terms of 2θ, at about 6.9°, about 10.3°, and about 19.5°. In some embodiments, the crystalline form has an X-ray powder diffraction pattern comprising a peak, in terms of 2θ, at about 6.9°, about 10.3°, about 19.5°, about 17.6°, about 14.4°, about 21.4°, and about 20.0°. In some embodiments, the crystalline form has an X-ray powder diffraction pattern comprising a peak, in terms of 2θ, at about 6.9°, about 10.3°, about 19.5°, about 17.6°, about 14.4°, about 21.4°, about 20.0°, about 24.5°, and about 19.9°. In yet further embodiments, the crystalline form has an X-ray powder diffraction pattern substantially as shown in FIG. 12, wherein by “substantially” is meant that the reported peaks can vary by about ±0.2°2θ.

In some embodiments, the crystalline form of Compound Ia magnesium salt hydrate has a thermogravimetric analysis profile substantially as shown in FIG. 13, wherein by “substantially” is meant that the reported TGA features can vary by about ±5° C., and that that the reported TGA features can vary by about ±2% weight change.

In some embodiments, the crystalline form of Compound Ia magnesium salt hydrate has a dynamic moisture sorption (DMS) profile substantially as shown in FIG. 14, wherein by “substantially” is meant that the reported DMS features can vary by about ±5% relative humidity, and that the DMS features reported herein can vary by about ±5% weight change.

### 6. Compound Ia Calcium Salt Hydrate I.

One aspect of the present invention is directed to a crystalline form of Compound Ia calcium salt hydrate I. The physical properties of the crystalline form of Compound Ia calcium salt hydrate I are summarized in Table 11 below.

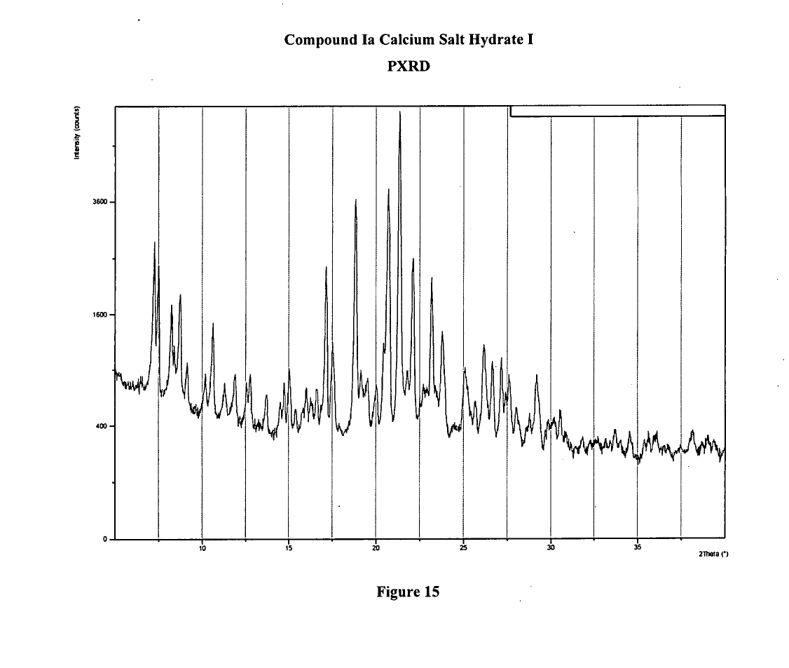

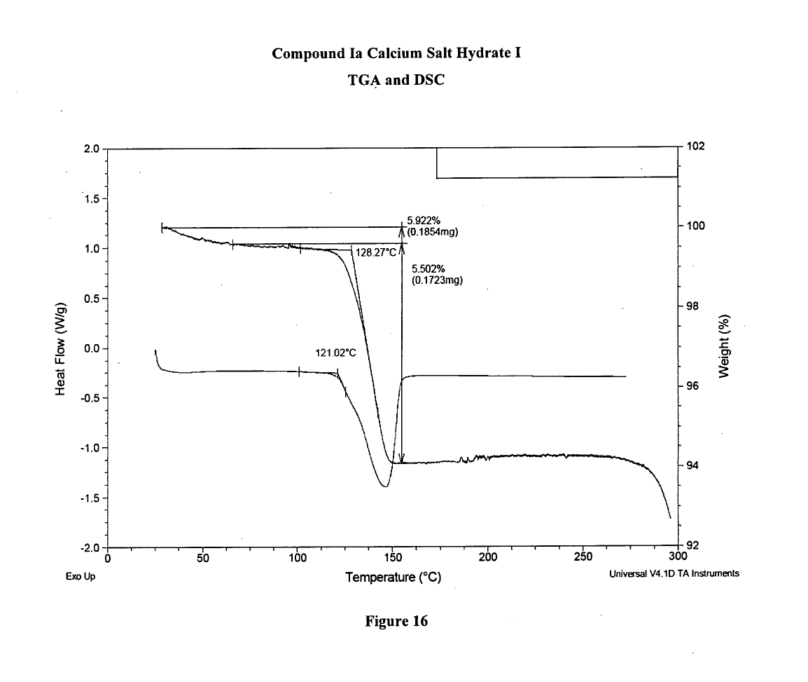

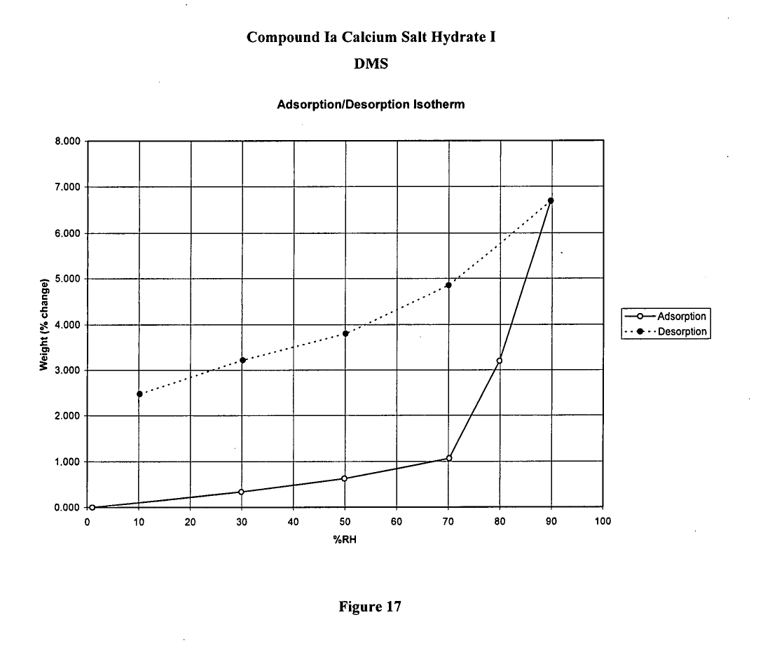

TABLE 11

| | Compound Ia Calcium Salt Hydrate I |
|---|---|
| PXRD | FIG. 15: Peaks of ≧30% relative intensity at 21.3, 20.7, 18.8, |
| | 7.3, 22.1, 17.1, 23.2, and 7.5 °2θ |
| TGA | FIG. 16: Decrease in weight of about 5.9% out to about 160° C. |
| DSC | FIG. 16: Estimated onset temperature for dehydration: about |
| | 121° C. |
| DMS | FIG. 17: Increase of 7% weight at about 90% relative humidity |

Certain X-ray powder diffraction peaks for the crystalline form of Compound Ia calcium salt hydrate I are shown in Table 12 below

TABLE 12

| Pos. [°2θ] | Rel. Int. [%] |
|---|---|
| 6.4745 | 1.62 |
| 7.2605 | 40.31 |
| 7.4967 | 32.19 |
| 8.2485 | 21.91 |
| 8.3984 | 11.32 |
| 8.738 | 25.31 |
| 9.1448 | 8.63 |
| 10.1945 | 7.54 |
| 10.6232 | 18.97 |
| 11.3046 | 6.49 |
| 11.8995 | 8.33 |
| 12.5723 | 7.15 |
| 12.783 | 9.31 |
| 13.7117 | 6.06 |
| 14.4942 | 4.84 |
| 14.7279 | 8.4 |
| 15.0215 | 10.21 |
| 15.3854 | 3.67 |
| 16.0084 | 7.47 |
| 16.2398 | 5.39 |
| 16.6063 | 6.97 |
| 17.1333 | 37.83 |
| 17.5012 | 16.25 |
| 18.8204 | 62.1 |
| 19.1378 | 9.95 |
| 19.5077 | 8.5 |
| 20.0202 | 7.06 |
| 20.4351 | 16.24 |
| 20.697 | 64.58 |
| 21.3397 | 100 |
| 21.7806 | 9.85 |
| 22.1082 | 39.4 |
| 22.7074 | 7.61 |
| 23.1755 | 33.55 |
| 23.7871 | 19.2 |
| 25.0787 | 10.9 |
| 25.6565 | 4.75 |
| 26.1737 | 16.39 |
| 26.6583 | 12.85 |
| 27.165 | 13.63 |
| 27.414 | 6.74 |
| 27.6191 | 9.96 |
| 28.0154 | 4.69 |
| 28.7781 | 4.45 |
| 29.1894 | 11.27 |
| 30.202 | 4.23 |
| 30.5784 | 5.02 |
| 31.7997 | 2.13 |
| 32.6304 | 1.76 |
| 33.6816 | 3.3 |
| 33.9866 | 1.96 |
| 34.5294 | 3 |
| 35.375 | 1.96 |
| 35.6164 | 2.66 |
| 36.0138 | 2.55 |
| 36.6944 | 1.38 |
| 37.4567 | 1.28 |
| 38.1304 | 3.17 |
| 39.012 | 2.35 |

TGA showed about 5.9% weight loss out to 160° C. DSC showed a broad endotherm with an estimated dehydration onset temperature of about 121° C. The crystalline form of Compound Ia calcium salt hydrate I was hygroscopic picking up about 7% by weight at 90% RH and converting to Compound Ia calcium salt hydrate IV during the analysis.

One aspect of the present invention is directed to a crystalline form of Compound Ia calcium salt hydrate I having an X-ray powder diffraction pattern comprising a peak, in terms of 2θ, at about 21.3°. In some embodiments, the crystalline form has an X-ray powder diffraction pattern comprising a peak, in terms of 2θ, at about 20.7°. In some embodiments, the crystalline form has an X-ray powder diffraction pattern comprising a peak, in terms of 2θ, at about 21.3° and about 18.8°. In some embodiments, the crystalline form has an X-ray powder diffraction pattern comprising a peak, in terms of 2θ, at about 21.3° and about 20.7°. In some embodiments, the crystalline form has an X-ray powder diffraction pattern comprising a peak, in terms of 2θ, at about 21.3°, about 20.7°, and about 18.8°. In some embodiments, the crystalline form has an X-ray powder diffraction pattern comprising a peak, in terms of 2θ, at about 21.3°, about 20.7°, about 18.8°, about 7.3°, about 22.1°, about 17.1°, and about 23.2°. In some embodiments, the crystalline form has an X-ray powder diffraction pattern comprising a peak, in terms of 2θ, at about 21.3°, about 20.7°, about 18.8°, about 7.3°, about 22.1°, about 17.1°, about 23.2°, and about 7.5°. In yet further embodiments, the crystalline form has an X-ray powder diffraction pattern substantially as shown in FIG. 15, wherein by “substantially” is meant that the reported peaks can vary by about ±0.2°2θ.

In some embodiments, the crystalline form of Compound Ia calcium salt hydrate I has a thermogravimetric analysis profile substantially as shown in FIG. 16, wherein by “substantially” is meant that the reported TGA features can vary by about ±5° C., and that that the reported TGA features can vary by about ±2% weight change.

In some embodiments, the crystalline form of Compound Ia calcium salt hydrate I has a differential scanning calorimetry thermogram comprising an endotherm with a dehydration onset temperature between about 95° C. and about 145° C. In some embodiments, the crystalline form has a differential scanning calorimetry thermogram comprising an endotherm with a dehydration onset temperature at about 121° C. In further embodiments, the crystalline form has a differential scanning calorimetry thermogram substantially as shown in FIG. 16, wherein by “substantially” is meant that the reported DSC features can vary by about ±25° C. and that the reported DSC features can vary by about ±20 joules per gram.

In some embodiments, the crystalline form of Compound Ia calcium salt hydrate I has a dynamic moisture sorption (DMS) profile substantially as shown in FIG. 17, wherein by “substantially” is meant that the reported DMS features can vary by about ±5% relative humidity, and that the DMS features reported herein can vary by about ±5% weight change.

### 7. Compound Ia Calcium Salt Hydrate II.

One aspect of the present invention is directed to a crystalline form of Compound Ia calcium salt hydrate II. The physical properties of the crystalline form of Compound Ia calcium salt hydrate II are summarized in Table 13 below.

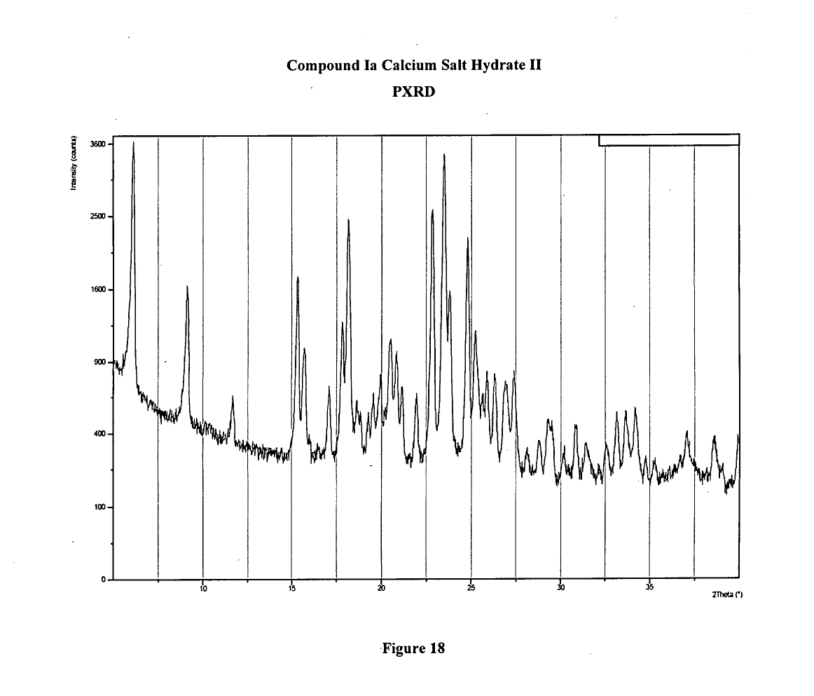

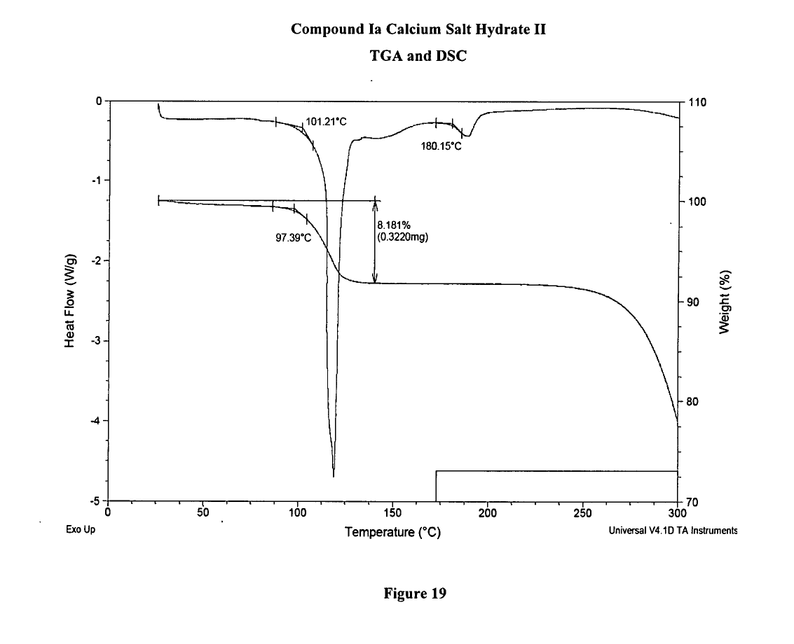

TABLE 13

| | Compound Ia Calcium Salt Hydrate II |
|---|---|
| PXRD | FIG. 18: Peaks of ≧40% relative intensity at 23.5, 6.1, 22.8, |
| | 18.2, 24.8, 15.3, and 23.8 °2θ |
| TGA | FIG. 19: Decrease in weight of about 8.2% out to about 150° C. |
| DSC | FIG. 19: Estimated onset temperature for dehydration: about |
| | 101° C. |

Certain X-ray powder diffraction peaks for the crystalline form of Compound Ia calcium salt hydrate II are shown in Table 14 below.

TABLE 14

| Pos. [°2θ] | Rel. Int. [%] |
|---|---|
| 6.1221 | 92.52 |
| 9.1348 | 37.48 |
| 10.3712 | 1.86 |
| 11.6884 | 8.38 |
| 15.3186 | 46.89 |
| 15.6833 | 23.96 |
| 16.4569 | 2.36 |
| 17.092 | 13.85 |
| 17.822 | 31.82 |
| 18.1585 | 69.32 |
| 18.6326 | 11.06 |
| 18.853 | 8.37 |
| 19.2722 | 7.83 |
| 19.5604 | 13.13 |
| 19.9594 | 17.03 |
| 20.4431 | 24.07 |
| 20.5482 | 24.98 |
| 20.8311 | 22.41 |
| 21.152 | 14.54 |
| 21.9604 | 12.29 |
| 22.8429 | 74.19 |
| 23.4958 | 100 |
| 23.8016 | 42.69 |
| 24.8016 | 62.8 |
| 25.2417 | 29.7 |
| 25.6334 | 12.89 |
| 25.878 | 19.5 |
| 26.3144 | 18.52 |
| 26.8223 | 14.68 |
| 26.9277 | 16.43 |
| 27.3861 | 19.42 |
| 28.1169 | 4.25 |
| 28.8049 | 5.57 |
| 29.2876 | 9.01 |
| 29.5092 | 8.32 |
| 30.1813 | 3.79 |
| 30.8398 | 8.54 |
| 31.4194 | 5.61 |
| 32.1456 | 2.1 |
| 32.553 | 5.45 |
| 33.1339 | 11.53 |
| 33.6408 | 10.79 |
| 34.1639 | 11.49 |
| 34.7504 | 3.77 |
| 35.2489 | 3.04 |
| 36.6762 | 4.11 |
| 37.0485 | 7.54 |
| 38.5842 | 6.75 |
| 39.033 | 2.81 |

TGA showed about 8.2% weight loss out to 150° C. DSC showed an endotherm with an estimated dehydration onset temperature of about 101° C.

One aspect of the present invention is directed to a crystalline form of Compound Ia calcium salt hydrate II having an X-ray powder diffraction pattern comprising a peak, in terms of 2θ, at about 23.5°. In some embodiments, the crystalline form has an X-ray powder diffraction pattern comprising a peak, in terms of 2θ, at about 6.1°. In some embodiments, the crystalline form has an X-ray powder diffraction pattern comprising a peak, in terms of 2θ, at about 23.5° and about 22.8°. In some embodiments, the crystalline form has an X-ray powder diffraction pattern comprising a peak, in terms of 2θ, at about 23.5° and about 6.1°. In some embodiments, the crystalline form has an X-ray powder diffraction pattern comprising a peak, in terms of 2θ, at about 23.5°, about 6.1°, and about 22.8°. In some embodiments, the crystalline form has an X-ray powder diffraction pattern comprising a peak, in terms of 2θ, at about 23.5°, about 6.1°, about 22.8°, about 18.2°, about 24.8°, about 15.3°, and about 23.8°. In yet further embodiments, the crystalline form has an X-ray powder diffraction pattern substantially as shown in FIG. 18, wherein by “substantially” is meant that the reported peaks can vary by about ±0.2°2θ.

In some embodiments, the crystalline form of Compound Ia calcium salt hydrate II has a thermogravimetric analysis profile substantially as shown in FIG. 19, wherein by “substantially” is meant that the reported TGA features can vary by about ±5° C., and that that the reported TGA features can vary by about ±2% weight change.

In some embodiments, the crystalline form of Compound Ia calcium salt hydrate II has a differential scanning calorimetry thermogram comprising an endotherm with a dehydration onset temperature between about 95° C. and about 110° C. In some embodiments, the crystalline form has a differential scanning calorimetry thermogram comprising an endotherm with an extrapolated onset temperature at about 101° C. In further embodiments, the crystalline form has a differential scanning calorimetry thermogram substantially as shown in FIG. 19, wherein by “substantially” is meant that the reported DSC features can vary by about ±25° C. and that the reported DSC features can vary by about ±20 joules per gram.

### 8. Compound Ia Calcium Salt Hydrate III.

One aspect of the present invention is directed to a crystalline form of Compound Ia calcium salt hydrate III. The physical properties of the crystalline form of Compound Ia calcium salt hydrate III are summarized in Table 15 below.

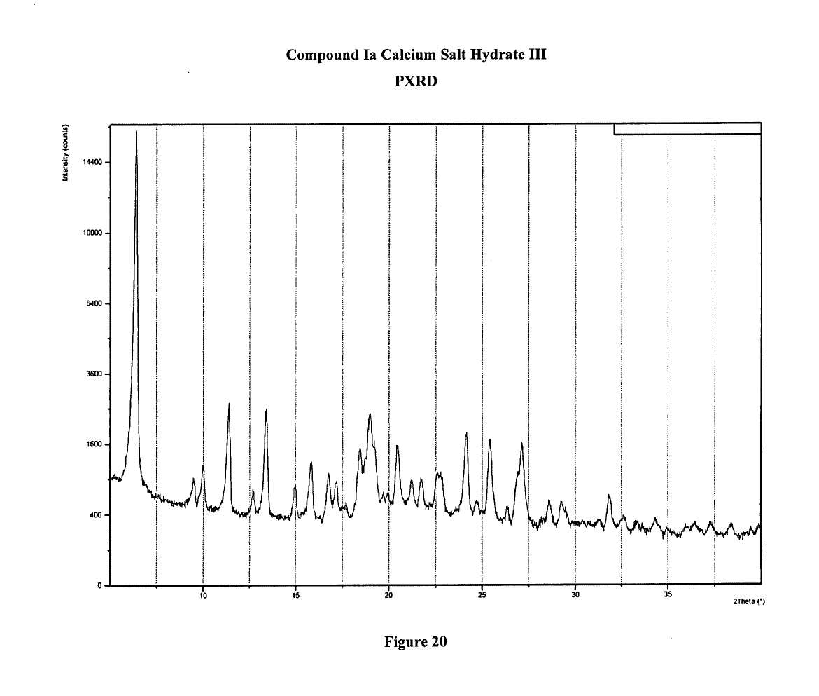

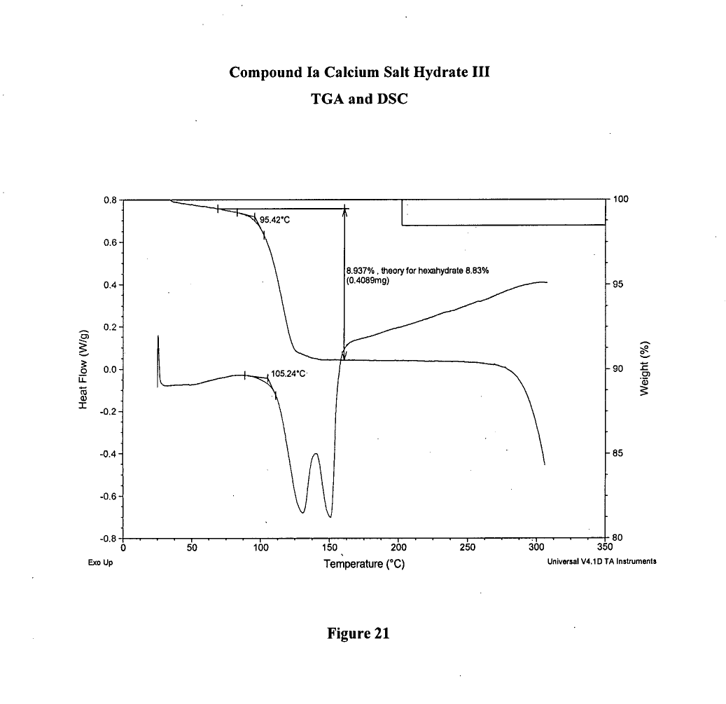

TABLE 15

| | Compound Ia Calcium Salt Hydrate III |
|---|---|
| PXRD | FIG. 20: Peaks of ≧8% relative intensity at 6.4, 11.4, 13.4, |
| | 19.0, 24.1, 25.4, and 27.1 °2θ |
| TGA | FIG. 21: Decrease in weight of about 8.9% out to about 160° C. |
| DSC | FIG. 21: Estimated onset temperature for dehydration: about |
| | 105° C. |

Certain X-ray powder diffraction peaks for the crystalline form of Compound Ia calcium salt hydrate III are shown in Table 16 below.

TABLE 16

| Pos. [°2θ] | Rel. Int. [%] |
|---|---|
| 6.3953 | 100 |
| 9.4775 | 2.74 |
| 9.999 | 4.21 |
| 11.3854 | 13.93 |
| 12.7038 | 2.03 |
| 13.4011 | 13.34 |
| 14.9499 | 2.68 |
| 15.8225 | 5.51 |
| 16.7605 | 4.04 |
| 17.1677 | 3.11 |
| 17.6845 | 1.09 |
| 18.4497 | 7.16 |
| 18.9605 | 12.33 |
| 19.2497 | 6.97 |
| 19.6859 | 1.85 |
| 19.9366 | 1.94 |
| 20.4521 | 7.49 |
| 21.2122 | 3.09 |
| 21.7215 | 3.31 |
| 22.6 | 4.21 |
| 22.8303 | 3.61 |
| 24.1487 | 9.75 |
| 24.7079 | 1.6 |
| 25.4065 | 8.76 |
| 26.3486 | 1.45 |
| 26.8412 | 4.02 |
| 27.1243 | 8.65 |
| 28.6071 | 2.2 |
| 29.2637 | 2.13 |
| 30.7363 | 0.65 |
| 31.2784 | 0.82 |
| 31.8445 | 2.62 |
| 32.6388 | 1.08 |
| 33.3277 | 0.76 |
| 34.3226 | 1.09 |
| 34.96 | 0.5 |
| 36.4432 | 0.83 |
| 37.2994 | 0.78 |
| 38.4072 | 0.78 |
| 39.4563 | 0.5 |

TGA showed about 8.9% weight loss out to 160° C. DSC showed an endotherm with an estimated dehydration onset temperature of about 105° C.

One aspect of the present invention is directed to a crystalline form of Compound Ia calcium salt hydrate III having an X-ray powder diffraction pattern comprising a peak, in terms of 2θ, at about 6.4°. In some embodiments, the crystalline form has an X-ray powder diffraction pattern comprising a peak, in terms of 2θ, at about 11.4°. In some embodiments, the crystalline form has an X-ray powder diffraction pattern comprising a peak, in terms of 2θ, at about 6.4° and about 13.4°. In some embodiments, the crystalline form has an X-ray powder diffraction pattern comprising a peak, in terms of 2θ, at about 6.4° and about 11.4°. In some embodiments, the crystalline form has an X-ray powder diffraction pattern comprising a peak, in terms of 2θ, at about 6.4°, about 11.4°, and about 13.4°. In some embodiments, the crystalline form has an X-ray powder diffraction pattern comprising a peak, in terms of 2θ, at about 6.4°, about 11.4°, about 13.4°, about 19.0°, about 24.1°, about 25.4°, and about 27.1°. In yet further embodiments, the crystalline form has an X-ray powder diffraction pattern substantially as shown in FIG. 20, wherein by “substantially” is meant that the reported peaks can vary by about ±0.2°2θ.

In some embodiments, the crystalline form of Compound Ia calcium salt hydrate III has a thermogravimetric analysis profile substantially as shown in FIG. 21, wherein by “substantially” is meant that the reported TGA features can vary by about ±5° C., and that that the reported TGA features can vary by about ±2% weight change.

In some embodiments, the crystalline form of Compound Ia calcium salt hydrate III has a differential scanning calorimetry thermogram comprising an endotherm with a dehydration onset temperature between about 95° C. and about 115° C. In some embodiments, the crystalline form has a differential scanning calorimetry thermogram comprising an endotherm with a dehydration onset temperature at about 105° C. In further embodiments, the crystalline form has a differential scanning calorimetry thermogram substantially as shown in FIG. 21, wherein by “substantially” is meant that the reported DSC features can vary by about ±25° C. and that the reported DSC features can vary by about ±20 joules per gram.

### 9. Compound Ia Calcium Salt Hydrate IV.

One aspect of the present invention is directed to a crystalline form of Compound Ia calcium salt hydrate IV. The physical properties of the crystalline form of Compound Ia calcium salt hydrate IV are summarized in Table 17 below.

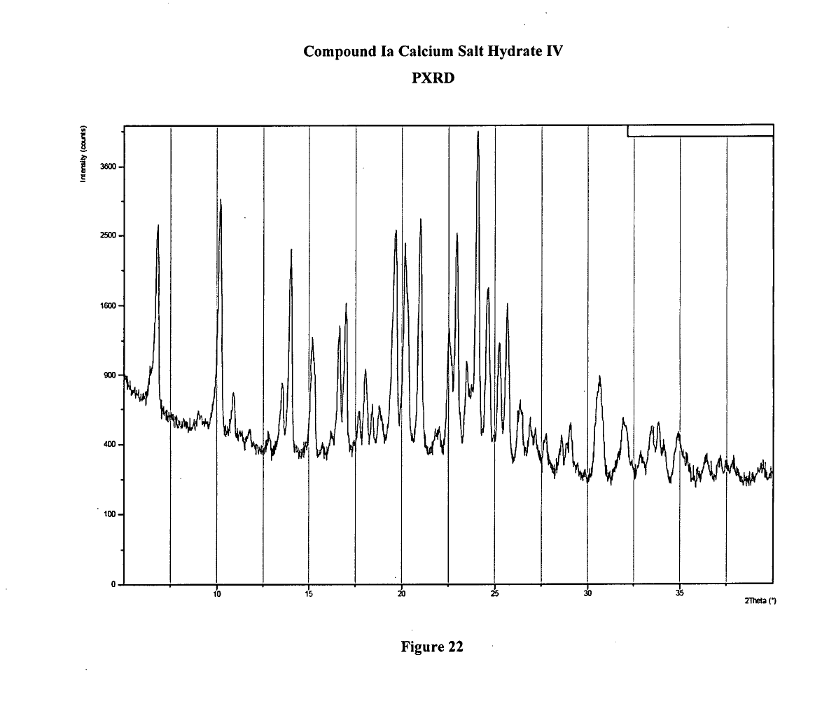

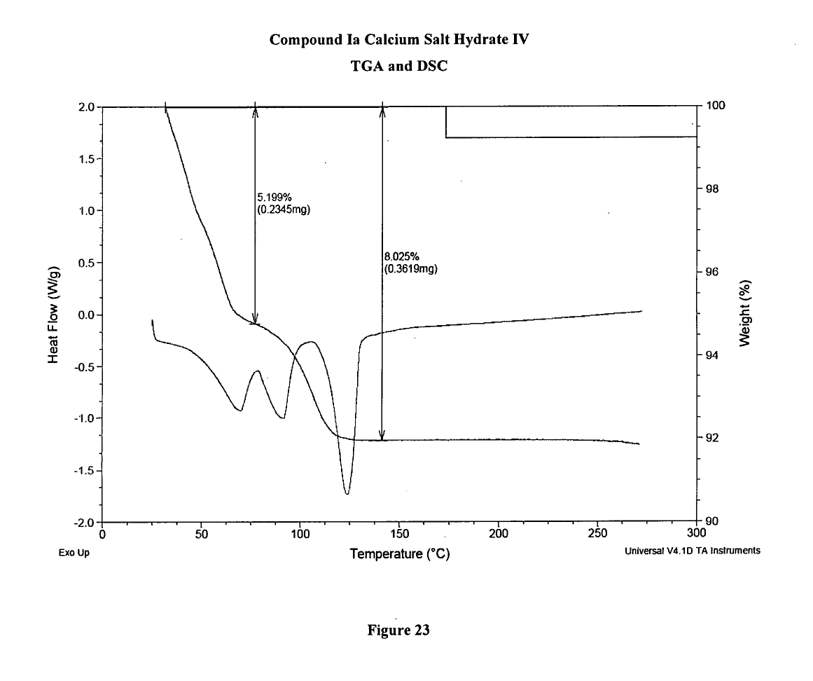

TABLE 17

| | Compound Ia Calcium Salt Hydrate IV |
|---|---|
| PXRD | FIG. 22: Peaks of ≧50% relative intensity at 24.1, 10.2, 21.0, |
| | 19.7, 22.9, 6.8, 14.0, and 20.2 and °2θ |
| TGA | FIG. 23: Decrease in weight of about 8% out to about 150° C. |
| DSC | FIG. 23: Estimated onset temperature for dehydration: about |
| | 110° C. |

Certain X-ray powder diffraction peaks for the crystalline form of Compound Ia calcium salt hydrate IV are shown in Table 18 below.

TABLE 18

| Pos. [°2θ] | Rel. Int. [%] |
|---|---|
| 6.8189 | 51.61 |
| 9.0685 | 3.23 |
| 10.1868 | 65.22 |
| 10.8999 | 9.4 |
| 11.7758 | 2.91 |
| 12.8341 | 3.02 |
| 13.547 | 13.09 |
| 14.0099 | 51.18 |
| 15.1789 | 23.75 |
| 16.2121 | 3.44 |
| 16.6292 | 26.76 |
| 16.9894 | 33.1 |
| 17.6937 | 7.43 |
| 18.0421 | 15.8 |
| 18.4104 | 8.6 |
| 18.7956 | 7.76 |
| 19.6537 | 56.87 |
| 20.1701 | 50.23 |
| 20.3313 | 29.79 |
| 20.9912 | 61.5 |
| 21.9209 | 3.95 |
| 22.5318 | 26.24 |
| 22.9427 | 55.91 |
| 23.4899 | 18.22 |
| 24.0625 | 100 |
| 24.6268 | 39.43 |
| 25.2394 | 23.25 |
| 25.6697 | 33.75 |
| 26.3591 | 10.52 |
| 26.8972 | 7.31 |
| 27.1947 | 5.77 |
| 27.73 | 5.16 |
| 28.6038 | 5.7 |
| 29.0713 | 7.03 |
| 30.4736 | 12.45 |
| 30.6584 | 17.69 |
| 31.9332 | 8.7 |
| 32.1248 | 7.19 |
| 32.8932 | 3.32 |
| 33.4707 | 7.71 |
| 33.822 | 8.28 |
| 34.1122 | 5.02 |
| 34.8991 | 6.5 |
| 36.4045 | 3.34 |
| 37.1264 | 2.7 |
| 37.8447 | 2.63 |
| 39.4105 | 2.16 |

TGA showed about 8% weight loss out to 150° C. DSC showed an endotherm with an estimated dehydration onset temperature of about 110° C.

One aspect of the present invention is directed to a crystalline form of Compound Ia calcium salt hydrate IV having an X-ray powder diffraction pattern comprising a peak, in terms of 2θ, at about 24.1°. In some embodiments, the crystalline form has an X-ray powder diffraction pattern comprising a peak, in terms of 2θ, at about 10.2°. In some embodiments, the crystalline form has an X-ray powder diffraction pattern comprising a peak, in terms of 2θ, at about 24.1° and about 21.0°. In some embodiments, the crystalline form has an X-ray powder diffraction pattern comprising a peak, in terms of 2θ, at about 24.1° and about 10.2°. In some embodiments, the crystalline form has an X-ray powder diffraction pattern comprising a peak, in terms of 2θ, at about 24.1°, about 10.2°, and about 21.0°. In some embodiments, the crystalline form has an X-ray powder diffraction pattern comprising a peak, in terms of 2θ, at about 24.1°, about 10.2°, about 21.0°, about 19.7°, about 22.9°, about 6.8°, about 14.0°, and about 20.2°. In yet further embodiments, the crystalline form has an X-ray powder diffraction pattern substantially as shown, in FIG. 22, wherein by “substantially” is meant that the reported peaks can vary by about ±0.2°2θ.

In some embodiments, the crystalline form of Compound Ia calcium salt hydrate IV has a thermogravimetric analysis profile substantially as shown in FIG. 23, wherein by “substantially” is meant that the reported TGA features can vary by about ±5° C., and that that the reported TGA features can vary by about ±2% weight change.

In some embodiments, the crystalline form of Compound Ia calcium salt hydrate IV has a differential scanning calorimetry thermogram comprising an endotherm with a dehydration onset temperature between about 95° C. and about 135° C. In some embodiments, the crystalline form has a differential scanning calorimetry thermogram comprising an endotherm with a dehydration onset temperature at about 110° C. In further embodiments, the crystalline form has a differential scanning calorimetry thermogram substantially as shown in FIG. 23, wherein by “substantially” is meant that the reported DSC features can vary by about ±25° C.

### 10. Compound Ia TRIS Salt.

One aspect of the present invention is directed to a crystalline form of Compound Ia TRIS salt. The physical properties of the crystalline form of Compound Ia TRIS salt are summarized in Table 19 below.

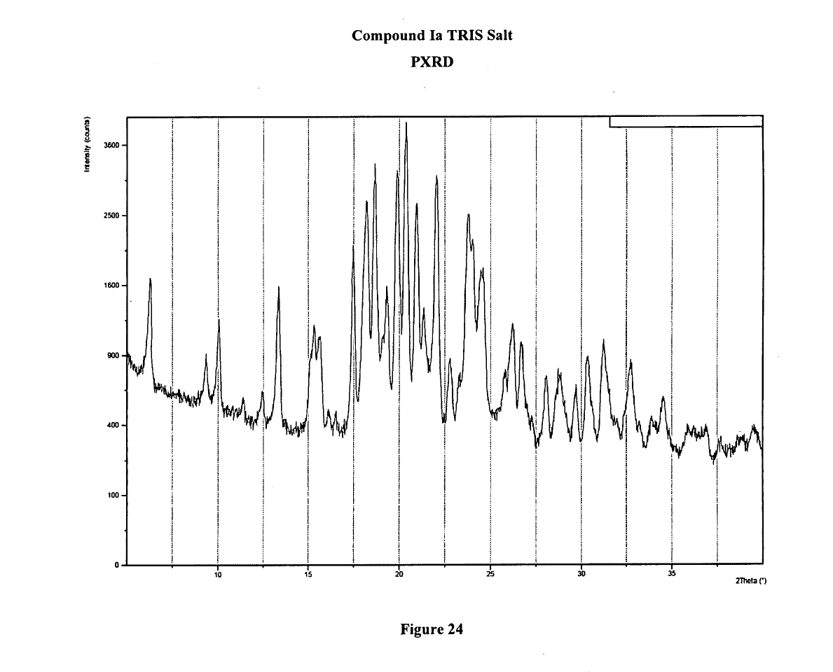

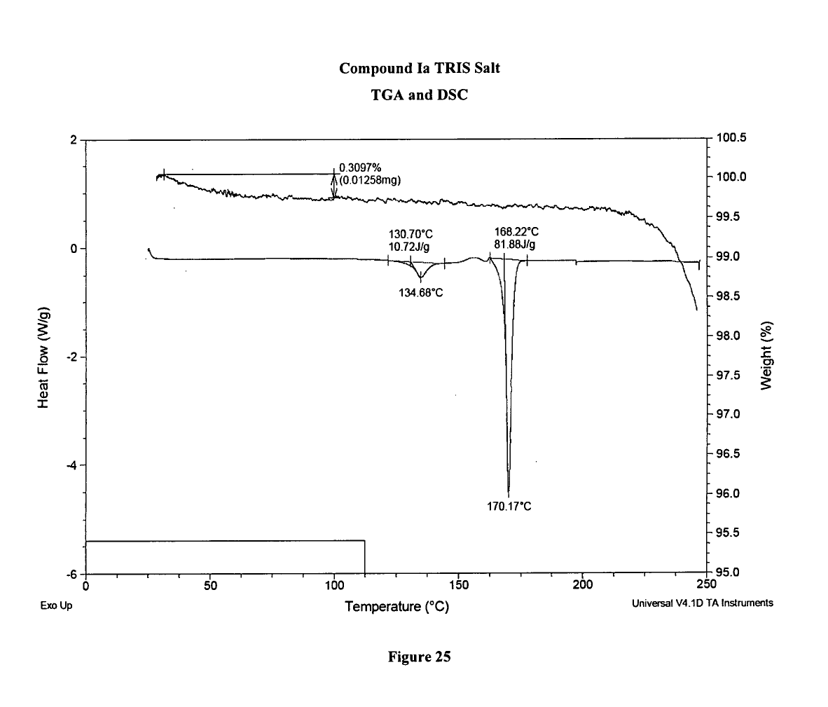

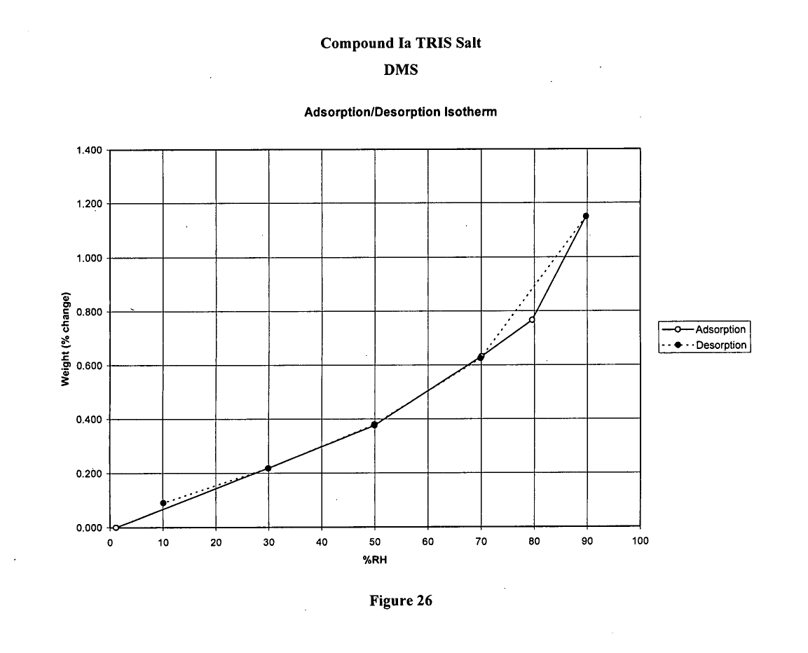

TABLE 19

| | Compound Ia TRIS Salt |
|---|---|
| PXRD | FIG. 24: Peaks of ≧40% relative intensity at 20.4, 18.7, 19.9, |
| | 22.0, 18.2, 20.9, 23.8, 24.0, 17.5, and 24.6 °2θ |
| TGA | FIG. 25: Decrease in weight of about 0.3% out to about 100° C. |
| DSC | FIG. 25: Small broad endotherm with extrapolated onset |
| | temperature: about 131° C. Apparent melting endotherm with |
| | extrapolated onset temperature: about 168° C. |
| DMS | FIG. 26: Increase of about 1.2% weight at about 90% relative |
| | humidity |

Certain X-ray powder diffraction peaks for the crystalline form of Compound Ia TRIS salt are shown in Table 20 below.

TABLE 20

| Pos. [°2θ] | Rel. Int. [%] |
|---|---|
| 6.2755 | 26.43 |
| 9.3465 | 11.33 |
| 10.0544 | 21.71 |
| 11.3976 | 3.82 |
| 12.4611 | 5.79 |
| 13.3492 | 33.43 |
| 15.0698 | 14.15 |
| 15.3159 | 22.98 |
| 15.6023 | 19.82 |
| 16.123 | 3.86 |
| 16.4992 | 3.86 |
| 17.4707 | 46.21 |
| 18.2054 | 64.81 |
| 18.6659 | 80.19 |
| 19.3065 | 33.84 |
| 19.887 | 77.92 |
| 20.3681 | 100 |
| 20.9428 | 63.39 |
| 21.3447 | 25.3 |
| 22.0408 | 74.23 |
| 22.7714 | 13.82 |
| 23.2505 | 9.64 |
| 23.7944 | 58.96 |
| 24.0159 | 49.96 |
| 24.4199 | 36.51 |
| 24.5913 | 40.81 |
| 25.7975 | 12.03 |
| 26.2202 | 24.53 |
| 26.6872 | 19.79 |
| 27.2733 | 4.64 |
| 28.0664 | 12.13 |
| 28.7944 | 11.89 |
| 29.6787 | 8.72 |
| 30.2932 | 16.08 |
| 31.2333 | 19.8 |
| 32.7423 | 14.51 |
| 33.8537 | 4.95 |
| 34.5297 | 8.53 |
| 35.9 | 4.01 |
| 36.8936 | 4.34 |
| 37.6573 | 2.28 |
| 38.9311 | 2.94 |

TGA showed about 0.3% weight loss out to 100° C. indicating an anhydrous form. DSC showed a small, broad endotherm with an extrapolated onset temperature of about 131° C., and an apparent melting endotherm with an extrapolated onset temperature of about 168° C. The crystalline form of Compound Ia TRIS salt is slightly hygroscopic up to 90% RH, picking up about 1.2% by weight.

One aspect of the present invention is directed to a crystalline form of Compound Ia TRIS salt having an X-ray powder diffraction pattern comprising a peak, in terms of 2θ, at about 20.4°. In some embodiments, the crystalline form has an X-ray powder diffraction pattern comprising a peak, in terms of 2θ, at about 18.7°. In some embodiments, the crystalline form has an X-ray powder diffraction pattern comprising a peak, in terms of 2θ, at about 20.4° and about 19.9°. In some embodiments, the crystalline form has an X-ray powder diffraction pattern comprising a peak, in terms of 2θ, at about 20.4° and about 18.7°. In some embodiments, the crystalline form has an X-ray powder diffraction pattern comprising a peak, in terms of 2θ, at about 20.4°, about 18.7°, and about 19.9°. In some embodiments, the crystalline form has an X-ray powder diffraction pattern comprising a peak, in terms of 2θ, at about 20.4°, about 18.7°, about 19.9°, about 22.0°, about 18.2°, about 20.9°, and about 23.8°. In some embodiments, the crystalline form has an X-ray powder diffraction pattern comprising a peak, in terms of 2θ, at about 20.4°, about 18.7°, about 19.9°, about 22.0°, about 18.2°, about 20.9°, about 23.8°, about 24.0°, about 17.5°, and about 24.6°. In yet further embodiments, the crystalline form has an X-ray powder diffraction pattern substantially as shown in FIG. 24, wherein by “substantially” is meant that the reported peaks can vary by about ±0.2°2θ.

In some embodiments, the crystalline form of Compound Ia TRIS salt has a thermogravimetric analysis profile substantially as shown in FIG. 25, wherein by “substantially” is meant that the reported TGA features can vary by about ±5° C., and that that the reported TGA features can vary by about ±2% weight change.

In some embodiments, the crystalline form of Compound Ia TRIS salt has a differential scanning calorimetry thermogram comprising an endotherm with an extrapolated onset temperature between about 160° C. and about 175° C. In some embodiments, the crystalline form has a differential scanning calorimetry thermogram comprising an endotherm with an extrapolated onset temperature at about 168° C. In further embodiments, the crystalline form has a differential scanning calorimetry thermogram substantially as shown in FIG. 25, wherein by “substantially” is meant that the reported DSC features can vary by about ±25° C. and that the reported DSC features can vary by about ±20 joules per gram.

In some embodiments, the crystalline form of Compound Ia TRIS salt has a dynamic moisture sorption (DMS) profile substantially as shown in FIG. 25, wherein by “substantially” is meant that the reported DMS features can vary by about ±5% relative humidity, and that the DMS features reported herein can vary by about ±5% weight change.

### 11. Compound Ia TRIS Salt Hydrate.

One aspect of the present invention is directed to a crystalline form of Compound Ia TRIS salt hydrate. The physical properties of the crystalline form of Compound Ia TRIS salt hydrate are summarized in Table 21 below.

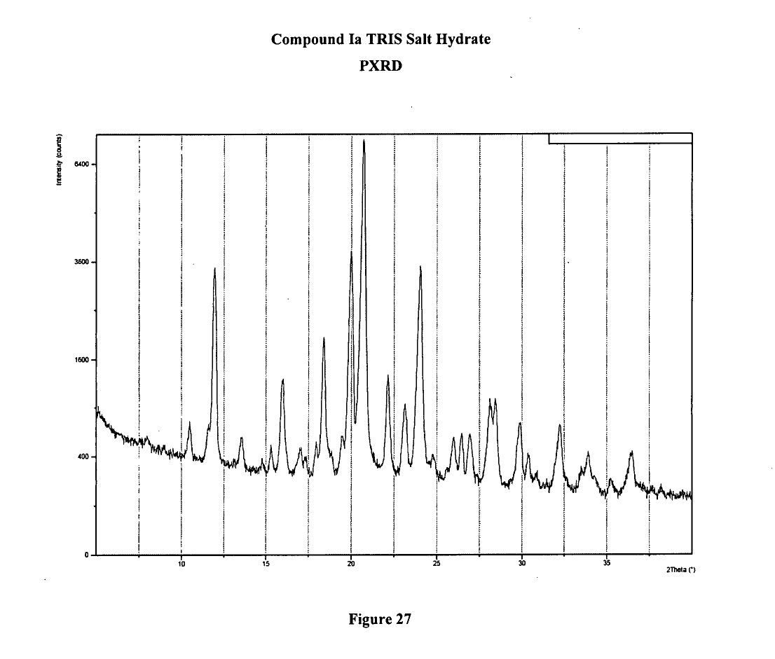

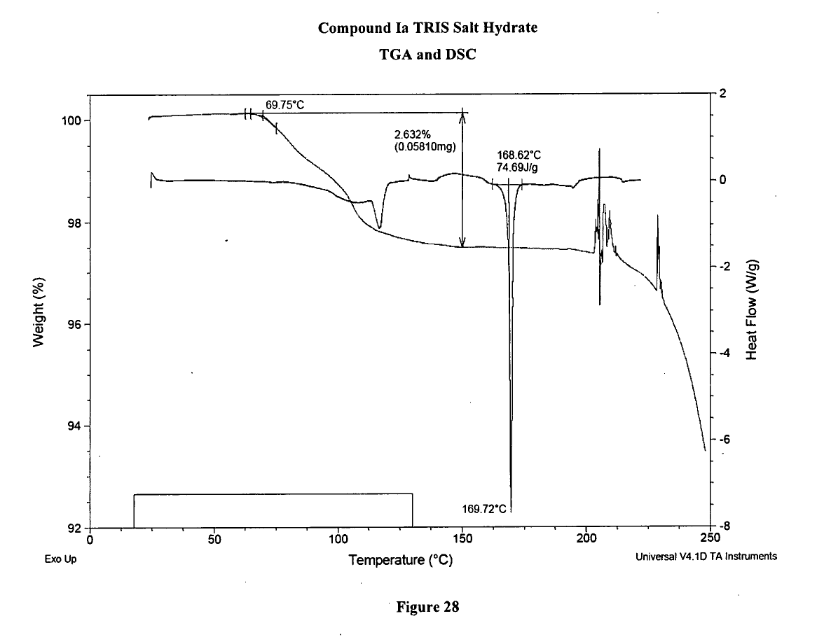

TABLE 21

| | Compound Ia TRIS Salt Hydrate |
|---|---|
| PXRD | FIG. 27: Peaks of ≧14% relative intensity at 20.7, |
| | 20.0, 24.0, 12.0, 18.4, 22.1, and 16.0 °2θ |
| TGA | FIG. 28: Decrease in weight of about 2.6% out to |
| | about 150° C. |
| DSC | FIG. 28: Post-dehydration endotherm extrapolated |
| | onset temperature: about 169° C. |

Certain X-ray powder diffraction peaks for the crystalline form of Compound Ia TRIS salt hydrate are shown in Table 22 below.

TABLE 22

| Pos. [°2θ] | Rel. Int. [%] |
|---|---|
| 7.9806 | 1.62 |
| 10.4911 | 5.13 |
| 11.5927 | 4.86 |
| 11.9548 | 44.69 |
| 13.5746 | 4.17 |
| 14.7821 | 1.55 |
| 15.2931 | 3.12 |
| 15.9815 | 14.58 |
| 17.0116 | 2.92 |
| 17.3121 | 1.83 |
| 17.9469 | 3.81 |
| 18.377 | 24.61 |
| 19.4631 | 4.71 |
| 19.9882 | 51.73 |
| 20.7125 | 100 |
| 22.1456 | 15.54 |
| 23.1356 | 10.06 |
| 24.04 | 46.08 |
| 24.7801 | 2.65 |
| 25.9928 | 5.11 |
| 26.4564 | 5.7 |
| 26.9443 | 5.71 |
| 28.1174 | 11.32 |
| 28.4348 | 11.61 |
| 29.8813 | 7.66 |
| 30.3725 | 3.27 |
| 30.8395 | 1.26 |
| 32.2302 | 7.53 |
| 33.515 | 2.13 |
| 33.9094 | 3.95 |
| 35.2077 | 1.38 |
| 36.3867 | 4.16 |
| 37.6437 | 0.71 |
| 38.1923 | 0.66 |

TGA showed about 2.6% weight loss out to 150° C., which corresponds approximately to monohydrate stoichiometry. DSC showed an endotherm with an extrapolated onset temperature of about 169° C., which is consistent with the melting endotherm observed for the anhydrous TRIS salt of Compound Ia.

One aspect of the present invention is directed to a crystalline form of Compound Ia TRIS salt hydrate having an X-ray powder diffraction pattern comprising a peak, in terms of 2θ, at about 20.7°. In some embodiments, the crystalline form has an X-ray powder diffraction pattern comprising a peak, in terms of 2θ, at about 20.0°. In some embodiments, the crystalline form has an X-ray powder diffraction pattern comprising a peak, in terms of 2θ, at about 20.7° and about 24.0°. In some embodiments, the crystalline form has an X-ray powder diffraction pattern comprising a peak, in terms of 2θ, at about 20.7° and about 20.0°. In some embodiments, the crystalline form has an X-ray powder diffraction pattern comprising a peak, in terms of 2θ, at about 20.7°, about 20.0°, and about 24.0°. In some embodiments, the crystalline form has an X-ray powder diffraction pattern comprising a peak, in terms of 2θ, at about 20.7°, about 20.0°, about 24.0°, about 12.0°, about 18.4°, about 22.1°, and about 16.0°. In yet further embodiments, the crystalline form has an X-ray powder diffraction pattern substantially as shown in FIG. 27, wherein by “substantially” is meant that the reported peaks can vary by about ±0.2°2θ.

In some embodiments, the crystalline form of Compound Ia TRIS salt hydrate has a thermogravimetric analysis profile substantially as shown in FIG. 28, wherein by “substantially” is meant that the reported TGA features can vary by about ±5° C., and that that the reported TGA features can vary by about ±2% weight change.

In some embodiments, the crystalline form of Compound Ia TRIS salt hydrate has a differential scanning calorimetry thermogram comprising an endotherm with an extrapolated onset temperature between about 160° C. and about 175° C. In some embodiments, the crystalline form has a differential scanning calorimetry thermogram comprising an endotherm with an extrapolated onset temperature at about 169° C. In further embodiments, the crystalline form has a differential scanning calorimetry thermogram substantially as shown in FIG. 28, wherein by “substantially” is meant that the reported DSC features can vary by about ±4° C. and that the reported DSC features can vary by about ±20 joules per gram.

### 12. Compound Ia L-Arginine Salt Hydrate.

One aspect of the present invention is directed to a crystalline form of Compound Ia L-arginine salt hydrate. The physical properties of the crystalline form of Compound Ia L-arginine salt hydrate are summarized in Table 23 below.

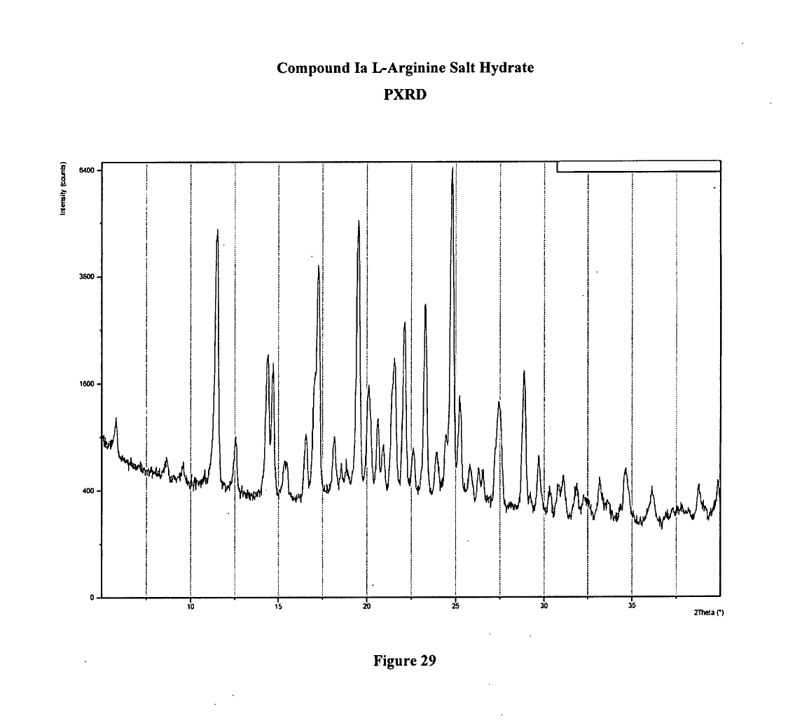

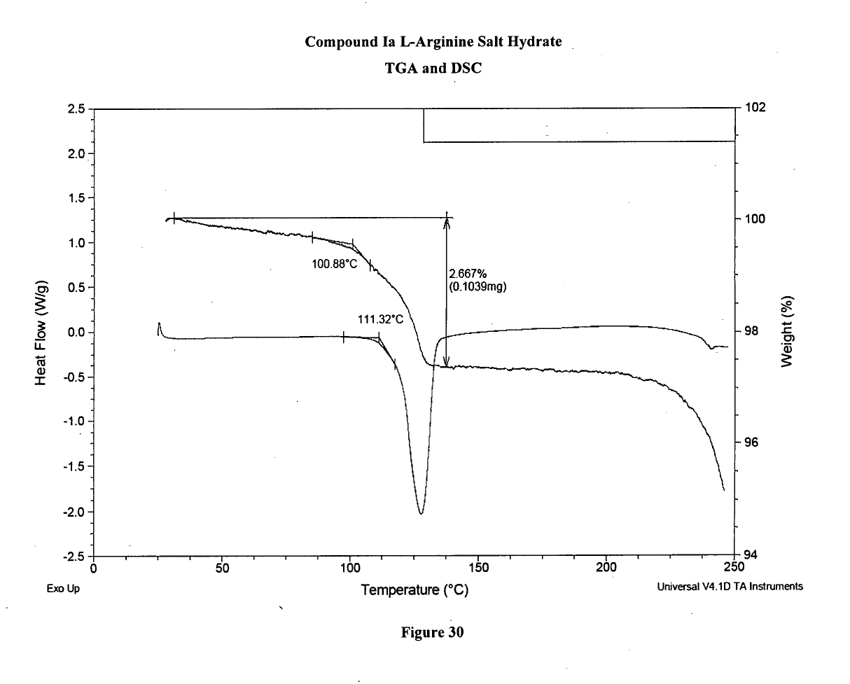

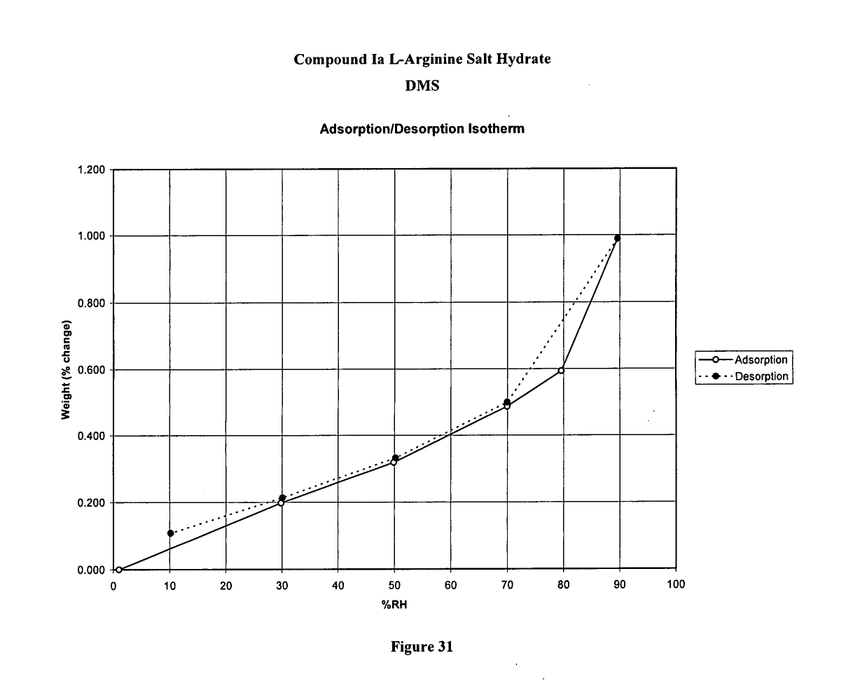

TABLE 23

| | Compound Ia L-Arginine Salt Hydrate |
|---|---|
| PXRD | FIG. 29: Peaks of ≧ relative intensity at 24.8, 19.5, 11.5, 17.3, |
| | 23.3, 22.1, and 14.4 °2θ |
| TGA | FIG. 30: Decrease in weight of about 2.7% out to about 140° C. |
| DSC | FIG. 30: Estimated onset temperature for dehydration: about |
| | 111° C. |
| DMS | FIG. 31: Increase of about 1% weight at about 90% relative |
| | humidity |

Certain X-ray powder diffraction peaks for the crystalline form of Compound Ia L-arginine salt hydrate are shown in Table 24 below

TABLE 24

| Pos. [°2θ] | Rel. Int. [%] |
|---|---|
| 5.8058 | 6.81 |
| 8.6686 | 2.96 |
| 9.5768 | 2.58 |
| 11.5196 | 71.41 |
| 12.5751 | 8.54 |
| 14.3917 | 28 |
| 14.6854 | 25.75 |
| 15.3612 | 5.05 |
| 15.4658 | 4.8 |
| 16.5636 | 9.88 |
| 17.017 | 20.76 |
| 17.2639 | 57.49 |
| 18.1693 | 9.41 |
| 18.5553 | 4.31 |
| 18.8429 | 4.58 |
| 19.5164 | 76.36 |
| 20.1025 | 19.98 |
| 20.6222 | 12.64 |
| 20.9266 | 7.56 |
| 21.4135 | 17.41 |
| 21.5602 | 27.08 |
| 22.1233 | 37.64 |
| 22.6261 | 7.66 |
| 23.3038 | 44.47 |
| 23.934 | 6.68 |
| 24.4672 | 10.46 |
| 24.8054 | 100 |
| 25.2423 | 17.85 |
| 25.8228 | 5.39 |
| 26.3061 | 5.16 |
| 26.5407 | 4.68 |
| 27.4409 | 17.32 |
| 28.8794 | 25.53 |
| 29.2187 | 2.39 |
| 29.7079 | 7.92 |
| 30.3296 | 2.93 |
| 30.8192 | 3.73 |
| 31.0979 | 4.77 |
| 31.8549 | 3.97 |
| 32.2769 | 2.39 |
| 33.1611 | 4.52 |
| 33.6342 | 2.02 |
| 34.625 | 6.28 |
| 36.1252 | 3.83 |
| 37.6705 | 1.51 |
| 38.7765 | 4.04 |

TGA showed about 2.7% weight loss out to 140° C., which corresponds approximately to monohydrate stoichiometry. DSC showed an endotherm with an estimated dehydration onset temperature of about 111° C. The crystalline form of Compound Ia L-arginine salt hydrate is slightly hygroscopic picking up about 1% by weight at 90% RH.

One aspect of the present invention is directed to a crystalline form of Compound Ia L-arginine salt hydrate having an X-ray powder diffraction pattern comprising a peak, in terms of 2θ, at about 24.8°. In some embodiments, the crystalline form has an X-ray powder diffraction pattern comprising a peak, in terms of 2θ, at about 19.5°. In some embodiments, the crystalline form has an X-ray powder diffraction pattern comprising a peak, in terms of 2θ, at about 24.8° and about 11.5°. In some embodiments, the crystalline form has an X-ray powder diffraction pattern comprising a peak, in terms of 2θ, at about 24.8° and about 19.5°. In some embodiments, the crystalline form has an X-ray powder diffraction pattern comprising a peak, in terms of 2θ, at about 24.8°, about 19.5°, and about 11.5°. In some embodiments, the crystalline form has an X-ray powder diffraction pattern comprising a peak, in terms of 2θ, at about 24.8°, about 19.5°, about 11.5°, about 17.3°, about 23.3°, about 22.1°, and about 14.4°. In yet further embodiments, the crystalline form has an X-ray powder diffraction pattern substantially as shown in FIG. 29, wherein by “substantially” is meant that the reported peaks can vary by about ±0.2°2θ.

In some embodiments, the crystalline form of Compound Ia L-arginine salt hydrate has a thermogravimetric analysis profile substantially as shown in FIG. 30, wherein by “substantially” is meant that the reported TGA features can vary by about ±5° C., and that that the reported TGA features can vary by about ±2% weight change.

In some embodiments, the crystalline form of Compound Ia L-arginine salt hydrate has a differential scanning calorimetry thermogram comprising an endotherm with a dehydration onset temperature between about 90° C. and about 130° C. In some embodiments, the crystalline form has a differential scanning calorimetry thermogram comprising an endotherm with a dehydration onset temperature at about 111° C. In further embodiments, the crystalline form has a differential scanning calorimetry thermogram substantially as shown in FIG. 30, wherein by “substantially” is meant that the reported DSC features can vary by about ±25° C. and that the reported DSC features can vary by about ±20 joules per gram.

In some embodiments, the crystalline form of Compound Ia L-arginine salt hydrate has a dynamic moisture sorption (DMS) profile substantially as shown in FIG. 31, wherein by “substantially” is meant that the reported DMS features can vary by about ±5% relative humidity, and that the DMS features reported herein can vary by about ±5% weight change.

The crystalline forms described herein can be prepared by any of the suitable procedures known in the art for preparing crystalline polymorphs. In some embodiments the crystalline forms described herein are prepared according to the Examples. In some embodiments, the crystalline forms described herein can be prepared by heating crystalline forms other than the crystalline forms described herein. In some embodiments, the crystalline forms described herein can be prepared by recrystallizing crystalline forms other than the crystalline forms described herein.

### 13. Compositions Containing Crystalline Forms of the Present Invention

The present invention further provides compositions containing a crystalline form of Compound I, or a salt, solvate or hydrate thereof, described herein.

The present invention further provides compositions containing a crystalline form of Compound Ia, or a salt, solvate or hydrate thereof, described herein.

In some embodiments, the compositions of the invention include at least about 1, about 5, about 10, about 20, about 30, or about 40% by weight of a crystalline form of Compound Ia, or a salt, solvate or hydrate thereof.

In some embodiments, the compositions of the invention include at least about 50, about 60, about 70, about 80, about 90, about 95, about 96, about 97, about 98, or about 99% by weight of a crystalline form of Compound Ia, or a salt, solvate or hydrate thereof.

In some embodiments, compositions of the invention include of a crystalline form of Compound Ia, or a salt, solvate or hydrate thereof and a pharmaceutically acceptable carrier.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All subcombinations of the salts, solvates, hydrates and crystalline forms specifically exemplified herein, as well as all subcombinations of uses thereof and medical indications related thereto described herein, are also specifically embraced by the present invention just as if each and every subcombination of salts, solvates, hydrates and crystalline forms specifically exemplified herein and subcombination of uses thereof and medical indications related thereto was individually and explicitly recited herein.

It is understood that each embodiment that pertains to salts, solvates, hydrates, or crystalline forms of the present invention may be optionally provided in combination with one or more of provisos that relate to:
2-(2-((4-(((4-chlorophenyl)(phenyl)carbamoyloxy)methyl)cyclohexyl)methoxy)acetamido)ethanesulfonic acid (Compound I free acid hydrate);
a crystalline form of 2-(2-((4-(((4-chlorophenyl)(phenyl)carbamoyloxy)methyl)cyclohexyl)methoxy)acetamido)ethanesulfonic acid (Compound I free acid hydrate);
2-(2-(((1r,4r)-4-(((4-chlorophenyl)(phenyl)carbamoyloxy)methyl)cyclohexyl)methoxy)acetamido)ethanesulfonic acid (Compound Ia free acid hydrate);
a crystalline form of 2-(2-(((1r,4r)-4-(((4-chlorophenyl)(phenyl)carbamoyloxy)methyl)cyclohexyl)methoxy)acetamido)ethanesulfonic acid (Compound Ia free acid hydrate);
sodium 2-(2-(((1r,4r)-4-(((4-chlorophenyl)(phenyl)carbamoyloxy)methyl)cyclohexyl)methoxy)acetamido)ethanesulfonate (Compound Ia sodium salt);
sodium 2-(2-(((1r,4r)-4-(((4-chlorophenyl)(phenyl)carbamoyloxy)methyl)cyclohexyl)methoxy)acetamido)ethanesulfonate (Compound Ia sodium salt hydrate I);
sodium 2-(2-(((1r,4r)-4-(((4-chlorophenyl)(phenyl)carbamoyloxy)methyl)cyclohexyl)methoxy)acetamido)ethanesulfonate (Compound Ia sodium salt hydrate II);
a crystalline form of sodium 2-(2-(((1r,4r)-4-(((4-chlorophenyl)(phenyl)carbamoyloxy)methyl)cyclohexyl)methoxy)acetamido)ethanesulfonate (Compound Ia sodium salt);
a crystalline form of 2-(2-(((1r,4r)-4-(((4-chlorophenyl)(phenyl)carbamoyloxy)methyl)cyclohexyl)methoxy)acetamido)ethanesulfonic acid hydrate (Compound Ia hydrate I); and
a crystalline form of sodium 2-(2-(((1r,4r)-4-(((4-chlorophenyl)(phenyl)carbamoyloxy)methyl)cyclohexyl)methoxy)acetamido)ethanesulfonate Compound Ia sodium salt hydrate II).

It is further understood that each embodiment that pertains to processes of the present invention may be optionally provided in combination with one or more of the provisos that relate to:

in a process for preparing a salt of the invention, the cation is other than sodium;

in a process for activating a compound of Formula IV, if the activating agent is thionyl chloride, then R¹ is other than H;

in a process for activating a compound of Formula IV, if the activating agent is 1H-benzo[d][1,2,3]triazol-1-ol, and R¹ is sodium, then the reacting of a compound of Formula IV with an activating agent is carried out in the presence of a substituted carbodiimide;

in a process for activating a compound of Formula IV, Y is other than Cl; and

in a process for activating a compound of Formula IV, Y is other than 1H-benzo[d][1,2,3]triazol-1-yloxy.

### Isotopes

The present disclosure includes all isotopes of atoms occurring in the present compounds, intermediates, salts, solvates, hydrates, and crystalline forms. Isotopes include those atoms having the same atomic number but different mass numbers. One aspect of the present invention includes every combination of one or more atoms in the present compounds, intermediates, salts, solvates, hydrates, and crystalline forms that is replaced with an atom having the same atomic number but a different mass number. One such example is the replacement of an atom that is the most naturally abundant isotope, such as ¹H or ¹²C, found in one the present compounds, intermediates, salts, solvates, hydrates, and crystalline forms, with a different atom that is not the most naturally abundant isotope, such as ²H or ³H (replacing ¹H), or ¹¹C, ¹³C, or ¹⁴C (replacing ¹²C). A compound wherein such a replacement has taken place is commonly referred to as being an isotopically-labeled compound. Isotopic-labeling of the present compounds, intermediates, salts, solvates, hydrates, and crystalline forms can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸C. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as, those prepared during synthesis, preformulation, and the like, and pharmaceutical compositions, such as, those prepared with the intent of using in a mammal for the treatment of one or more of the disorders described herein, comprising one or more of the present compounds, intermediates, salts, solvates, hydrates, and crystalline forms, wherein the naturally occurring distribution of the isotopes in the composition is perturbed. Another aspect of the present invention includes compositions and pharmaceutical compositions comprising compounds, intermediates, salts, solvates, hydrates, and crystalline forms as described herein wherein the compound, intermediate, salt, solvate, hydrate, or crystalline form is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

Indications and Methods of Prophylaxis and/or Treatment

In addition to the foregoing beneficial uses for the modulators of PGI2 receptor activity disclosed herein, the compositions disclosed herein are useful in the treatment of several additional diseases and disorders, and in the amelioration of symptoms thereof. Without limitation, these include the following:

1. Pulmonary Arterial Hypertension (PAH)

Pulmonary arterial hypertension (PAH) has a multifactorial pathobiology. Vasoconstriction, remodeling of the pulmonary vessel wall, and thrombosis contribute to increased pulmonary vascular resistance in PAH (Humbert et al., J. Am. Coll. Cardiol., 2004, 43:13 S-24S.)

The pharmaceutical compositions of the present invention disclosed herein are useful in the treatment of pulmonary arterial hypertension (PAH) and symptoms thereof. PAH shall be understood to encompass the following forms of pulmonary arterial hypertension described in the 2003 World Health Organization (WHO) clinical classification of pulmonary arterial hypertension: idiopathic PAH (IPAH); familial PAH (FPAH); PAH associated with other conditions (APAH), such as PAH associated with collagen vascular disease, PAH associated with congenital systemic-to-pulmonary shunts, PAH associated with portal hypertension, PAH associated with HIV infection, PAH associated with drugs or toxins, or PAH associated with Other; and PAH associated with significant venous or capillary involvement.

Idiopathic PAH refers to PAH of undetermined cause.

Familial PAH refers to PAH for which hereditary transmission is suspected or documented.

PAH associated with collagen vascular disease shall be understood to encompass PAH associated with scleroderma, PAH associated with CREST (calcinosis cutis, Raynaud's phenomenon, esophageal dysfunction, sclerodactyl), and telangiectasias) syndrome, PAH associated with systemic lupus erythematosus (SLE), PAH associated with rheumatoid arthritis, PAH associated with Takayasu's arteritis, PAH associated with polymyositis, and PAH associated with dermatomyositis.

PAH associated with congenital systemic-to-pulmonary shunts shall be understood to encompass PAH associated with atrial septic defect (ASD), PAH associated with ventricular septic defect (VSD) and PAH associated with patent ductus arteriosus.

PAH associated with drugs or toxins shall be understood to encompass PAH associated with ingestion of aminorex, PAH associated with ingestion of a fenfluramine compound (e.g., PAH associated with ingestion of fenfluramine or PAH associated with ingestion of dexfenfluramine), PAH associated with ingestion of certain toxic oils (e.g., PAH associated with ingestion of rapeseed oil), PAH associated with ingestion of pyrrolizidine alkaloids (e.g., PAH associated with ingestion of bush tea) and PAH associated with ingestion of monocrotaline.

PAH associated with Other shall be understood to encompass PAH associated with a thyroid disorder, PAH associated with glycogen storage disease, PAH associated with Gaucher disease, PAH associated with hereditary hemorrhagic telangiectasia, PAH associated with a hemoglobinopathy, PAH associated with a myeloproliferative disorder, and PAH associated with splenectomy.

PAH associated with significant venous or capillary involvement shall be understood to encompass PAH associated with pulmonary veno-occlusive disease (PVOD) and PAH associated with pulmonary capillary hemangiomatosis (PCH).

(See, e.g., Simonneau et al., J. Am. Coll. Cardiol., 2004, 43:5 S-12S; McGoon et al., Chest, 2004, 126:14 S-34S; Rabinovitch, Annu. Rev. Pathol. Mech. Dis., 2007, 2:369-399; McLaughlin et al., Circulation, 2006, 114:1417-1431; Strauss et al., Clin. Chest. Med., 2007, 28:127-142; Taichman et al., Clin. Chest. Med., 2007, 28:1-22.)

Evidence for the association of PAH with scleroderma and the beneficial effect of an agonist of the PGI2 receptor on PAH is given by Badesch et al. (Badesch et al., Ann. Intern. Med., 2000, 132:425-434). Evidence for the association of PAH with the collagen vascular diseases mixed connective tissue disease (MCTD), systemic lupus erythematosus (SLE), Sjögren's syndrome and CREST syndrome and the beneficial effect of an agonist of the PGI2 receptor on PAH is given by Humbert et al. (Eur. Respir. J., 1999, 13:1351-1356). Evidence for the association of PAH with CREST syndrome and the beneficial effect of an agonist of the PGI2 receptor on PAH is given by Miwa et al. (Int. Heart J., 2007, 48:417-422). Evidence for the association of PAH with SLE and the beneficial effect of an agonist of the PGI2 receptor on PAH is given by Robbins et al. (Chest, 2000, 117:14-18). Evidence for the association of PAH with HIV infection and the beneficial of an agonist of the PGI2 receptor on PAH is given by Aguilar et al. (Am. J. Respir. Crit. Care Med., 2000, 162:1846-1850). Evidence for the association of PAH with congenital heart defects (including ASD, VSD and patent ductus arteriosus) and the beneficial effect of an agonist of the PGI2 receptor on PAH is given by Rosenzweig et al. (Circulation, 1999, 99:1858-1865). Evidence for the association of PAH with fenfluramine and with dexfenfluramine, anorexigens, is given by Archer et al. (Am. J. Respir. Crit. Care Med., 1998, 158:1061-1067). Evidence for the association of PAH with hereditary hemorrhagic telangiectasia is given by McGoon et al. (Chest, 2004, 126:14-34). Evidence for the association of PAH with splenectomy is given by Hoeper et al. (Ann. Intern. Med., 1999, 130:506-509). Evidence for the association of PAH with portal hypertension and the beneficial effect of an agonist of the PGI2 receptor on PAH is given by Hoeper et al. (Eur. Respir. J., 2005, 25:502-508).

Symptoms of PAH include dyspnea, angina, syncope and edema (McLaughlin et al., Circulation, 2006, 114:1417-1431). The pharmaceutical compositions of the present invention disclosed herein are useful in the treatment of symptoms of PAH.

2. Antiplatelet Therapies (Conditions Related to Platelet Aggregation)

Antiplatelet agents (antiplatelets) are prescribed for a variety of conditions. For example, in coronary artery disease they are used to help prevent myocardial infarction or stroke in patients who are at risk of developing obstructive blood clots (e.g., coronary thrombosis).

In a myocardial infarction (“MI” or “heart attack”), the heart muscle does not receive enough oxygen-rich blood as a result of a blockage in the coronary blood vessels. If taken while an attack is in progress or immediately afterward (preferably within 30 min), antiplatelets can reduce the damage to the heart.

A transient ischemic attack (“TIA” or “mini-stroke”) is a brief interruption of oxygen flow to the brain due to decreased blood flow through arteries, usually due to an obstructing blood clot. Antiplatelet drugs have been found to be effective in preventing TIAs.

Angina is a temporary and often recurring chest pain, pressure or discomfort caused by inadequate oxygen-rich blood flow (ischemia) to some parts of the heart. In patients with angina, antiplatelet therapy can reduce the effects of angina and the risk of myocardial infarction.

Stroke is an event in which the brain does not receive enough oxygen-rich blood, usually due to blockage of a cerebral blood vessel by a blood clot. In high-risk patients, taking antiplatelets regularly has been found to prevent the formation of blood clots that cause first or second strokes.

Angioplasty is a catheter based technique used to open arteries obstructed by a blood clot. Whether or not stenting is performed immediately after this procedure to keep the artery open, antiplatelets can reduce the risk of forming additional blood clots following the procedure(s).

Coronary bypass surgery is a surgical procedure in which an artery or vein is taken from elsewhere in the body and grafted to a blocked coronary artery, rerouting blood around the blockage and through the newly attached vessel. After the procedure, antiplatelets can reduce the risk of secondary blood clots.

Atrial fibrillation is the most common type of sustained irregular heart rhythm (arrhythmia). Atrial fibrillation affects about two million Americans every year. In atrial fibrillation, the atria (the heart's upper chambers) rapidly fire electrical signals that cause them to quiver rather than contract normally. The result is an abnormally fast and highly irregular heartbeat. When given after an episode of atrial fibrillation, antiplatelets can reduce the risk of blood clots forming in the heart and traveling to the brain (embolism).

There is evidence that a PGI2 receptor agonist will inhibit platelet aggregation and thus be a potential treatment as an antiplatelet therapy (see, e.g., Moncada et al., Lancet, 1977, 1:18-20). It has been shown that genetic deficiency of the PGI2 receptor in mice leads to an increased propensity towards thrombosis (Murata et al., Nature, 1997, 388:678-682).

PGI2 receptor agonists can be used to treat, for example, claudication or peripheral artery disease as well as cardiovascular complications, arterial thrombosis, atherosclerosis, vasoconstriction caused by serotonin, ischemia-reperfusion injury, and restenosis of arteries following angioplasty or stent placement. (See, e.g., Fetalvero et al., Prostaglandins Other Lipid Mediat., 2007, 82:109-118; Arehart et al., Curr. Med. Chem., 2007, 14:2161-2169; Davi et al., N. Engl. J. Med., 2007, 357:2482-2494; Fetalvero et al., Am. J. Physiol. Heart. Circ. Physiol., 2006, 290:H1337-H1346; Murata et al., Nature, 1997, 388:678-682; Wang et al., Proc. Natl. Acad. Sci. USA, 2006, 103:14507-14512; Xiao et al., Circulation, 2001, 104:2210-2215; McCormick et al., Biochem. Soc. Trans., 2007, 35:910-911; Arehart et al., Circ. Res., 2008, Mar. 6 Epub ahead of print.)

PGI2 receptor agonists can also be used alone or in combination with thrombolytic therapy, for example, tissue-type plasminogen activator (t-PA), to provide cardioprotection following MI or postischemic myocardial dysfunction or protection from ischemic injury during percutaneous coronary intervention, and the like, including complications resulting therefrom. PGI2 receptor agonists can also be used in antiplatelet therapies in combination with, for example, alpha-tocopherol (vitamin E), echistatin (a disintegrin) or, in states of hypercoagulability, heparin. (See, e.g., Chan., J. Nutr., 1998, 128:1593-1596; Mardla et al., Platelets, 2004, 15:319-324; Bernabei et al., Ann. Thorac. Surg., 1995, 59:149-153; Gainza et al., J. Nephrol., 2006, 19:648-655.)

The PGI2 receptor agonists disclosed herein provide beneficial improvement in microcirculation to patients in need of antiplatelet therapy by antagonizing the vasoconstrictive products of the aggregating platelets in, for example and not limited to the indications described above. Accordingly, in some embodiments, the present invention provides methods for reducing platelet aggregation in a patient in need thereof, comprising administering to the patient a composition comprising a PGI2 receptor agonist disclosed herein. In further embodiments, the present invention provides methods for treating coronary artery disease, myocardial infarction, transient ischemic attack, angina, stroke, atrial fibrillation, or a symptom of any of the foregoing in a patient in need of the treatment, comprising administering to the patient a composition comprising a PGI2 receptor agonist disclosed herein.

In further embodiments, the present invention provides methods for reducing risk of blood clot formation in an angioplasty or coronary bypass surgery patient, or a patient suffering from atrial fibrillation, comprising administering to the patient a composition comprising a PGI2 receptor agonist disclosed herein at a time where such risk exists.

3. Atherosclerosis

Atherosclerosis is a complex disease characterized by inflammation, lipid accumulation, cell death and fibrosis. It is the leading cause of mortality in many countries, including the United States. Atherosclerosis, as the term is used herein, shall be understood to encompass disorders of large and medium-sized arteries that result in the progressive accumulation within the intima of smooth muscle cells and lipids.

It has been shown that an agonist of the PGI2 receptor can confer protection from atherosclerosis, such as from atherothrombosis (Arehart et al., Curr. Med. Chem., 2007, 14:2161-2169; Stitham et al., Prostaglandins Other Lipid Mediat., 2007, 82:95-108; Fries et al., Hematology Am. Soc. Hematol. Educ. Program, 2005, :445-451; Egan et al., Science, 2004, 306:1954-1957; Kobayashi et al., J. Clin. Invest., 2004, 114:784-794; Arehart et al., Circ. Res., 2008, Mar. 6 Epub ahead of print).

It has been shown that defective PGI2 receptor signaling appears to accelerate atherothrombosis in humans, i.e. that an agonist of the PGI2 receptor can confer protection from atherothrombosis in humans (Arehart et al., Circ. Res., 2008, Mar. 6 Epub ahead of print).

The pharmaceutical compositions of the present invention disclosed herein are useful in the treatment of atherosclerosis, and the treatment of the symptoms thereof. Accordingly, in some embodiments, the present invention provides methods for treating atherosclerosis in a patient in need of the treatment, comprising administering to the patient a composition comprising a PGI2 receptor agonist disclosed herein. In further embodiments, methods are provided for treating a symptom of atherosclerosis in a patient in need of the treatment, comprising administering to the patient a composition comprising a PGI2 receptor agonist disclosed herein.

4. Asthma

Asthma is a lymphocyte-mediated inflammatory airway disorder characterized by airway eosinophilia, increased mucus production by goblet cells, and structural remodeling of the airway wall. The prevalence of asthma has dramatically increased worldwide in recent decades. It has been shown that genetic deficiency of the PGI2 receptor in mice augments allergic airway inflammation (Takahashi et al., Br J Pharmacol, 2002, 137:315-322). It has been shown that an agonist of the PGI2 receptor can suppress not only the development of asthma when given during the sensitization phase, but also the cardinal features of experimental asthma when given during the challenge phase (Idzko et al., J. Clin. Invest., 2007, 117:464-472; Nagao et al., Am. J. Respir. Cell Mol. Biol., 2003, 29:314-320), at least in part through markedly interfering with the function of antigen-presenting dendritic cells within the airways (Idzko et al., J. Clin. Invest., 2007, 117:464-472; Zhou et al., J. Immunol., 2007, 178:702-710; Jaffar et al., J. Immunol., 2007, 179:6193-6203; Jozefowski et al., Immunopharmacol., 2003, 3:865-878). These cells are crucial for both the initiation and the maintenance phases of allergic asthma, as depletion of airway dendritic cells during secondary challenge in sensitized mice abolished all characteristic features of asthma, an effect that could be completely restored by adoptive transfer of wild-type dendritic cells (van Rijt et al., J. Exp. Med., 2005, 201:981-991). It has also been shown that an agonist of the PGI2 receptor can inhibit proinflammatory cytokine secretion by human alveolar macrophages (Raychaudhuri et al., J. Biol. Chem., 2002, 277:33344-33348). The pharmaceutical compositions of the present invention disclosed herein are useful in the treatment of asthma, and the treatment of the symptoms thereof. Accordingly, in some embodiments, the present invention provides methods for treating asthma in a patient in need of the treatment, comprising administering to the patient a composition comprising a PGI2 receptor agonist disclosed herein. In further embodiments, methods are provided for treating a symptom of asthma in a patient in need of the treatment, comprising administering to the patient a composition comprising a PGI2 receptor agonist disclosed herein.

5. Diabetic-Related Pathologies

Although hyperglycemia is the major cause for the pathogenesis of diabetic complications such as diabetic peripheral neuropathy (DPN), diabetic nephropathy (DN) and diabetic retinopathy (DR), enhanced vasoconstriction and platelet aggregation in diabetic patients has also been implicated to play a role in disease progression (Cameron et al., Naunyn Schmiedebergs Arch. Pharmacol., 2003, 367:607-614). Agonists of the PGI2 receptor promote vasodilation and inhibit platelet aggregation. Improving microvascular blood flow is able to benefit diabetic complications (Cameron, Diabetologia, 2001, 44:1973-1988).

It has been shown that an agonist of the PGI2 receptor can prevent and reverse motor and sensory peripheral nerve conduction abnormalities in streptozotocin-diabetic rats (Cotter et Naunyn Schmiedebergs Arch. Pharmacol., 1993, 347:534-540). Further evidence for the beneficial effect of an agonist of the PGI2 receptor in the treatment of diabetic peripheral neuropathy is given by Hotta et al. (Diabetes, 1996, 45:361-366), Ueno et al. (Jpn. J. Pharmacol., 1996, 70:177-182), Ueno et al. (Life Sci., 1996, 59:PL105-PL110), Hotta et al. (Prostaglandins, 1995, 49:339-349), Shindo et al. (Prostaglandins, 1991, 41:85-96), Okuda et al. (Prostaglandins, 1996, 52:375-384), and Koike et al. (FASEB J., 2003, 17:779-781). Evidence for the beneficial effect of an agonist of the PGI2 receptor in the treatment of diabetic nephropathy is given by Owada et al. (Nephron, 2002, 92:788-796) and Yamashita et al. (Diabetes Res. Clin. Pract., 2002, 57:149-161). Evidence for the beneficial effect of an agonist of the PGI2 receptor in the treatment of diabetic retinopathy is given by Yamagishi et al. (Mol. Med., 2002, 8:546-550), Burnette et al. (Exp. Eye Res., 2006, 83:1359-1365), and Hotta et al. (Diabetes, 1996, 45:361-366). It has been shown that an agonist of the PGI2 receptor can reduce increased tumor necrosis factor-α (TNF-α) levels in diabetic patients, implying that an agonist of the PGI2 receptor may contribute to the prevention of progression in diabetic complications (Fujiwara et al., Exp. Clin. Endocrinol. Diabetes, 2004, 112:390-394).

6. Glaucoma

Evidence that topical administration of an agonist of the PGI2 receptor can result in a decrease in intraocular pressure (IOP) in rabbits and dogs and thereby have beneficial effect in the treatment of glaucoma is given by Hoyng et al. (Hoyng et al., Invest. Ophthalmol. Vis. Sci., 1987, 28:470-476).

7. Hypertension

Agonists of the PGI2 receptor have been shown to have activity for regulation of vascular tone, for vasodilation, and for amelioration of pulmonary hypertension (see, e.g., Strauss et al., Clin Chest Med, 2007, 28:127-142; Driscoll et al., Expert Opin. Pharmacother., 2008, 9:65-81). Evidence for a beneficial effect of an agonist of the PGI2 receptor in the treatment of hypertension is given by Yamada et al. (Peptides, 2008, 29:412-418). Evidence that an agonist of the PGI2 receptor can protect against cerebral ischemia is given by Dogan et al. (Gen. Pharmacol., 1996, 27:1163-1166) and Fang et al. (J. Cereb. Blood Flow Metab., 2006, 26:491-501).

8. Anti-Inflammation Therapies

Anti-inflammation agents are prescribed for a variety of conditions. For example, in an inflammatory disease they are used to interfere with and thereby reduce an underlying deleterious There is evidence that a PGI2 receptor agonist can inhibit inflammation and thus be a potential treatment as an anti-inflammation therapy. It has been shown that an agonist of the PGI2 receptor can inhibit pro-inflammatory cytokine and chemokine (interleukin-12 (IL-12), tumor necrosis factor-α (TNF-α), IL-1α, IL-6, macrophage inflammatory protein-1alpha (MIP-1α), monocyte chemoattractant protein-1 (MCP-1)) production and T cell stimulatory function of dendritic cells (Jozefowski et al., Int. Immunopharmacol., 2003, 865-878; Thou et al., J. Immunol., 2007, 178:702-710; Nagao et al., Am. J. Respir. Cell Mol. Biol., 2003, 29:314-320; Idzko et al., J. Clin. Invest., 2007, 117:464-472). It has been shown that an agonist of the PGI2 receptor can inhibit pro-inflammatory cytokine (TNF-α, IL-1β, IL-6, granulocyte macrophage stimulating factor (GM-CSF)) production by macrophages (Raychaudhuri et al., J. Biol. Chem., 2002, 277:33344-33348; Czeslick et al., Eur. J. Clin. Invest., 2003, 33:1013-1017; Di Renzo et al., Prostaglandin Leukot. Essent. Fatty Acids, 2005, 73:405-410; Shinomiya et al., Biochem. Pharmacol., 2001, 61:1153-1160). It has been shown that an agonist of the PGI2 receptor can stimulate anti-inflammatory cytokine (IL-10) production by dendritic cells (Jozefowski et al., Int. Immunopharmacol., 2003, 865-878; Zhou et al., J. Immunol., 2007, 178:702-710). It has been shown that an agonist of the PGI2 receptor can stimulate anti-inflammatory cytokine (IL-10) production by macrophages (Shinomiya et al., Biochem. Pharmacol., 2001, 61:1153-1160). It has been shown that an agonist of the PGI2 receptor can inhibit a chemokine (CCL17)-induced chemotaxis of leukocytes (CD4⁺ Th2 T cells) (Jaffar et al., J. Immunol., 2007, 179:6193-6203). It has been shown that an agonist of the PGI2 receptor can confer protection from atherosclerosis, such as from atherothrombosis (Arehart et al., Curr. Med. Chem., 2007, 14:2161-2169; Stitham et al., Prostaglandins Other Lipid Mediat., 2007, 82:95-108; Fries et al., Hematology Am. Soc. Hematol. Educ. Program, 2005, :445-451; Egan et al., Science, 2004, 306:1954-1957; Kobayashi et al., J. Clin. Invest., 2004, 114:784-794; Arehart et al., Circ. Res., 2008, Mar. 6 Epub ahead of print). It has been shown that an agonist of the PGI2 receptor can attenuate asthma (Idzko et al., J. Clin. Invest., 2007, 117:464-472; Jaffar et al., J. Immunol., 2007, 179:6193-6203; Nagao et al., Am. J. Respir. Cell. Mol. Biol., 2003, 29:314-320). It has been shown that an agonist of the PGI2 receptor can decrease TNF-α production in type 2 diabetes patients (Fujiwara et al., Exp. Clin. Endocrinol. Diabetes, 2004, 112:390-394; Goya et al., Metabolism, 2003, 52:192-198). It has been shown that an agonist of the PGI2 receptor can inhibit ischemia-reperfusion injury (Xiao et al., Circulation, 2001, 104:2210-2215). It has been shown that an agonist of the PGI2 receptor can inhibit restenosis (Cheng et al., Science, 2002, 296:539-541). It has been shown that an agonist of the PGI2 receptor can attenuate pulmonary vascular injury and shock in a rat model of septic shock (Harada et al., Shock, 2008, Feb. 21 Epub ahead of print). It has been shown that an agonist of the PGI2 receptor can reduce the serum levels of TNF-α in vivo in patients with rheumatoid arthritis, and this is associated with improvement in the clinical course of the disease (Gao et al., Rheumatol. Int., 2002, 22:45-51; Boehme et al., Rheumatol. Int., 2006, 26:340-347).

The pharmaceutical compositions of the present invention disclosed herein provide beneficial reduction of inflammation. The pharmaceutical compositions of the present invention disclosed herein provide beneficial reduction of a deleterious inflammatory response associated with an inflammatory disease. Accordingly, in some embodiments, the present invention provides methods for reducing inflammation in a patient in need thereof, comprising administering to the patient a pharmaceutical composition comprising a PGI2 receptor agonist disclosed herein. In some embodiments, the present invention provides methods for decreasing IL-12, TNF-α, IL-1α, IL-10, IL-6, MIP-1α or MCP-1 production in a patient in need thereof, comprising administering to the patient a pharmaceutical composition comprising a PGI2 receptor agonist disclosed herein. In some embodiments, the present invention provides methods for decreasing TNF-α production in a patient in need thereof, comprising administering to the patient a pharmaceutical composition comprising a PGI2 receptor agonist disclosed herein. In some embodiments, the present invention provides methods for increasing IL-10 production in a patient in need thereof, comprising administering to the patient a pharmaceutical composition comprising a PGI2 receptor agonist disclosed herein. In some embodiments, the present invention provides methods for reducing a deleterious inflammatory response associated with an inflammatory disease in a patient in need thereof, comprising administering to the patient a pharmaceutical composition comprising a PGI2 receptor agonist disclosed herein. In some embodiments, the present invention provides methods for treating an inflammatory disease or a symptom thereof in a patient in need of the treatment comprising administering to the patient a pharmaceutical composition comprising a PGI2 receptor agonist disclosed herein. In some embodiments, the present invention provides methods for treating an inflammatory disease or a symptom thereof in a patient in need of the treatment comprising administering to the patient a pharmaceutical composition comprising a PGI2 receptor agonist disclosed herein. In some embodiments, the present invention provides methods for treating an inflammatory disease or a symptom thereof in a patient in need of the treatment comprising administering to the patient a pharmaceutical composition comprising a PGI2 receptor agonist disclosed herein, wherein the inflammatory disease is selected from the group consisting of psoriasis, psoriatic arthritis, rheumatoid arthritis, Crohn's disease, transplant rejection, multiple sclerosis, systemic lupus erythematosus (SLE), ulcerative colitis, ischemia-reperfusion injury, restenosis, atherosclerosis, acne, diabetes (including type 1 diabetes and type 2 diabetes), sepsis, chronic obstructive pulmonary disease (COPD), and asthma.

One aspect of the present invention relates to pharmaceutical compositions comprising an active pharmaceutical ingredient selected from: a salt as described herein, a solvate or hydrate of a salt as described herein, and a crystalline form as described herein; together with a pharmaceutically acceptable carrier.

One aspect of the present invention relates to methods of agonizing a PGI2 receptor by contacting the receptor with an active pharmaceutical ingredient of the present invention, or a pharmaceutical composition thereof.

One aspect of the present invention relates to methods for the treatment of a PGI2 receptor mediated disorder in an individual, comprising administering to said individual in need thereof, a therapeutically effective amount of an active pharmaceutical ingredient of the present invention, or a pharmaceutical composition thereof.

One aspect of the present invention relates to methods for the treatment of PAH in an individual, comprising administering to said individual in need thereof, a therapeutically effective amount of an active pharmaceutical ingredient of the present invention, or a pharmaceutical composition thereof.

One aspect of the present invention relates to methods for the treatment of idiopathic PAH in an individual, comprising administering to said individual in need thereof, a therapeutically effective amount of an active pharmaceutical ingredient of the present invention, or a pharmaceutical composition thereof.

One aspect of the present invention relates to methods for the treatment of familial PAH in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with a collagen vascular disease in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with a collagen vascular disease selected from: scleroderma, CREST syndrome, systemic lupus erythematosus (SLE), rheumatoid arthritis, Takayasu's arteritis, polymyositis, and dermatomyositis in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with a congenital heart disease in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with a congenital heart disease selected from: atrial septic defect (ASD), ventricular septic defect (VSD) and patent ductus arteriosus in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with portal hypertension in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with HIV infection in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with ingestion of a drug or toxin in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with hereditary hemorrhagic telangiectasia in an individual, comprising administering to said individual in need thereof, a therapeutically effective amount of a compound selected from: a salt of Compound I according to any one of an active pharmaceutical ingredient of the present invention, or a pharmaceutical composition thereof.

One aspect of the present invention relates to methods for the treatment of PAH associated with splenectomy in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with significant venous or capillary involvement in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with pulmonary veno-occlusive disease (PVOD) in an individual, comprising administering to said individual in need thereof, a therapeutically effective amount of an active pharmaceutical ingredient of the present invention, or a pharmaceutical composition thereof.

One aspect of the present invention relates to methods for the treatment of PAH associated with pulmonary capillary hemangiomatosis (PCH) in an individual, comprising administering to said individual in need thereof, a therapeutically effective amount of an active pharmaceutical ingredient of the present invention, or a pharmaceutical composition thereof, or a pharmaceutical composition thereof.

One aspect of the present invention relates to methods for the treatment of platelet aggregation in an individual, comprising administering to said individual in need thereof, a therapeutically effective amount of an active pharmaceutical ingredient of the present invention, or a pharmaceutical composition thereof.

One aspect of the present invention relates to methods for the treatment of: coronary artery disease, myocardial infarction, transient ischemic attack, angina, stroke, ischemia-reperfusion injury, restenosis or atrial fibrillation in an individual, comprising administering to said individual in need thereof, a therapeutically effective amount of an active pharmaceutical ingredient of the present invention, or a pharmaceutical composition thereof.

One aspect of the present invention relates to methods for reducing the risk of blood clot formation in an angioplasty or coronary bypass surgery individual comprising administering to said individual in need thereof, a therapeutically effective amount of an active pharmaceutical ingredient of the present invention, or a pharmaceutical composition thereof.

One aspect of the present invention relates to methods for reducing the risk of blood clot formation in an individual suffering from atrial fibrillation comprising administering to said individual in need thereof, a therapeutically effective amount of an active pharmaceutical ingredient of the present invention, or a pharmaceutical composition thereof.

One aspect of the present invention relates to methods for the treatment of atherosclerosis in an individual, comprising administering to said individual in need thereof, a therapeutically effective amount of an active pharmaceutical ingredient of the present invention, or a pharmaceutical composition thereof.

One aspect of the present invention relates to methods for the treatment of atherothrombosis in an individual, comprising administering to said individual in need thereof, a therapeutically effective amount of an active pharmaceutical ingredient of the present invention, or a pharmaceutical composition thereof.

One aspect of the present invention relates to methods for the treatment of asthma in an individual, comprising administering to said individual in need thereof, a therapeutically effective amount of an active pharmaceutical ingredient of the present invention, or a pharmaceutical composition thereof.

One aspect of the present invention relates to methods for the treatment of a symptom of asthma in an individual, comprising administering to said individual in need thereof, a therapeutically effective amount of an active pharmaceutical ingredient of the present invention, or a pharmaceutical composition thereof.

One aspect of the present invention relates to methods for the treatment of a diabetic-related disorder in an individual, comprising administering to said individual in need thereof, a therapeutically effective amount of an active pharmaceutical ingredient of the present invention, or a pharmaceutical composition thereof.

One aspect of the present invention relates to methods for the treatment of diabetic peripheral neuropathy in an individual, comprising administering to said individual in need thereof, a therapeutically effective amount of an active pharmaceutical ingredient of the present invention; or a pharmaceutical composition thereof.

One aspect of the present invention relates to methods for the treatment of diabetic nephropathy in an individual, comprising administering to said individual in need thereof, a therapeutically effective amount of an active pharmaceutical ingredient of the present invention, or a pharmaceutical composition thereof.

One aspect of the present invention relates to methods for the treatment of diabetic retinopathy in an individual, comprising administering to said individual in need thereof, a therapeutically effective amount of an active pharmaceutical ingredient of the present invention, or a pharmaceutical composition thereof.

One aspect of the present invention relates to methods for the treatment of glaucoma or other disease of the eye with abnormal intraocular pressure in an individual, comprising administering to said individual in need thereof, a therapeutically effective amount of an active pharmaceutical ingredient of the present invention, or a pharmaceutical composition thereof.

One aspect of the present invention relates to methods for the treatment of hypertension in an individual, comprising administering to said individual in need thereof, a therapeutically effective amount of an active pharmaceutical ingredient of the present invention, or a pharmaceutical composition thereof.

One aspect of the present invention relates to methods for the treatment of hypertension intended to confer protection against cerebral ischemia in an individual, comprising administering to said individual in need thereof, a therapeutically effective amount of an active pharmaceutical ingredient of the present invention, or a pharmaceutical composition thereof.

One aspect of the present invention relates to methods for the treatment of inflammation in an individual, comprising administering to said individual in need thereof, a therapeutically effective amount of an active pharmaceutical ingredient of the present invention, or a pharmaceutical composition thereof.

One aspect of the present invention relates to methods for the treatment of an inflammatory disease in an individual, comprising administering to said individual in need thereof, a therapeutically effective amount of an active pharmaceutical ingredient of the present invention, or a pharmaceutical composition thereof.

One aspect of the present invention relates to methods for the treatment of an inflammatory disease selected from: psoriasis, psoriatic arthritis, rheumatoid arthritis, Crohn's disease, transplant rejection, multiple sclerosis, systemic lupus erythematosus (SLE), ulcerative colitis, ischemia-reperfusion injury, restenosis, atherosclerosis, acne, type 1 diabetes, type 2 diabetes, sepsis, chronic obstructive pulmonary disorder (COPD) and asthma in an individual, comprising administering to said individual in need thereof, a therapeutically effective amount of an active pharmaceutical ingredient of the present invention, or a pharmaceutical composition thereof.

One aspect of the present invention relates to the use of an active pharmaceutical ingredient of the present invention in the manufacture of a medicament for the treatment of PAH.

One aspect of the present invention relates to the use of an active pharmaceutical ingredient of the present invention in the manufacture of a medicament for the treatment of idiopathic PAH.

Use an active pharmaceutical ingredient of the present invention, in the manufacture of a medicament for the treatment of familial PAH.

Use an active pharmaceutical ingredient of the present invention, in the manufacture of a medicament for the treatment of PAH associated with vascular collagen disease.

Use an active pharmaceutical ingredient of the present invention, in the manufacture of a medicament for the treatment of PAH associated with a collagen vascular disease selected from: scleroderma, CREST syndrome, systemic lupus erythematosus (SLE), rheumatoid arthritis, Takayasu's arteritis, polymyositis, and dermatomyositis.

Use an active pharmaceutical ingredient of the present invention, in the manufacture of a medicament for the treatment of PAH associated with a congenital heart disease.

Use an active pharmaceutical ingredient of the present invention, in the manufacture of a medicament for the treatment of PAH associated with a congenital heart disease selected from: atrial septic defect (ASD), ventricular septic defect (VSD) and patent ductus arteriosus.

One aspect of the present invention relates to the use of an active pharmaceutical ingredient of the present invention in the manufacture of a medicament for the treatment of PAH associated with portal hypertension.

One aspect of the present invention relates to the use of an active pharmaceutical ingredient of the present invention in the manufacture of a medicament for the treatment of PAH associated with HIV infection.

One aspect of the present invention relates to the use of an active pharmaceutical ingredient of the present invention in the manufacture of a medicament for the treatment of PAH associated with ingestion of a drug or toxin

One aspect of the present invention relates to the use of an active pharmaceutical ingredient of the present invention in the manufacture of a medicament for the treatment of PAH associated with hereditary hemorrhagic telangiectasia.

One aspect of the present invention relates to the use of an active pharmaceutical ingredient of the present invention in the manufacture of a medicament for the treatment of PAH associated with splenectomy.

One aspect of the present invention relates to the use of an active pharmaceutical ingredient of the present invention in the manufacture of a medicament for the treatment of PAH associated with significant venous or capillary involvement.

One aspect of the present invention relates to the use of an active pharmaceutical ingredient of the present invention in the manufacture of a medicament for the treatment of PAH associated with pulmonary veno-occlusive disease (PVOD).

One aspect of the present invention relates to the use of an active pharmaceutical ingredient of the present invention in the manufacture of a medicament for the treatment of PAH associated with pulmonary capillary hemangiomatosis (PCH).

One aspect of the present invention relates to the use of an active pharmaceutical ingredient of the present invention in the manufacture of a medicament for the treatment of platelet aggregation.

One aspect of the present invention relates to the use of an active pharmaceutical ingredient of the present invention in the manufacture of a medicament for the treatment of a PGI2 receptor mediated disorder selected from: coronary artery disease, myocardial infarction, transient ischemic attack, angina, stroke, ischemia-reperfusion injury, restenosis and atrial fibrillation.

One aspect of the present invention relates to the use of an active pharmaceutical ingredient of the present invention in the manufacture of a medicament for the treatment of blood clot formation in an angioplasty or coronary bypass surgery individual.

One aspect of the present invention relates to the use of an active pharmaceutical ingredient of the present invention in the manufacture of a medicament for the treatment of blood clot formation in an individual suffering from atrial fibrillation.

One aspect of the present invention relates to the use of an active pharmaceutical ingredient of the present invention in the manufacture of a medicament for the treatment of atherosclerosis.

One aspect of the present invention relates to the use of an active pharmaceutical ingredient of the present invention in the manufacture of a medicament for the treatment of atherothrombosis.

One aspect of the present invention relates to the use of an active pharmaceutical ingredient of the present invention in the manufacture of a medicament for the treatment of asthma.

One aspect of the present invention relates to the use of an active pharmaceutical ingredient of the present invention in the manufacture of a medicament for the treatment of a symptom of asthma.

One aspect of the present invention relates to the use of an active pharmaceutical ingredient of the present invention in the manufacture of a medicament for the treatment of a diabetic-related disorder.

One aspect of the present invention relates to the use of an active pharmaceutical ingredient of the present invention in the manufacture of a medicament for the treatment of diabetic peripheral neuropathy.

One aspect of the present invention relates to the use of an active pharmaceutical ingredient of the present invention in the manufacture of a medicament for the treatment of diabetic nephropathy.

One aspect of the present invention relates to the use of an active pharmaceutical ingredient of the present invention in the manufacture of a medicament for the treatment of diabetic retinopathy.

One aspect of the present invention relates to the use of an active pharmaceutical ingredient of the present invention in the manufacture of a medicament for the treatment of glaucoma or other disease of the eye with abnormal intraocular pressure.

One aspect of the present invention relates to the use of an active pharmaceutical ingredient of the present invention in the manufacture of a medicament for the treatment of hypertension.

One aspect of the present invention relates to the use of an active pharmaceutical ingredient of the present invention in the manufacture of a medicament for the treatment of hypertension intended to confer protection against cerebral ischemia.

One aspect of the present invention relates to the use of an active pharmaceutical ingredient of the present invention in the manufacture of a medicament for the treatment of inflammation.

One aspect of the present invention relates to the use of an active pharmaceutical ingredient of the present invention in the manufacture of a medicament for the treatment of an inflammatory disease.

One aspect of the present invention relates to the use of an active pharmaceutical ingredient of the present invention in the manufacture of a medicament for the treatment of an inflammatory disease selected from: psoriasis, psoriatic arthritis, rheumatoid arthritis, Crohn's disease, transplant rejection, multiple sclerosis, systemic lupus erythematosus (SLE), ulcerative colitis, ischemia-reperfusion injury, restenosis, atherosclerosis, acne, type 1 diabetes, type 2 diabetes, sepsis, chronic obstructive pulmonary disorder (COPD) and asthma.

One aspect of the present invention relates to the use of an active pharmaceutical ingredient of the present invention in the manufacture of a medicament for modulating the activity of a PGI2 receptor.

One aspect of the present invention relates to the use of an active pharmaceutical ingredient of the present invention in the manufacture of a medicament for agonizing a PGI2 receptor.

One aspect of the present invention relates to active pharmaceutical ingredients of the present invention for use in a method of treatment of a PGI2 receptor mediated disorder.

One aspect of the present invention relates to active pharmaceutical ingredients of the present invention for use in a method of treatment of PAH.

One aspect of the present invention relates to active pharmaceutical ingredients of the present invention for use in a method of treatment of idiopathic PAH.

One aspect of the present invention relates to active pharmaceutical ingredients of the present invention for use in a method of treatment of familial PAH.

One aspect of the present invention relates to active pharmaceutical ingredients of the present invention for use in a method of treatment of PAH associated with a collagen vascular disease.

One aspect of the present invention relates to active pharmaceutical ingredients of the present invention for use in a method of treatment of PAH associated with a collagen vascular disease selected from: scleroderma, CREST syndrome, systemic lupus erythematosus (SLE), rheumatoid arthritis, Takayasu's arteritis, polymyositis, and dermatomyositis.

One aspect of the present invention relates to active pharmaceutical ingredients of the present invention for use in a method of treatment of PAH associated with a congenital heart disease.

One aspect of the present invention relates to active pharmaceutical ingredients of the present invention for use in a method of treatment of PAH associated with a congenital heart disease selected from: atrial septic defect (ASD), ventricular septic defect (VSD) and patent ductus arteriosus.

One aspect of the present invention relates to active pharmaceutical ingredients of the present invention for use in a method of treatment of PAH associated with portal hypertension.

One aspect of the present invention relates to active pharmaceutical ingredients of the present invention for use in a method of treatment of PAH associated with HIV infection.

One aspect of the present invention relates to active pharmaceutical ingredients of the present invention for use in a method of treatment of PAH associated with ingestion of a drug or toxin.

One aspect of the present invention relates to active pharmaceutical ingredients of the present invention for use in a method of treatment of PAH associated with hereditary hemorrhagic telangiectasia.

One aspect of the present invention relates to active pharmaceutical ingredients of the present invention for use in a method of treatment of PAH associated with splenectomy.

One aspect of the present invention relates to active pharmaceutical ingredients of the present invention for use in a method of treatment of PAH associated with significant venous or capillary involvement.

One aspect of the present invention relates to active pharmaceutical ingredients of the present invention for use in a method of treatment of PAH associated with pulmonary veno-occlusive disease (PVOD).

One aspect of the present invention relates to active pharmaceutical ingredients of the present invention for use in a method of treatment of PAH associated with pulmonary capillary hemangiomatosis (PCH).

One aspect of the present invention relates to active pharmaceutical ingredients of the present invention for use in a method of treatment of platelet aggregation.

One aspect of the present invention relates to active pharmaceutical ingredients of the present invention for use in a method of treatment of: coronary artery disease, myocardial infarction, transient ischemic attack, angina, stroke, ischemia-reperfusion injury, restenosis or atrial fibrillation.

One aspect of the present invention relates to active pharmaceutical ingredients of the present invention for use in a method for the treatment of blood clot formation in an angioplasty or coronary bypass surgery individual.

One aspect of the present invention relates to active pharmaceutical ingredients of the present invention for use in a method for the treatment of blood clot formation in an individual suffering from atrial fibrillation.

One aspect of the present invention relates to active pharmaceutical ingredients of the present invention for use in a method of treatment of atherosclerosis.

One aspect of the present invention relates to active pharmaceutical ingredients of the present invention for use in a method of treatment of atherothrombosis.

One aspect of the present invention relates to active pharmaceutical ingredients of the present invention for use in a method of treatment of asthma.

One aspect of the present invention relates to active pharmaceutical ingredients of the present invention for use in a method of treatment of a symptom of asthma.

One aspect of the present invention relates to active pharmaceutical ingredients of the present invention for use in a method of treatment of a diabetic-related disorder.

One aspect of the present invention relates to active pharmaceutical ingredients of the present invention for use in a method of treatment of diabetic peripheral neuropathy.

One aspect of the present invention relates to active pharmaceutical ingredients of the present invention for use in a method of treatment of diabetic nephropathy.

One aspect of the present invention relates to active pharmaceutical ingredients of the present invention for use in a method of treatment of diabetic retinopathy.

One aspect of the present invention relates to active pharmaceutical ingredients of the present invention for use in a method of treatment of glaucoma or other disease of the eye with abnormal intraocular pressure.

One aspect of the present invention relates to active pharmaceutical ingredients of the present invention for use in a method of treatment of hypertension.

One aspect of the present invention relates to active pharmaceutical ingredients of the present invention for use in a method of treatment of hypertension intended to confer protection against cerebral ischemia.

One aspect of the present invention relates to active pharmaceutical ingredients of the present invention for use in a method of treatment of inflammation.

One aspect of the present invention relates to active pharmaceutical ingredients of the present invention for use in a method of treatment of an inflammatory disease. One aspect of the present invention relates to active pharmaceutical ingredients of the present invention for use in a method of treatment of an inflammatory disease selected from: psoriasis, psoriatic arthritis, rheumatoid arthritis, Crohn's disease, transplant rejection, multiple sclerosis, systemic lupus erythematosus (SLE), ulcerative colitis, ischemia-reperfusion injury, restenosis, atherosclerosis, acne, type 1 diabetes, type 2 diabetes, sepsis, chronic obstructive pulmonary disorder (COPD) and asthma.

One aspect of the present invention relates to active pharmaceutical ingredients of the present invention for use in a method of modulating the activity of a PGI2 receptor.

One aspect of the present invention relates to active pharmaceutical ingredients of the present invention for use in a method of agonizing a PGI2 receptor.

One aspect of the present invention relates to methods for the treatment of a PGI2 receptor mediated disorder in an individual, comprising administering to said individual in need thereof, a therapeutically effective amount of a salt, a solvate or hydrate of a salt, a crystalline, or a pharmaceutical composition of the present invention.

One aspect of the present invention relates to methods for the treatment of PAH in an individual, comprising administering to said individual in need thereof, a therapeutically effective amount of a salt, a solvate or hydrate of a salt, a crystalline, or a pharmaceutical composition of the present invention.

One aspect of the present invention relates to methods for the treatment of: idiopathic PAH; familial PAH; PAH associated with a collagen vascular disease selected from: scleroderma, CREST syndrome, systemic lupus erythematosus (SLE), rheumatoid arthritis, Takayasu's arteritis, polymyositis, and dermatomyositis; PAH associated with a congenital heart disease selected from: atrial septic defect (ASD), ventricular septic defect (VSD) and patent ductus arteriosus; PAH associated with portal hypertension; PAH associated with HIV infection; PAH associated with ingestion of a drug or toxin; PAH associated with hereditary hemorrhagic telangiectasia; PAH associated with splenectomy; PAH associated with significant venous or capillary involvement; PAH associated with pulmonary veno-occlusive disease (PVOD); or PAH associated with pulmonary capillary hemangiomatosis (PCH); in an individual, comprising administering to said individual in need thereof, a therapeutically effective amount of a salt, a solvate or hydrate of a salt, a crystalline, or a pharmaceutical composition of the present invention.

One aspect of the present invention relates to methods for the treatment of: platelet aggregation, coronary artery disease, myocardial infarction, transient ischemic attack, angina, stroke, ischemia-reperfusion injury, restenosis, atrial fibrillation, blood clot formation, atherosclerosis, atherothrombosis, asthma, a symptom of asthma, a diabetic-related disorder, diabetic peripheral neuropathy, diabetic nephropathy, diabetic retinopathy, glaucoma or other disease of the eye with abnormal intraocular pressure, hypertension, inflammation, an inflammatory disease, psoriasis, psoriatic arthritis, rheumatoid arthritis, Crohn's disease, transplant rejection, multiple sclerosis, systemic lupus erythematosus (SLE), ulcerative colitis, ischemia-reperfusion injury, restenosis, acne, type 1 diabetes, type 2 diabetes, sepsis, or chronic obstructive pulmonary disorder (COPD) in an individual, comprising administering to said individual in need thereof, a therapeutically effective amount of a salt, a solvate or hydrate of a salt, a crystalline, or a pharmaceutical composition of the present invention.

One aspect of the present invention relates to uses of a salt, a solvate or hydrate of a salt, or a crystalline form of the present invention, in the manufacture of a medicament for the treatment of a PGI2 receptor mediated disorder.

One aspect of the present invention relates to uses of a salt, a solvate or hydrate of a salt, or a crystalline form of the present invention, in the manufacture of a medicament for the treatment of PAH.

One aspect of the present invention relates to uses of a salt, a solvate or hydrate of a salt, or a crystalline form of the present invention, in the manufacture of a medicament for the treatment of: idiopathic PAH; familial PAH; PAH associated with vascular collagen disease selected from: scleroderma, CREST syndrome, systemic lupus erythematosus (SLE), rheumatoid arthritis, Takayasu's arteritis, polymyositis, and dermatomyositis; PAH associated with a congenital heart disease selected from: atrial septic defect (ASD), ventricular septic defect (VSD) and patent ductus arteriosus; PAH associated with portal hypertension; PAH associated with HIV infection; PAH associated with ingestion of a drug or toxin; PAH associated with hereditary hemorrhagic telangiectasia; PAH associated with splenectomy; PAH associated with significant venous or capillary involvement; PAH associated with pulmonary veno-occlusive disease (PVOD); or PAH associated with pulmonary capillary hemangiomatosis (PCH).

One aspect of the present invention relates to uses of a salt, a solvate or hydrate of a salt, or a crystalline form of the present invention, in the manufacture of a medicament for the treatment of: platelet aggregation, coronary artery disease, myocardial infarction, transient ischemic attack, angina, stroke, ischemia-reperfusion injury, restenosis, atrial fibrillation, blood clot formation, atherosclerosis, atherothrombosis, asthma, a symptom of asthma, a diabetic-related disorder, diabetic peripheral neuropathy, diabetic nephropathy, diabetic retinopathy, glaucoma or other disease of the eye with abnormal intraocular pressure, hypertension, inflammation, an inflammatory disease, psoriasis, psoriatic arthritis, rheumatoid arthritis, Crohn's disease, transplant rejection, multiple sclerosis, systemic lupus erythematosus (SLE), ulcerative colitis, ischemia-reperfusion injury, restenosis, acne, type 1 diabetes, type 2 diabetes, sepsis, or chronic obstructive pulmonary disorder (COPD).

One aspect of the present invention relates salts, solvates and hydrates of salts, crystalline forms, and pharmaceutical compositions of the present invention, for use in a method of treatment of the human or animal body by therapy.

One aspect of the present invention relates salts, solvates and hydrates of salts, crystalline forms, and pharmaceutical compositions of the present invention, for use in a method of treatment of a PGI2 receptor mediated disorder.

One aspect of the present invention relates salts, solvates and hydrates of salts, crystalline forms, and pharmaceutical compositions of the present invention, for use in a method of treatment of PAH.

One aspect of the present invention relates salts, solvates and hydrates of salts, crystalline forms, and pharmaceutical compositions of the present invention, for use in a method of treatment of: idiopathic PAH; familial PAH; PAH associated with vascular collagen disease selected from: scleroderma, CREST syndrome, systemic lupus erythematosus (SLE), rheumatoid arthritis, Takayasu's arteritis, polymyositis, and dermatomyositis; PAH associated with a congenital heart disease selected from: atrial septic defect (ASD), ventricular septic defect (VSD) and patent ductus arteriosus; PAH associated with portal hypertension; PAH associated with HIV infection; PAH associated with ingestion of a drug or toxin; PAH associated with hereditary hemorrhagic telangiectasia; PAH associated with splenectomy; PAH associated with significant venous or capillary involvement; PAH associated with pulmonary veno-occlusive disease (PVOD); or PAH associated with pulmonary capillary hemangiomatosis (PCH).

One aspect of the present invention relates salts, solvates and hydrates of salts, crystalline forms, and pharmaceutical compositions of the present invention, for use in a method of treatment of platelet aggregation, coronary artery disease, myocardial infarction, transient ischemic attack, angina, stroke, ischemia-reperfusion injury, restenosis, atrial fibrillation, blood clot formation, atherosclerosis, atherothrombosis, asthma, a symptom of asthma, a diabetic-related disorder, diabetic peripheral neuropathy, diabetic nephropathy, diabetic retinopathy, glaucoma or other disease of the eye with abnormal intraocular pressure, hypertension, inflammation, an inflammatory disease, psoriasis, psoriatic arthritis, rheumatoid arthritis, Crohn's disease, transplant rejection, multiple sclerosis, systemic lupus erythematosus (SLE), ulcerative colitis, ischemia-reperfusion injury, restenosis, acne, type 1 diabetes, type 2 diabetes, sepsis, or chronic obstructive pulmonary disorder (COPD).

### Pharmaceutical Compositions

One aspect of the present invention pertains to pharmaceutical compositions comprising a compound selected from: 2-(2-((4-(((4-chlorophenyl)(phenyl)carbamoyloxy)methyl)cyclohexyl)methoxy)acetamido)ethanesulfonic acid and pharmaceutically acceptable salts, solvates and hydrates thereof; and a pharmaceutically acceptable carrier, wherein said compound is prepared according to a process of the present invention.

One aspect of the present invention pertains methods of preparing a pharmaceutical composition, comprising admixing a compound selected from: 2-(2-((4-(((4-chlorophenyl)(phenyl)carbamoyloxy)methyl)cyclohexyl)methoxy)acetamido)ethanesulfonic acid and pharmaceutically acceptable salts, solvates and hydrates thereof and a pharmaceutically acceptable carrier, wherein said compound is prepared according to a process of the present invention.

One aspect of the present invention pertains to pharmaceutical compositions comprising an active pharmaceutical ingredient selected from: a salt of the present invention, a solvate or hydrate of a salt the present invention, and a crystalline form of the present invention; together with a pharmaceutically acceptable carrier.

One aspect of the present invention pertains to methods of preparing pharmaceutical compositions, comprising admixing an active pharmaceutical ingredient of the present invention together with a pharmaceutically acceptable carrier.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before filling and sealing an appropriate vial or ampule. These are just a few examples of the many appropriate methods well known in the art for preparing dosage forms.

An active pharmaceutical ingredient of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

While it is possible that, for use in the prophylaxis or treatment, an active pharmaceutical ingredient of the invention may, in an alternative use, be administered as a raw or pure chemical, it is preferable however to present the active pharmaceutical ingredient as a pharmaceutical formulation or composition further comprising a pharmaceutically acceptable carrier.

The invention thus further provides pharmaceutical formulations comprising an active pharmaceutical ingredient of the invention or a pharmaceutically acceptable salt, solvate, hydrate or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Brittan, Vol. 95, Marcel Dekker, Inc., New York, 1999, incorporated herein by reference in its entirety.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compositions of the invention, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compositions or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compositions of the present invention or a solvate, hydrate or physiologically functional derivative thereof can be used as active ingredients in pharmaceutical compositions, specifically as PGI2 receptor modulators. By the term “active ingredient” is defined in the context of a “pharmaceutical composition” and is intended to mean a component of a pharmaceutical composition that provides the primary pharmacological effect, as opposed to an “inactive ingredient” which would generally be recognized as providing no pharmaceutical benefit.

The dose when using the compositions of the present invention can vary within wide limits and as is customary and is known to the physician, it is to be tailored to the individual conditions in each individual case. It depends, for example, on the nature and severity of the illness to be treated, on the condition of the patient, on the active pharmaceutical ingredient employed or on whether an acute or chronic disease state is treated or prophylaxis is conducted or on whether further active compositions are administered in addition to the compositions of the present invention. Representative doses of the present invention include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular active pharmaceutical ingredient employed, whether a drug delivery system is utilized, on whether an acute or chronic disease state is being treated or prophylaxis is conducted or on whether further active compositions are administered in addition to the compositions of the present invention and as part of a drug combination. The dosage regimen for treating a disease condition with the compositions and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The compositions of the present invention can be administrated in a wide variety of oral and parenteral dosage forms. It will be obvious to those skilled in the art that the following dosage forms may comprise an active pharmaceutical ingredient of the invention.

For preparing pharmaceutical compositions from the compositions of the present invention, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size. The powders and tablets may contain varying percentage amounts of the active pharmaceutical ingredient. A representative amount in a powder or tablet may contain from 0.5 to about 90 percent of the active pharmaceutical ingredient; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” is intended to include the formulation of the active pharmaceutical ingredient with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compositions according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compositions of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the pharmaceutical compositions of the present invention as an aerosol can be prepared by processes well known to the person skilled in the art. For their preparation, for example, solutions or dispersions of the pharmaceutical compositions of the present invention in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the active pharmaceutical ingredient will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the active pharmaceutical ingredient in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The pharmaceutical compositions according to the invention may optionally comprise pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Certain pharmaceutical compositions of the present invention which contain a carboxylic acid functional group may optionally exist as pharmaceutically acceptable salts containing non-toxic, pharmaceutically acceptable metal cations and cations derived from organic bases. Representative metals include, but are not limited to, aluminium, calcium, lithium, magnesium, potassium, sodium, zinc and the like. In some embodiments the pharmaceutically acceptable metal is sodium. Representative organic bases include, but are not limited to, arginine, L-arginine, tris(trihydroxymethyl)aminomethane, benzathine (N¹,N²-dibenzylethane-1,2-diamine), chloroprocaine (2-(diethylamino)ethyl 4-(chloroamino)benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et al., Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The active pharmaceutical ingredients of this invention may form solvates with standard low molecular weight solvents using methods known to the skilled artisan.

Active pharmaceutical ingredients of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as active pharmaceutical ingredients of the invention containing one or more specialized non-toxic protective groups used in a transient manner to alter or to eliminate a property of the active pharmaceutical ingredient. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

The embodiments of the present invention include a method of producing a pharmaceutical composition for “combination-therapy” comprising admixing at least one active pharmaceutical ingredient according to any of the active pharmaceutical ingredient embodiments disclosed herein, together with at least one known pharmaceutical agent as described herein and a pharmaceutically acceptable carrier.

It is noted that when the PGI2 receptor modulators are utilized as active ingredients in a pharmaceutical composition, these are not intended for use only in humans, but in other non-human mammals as well. Indeed, recent advances in the area of animal health-care mandate that consideration be given for the use of active agents, such as PGI2 receptor modulators, for the treatment of an PGI2-associated disease or disorder in companionship animals (e.g., cats, dogs, etc.) and in livestock animals (e.g., cows, chickens, fish, etc.) Those of ordinary skill in the art are readily credited with understanding the utility of such active pharmaceutical ingredients in such settings.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

Illustrated syntheses of the present invention are shown Scheme I. The syntheses are further illustrated by the following examples. The following examples are provided to further define the invention without, however, limiting the invention to the particulars of these examples. The compounds and salts thereof described herein, supra and infra, are named according to the CS ChemDraw Ultra Version 7.0.1, AutoNom version 2.2, or CS ChemDraw Ultra Version 9.0.7. In certain instances common names are used and it is understood that these common names would be recognized by those skilled in the art.

Chemical shifts of proton nuclear magnetic resonance (¹H NMR) spectra are given in parts per million (ppm) with the residual solvent signal used as reference. NMR abbreviations are used as follows: s=singlet, d=doublet, t=triplet, q=quartet, m=multiplet, bs=broad singlet, bm=broad multiplet, bt=broad triplet.

LCMS spec: HPLC-pumps: LC-10AD VP, Shimadzu Inc.; HPLC system controller: SCL-10A VP, Shimadzu Inc; UV-Detector: SPD-10A VP, Shimadzu Inc; Autosampler: CTC HTS, PAL, Leap Scientific; Mass spectrometer: active pharmaceutical ingredient 150EX with Turbo Ion Spray source, AB/MDS Sciex; Software: Analyst 1.2.

### Example 1

### Preparation of Sodium 2-(((1r,4r)-4-(((4-Chlorophenyl)(phenyl)carbamoyloxy)methyl)cyclohexyl)methoxy)acetate

### Step A: Preparation of ((1r,4r)-4-(hydroxymethyl)cyclohexyl)methyl 4-chlorophenyl(phenyl)carbamate

4-Chloro-N-phenylaniline (15.0 g, 73.6 mmol), tribasic potassium phosphate, (fine powder, 4.69 g, 22.1 mmol), N,N-carbonyldiimidazole (13.14 g, 81 mmol) and acetonitrile (75 mL) were charged to a 500-mL, jacketed, four-necked cylindrical reaction flask equipped with a mechanical stirrer and a condenser. The reaction mixture was heated at 65° C. under nitrogen and monitored by HPLC. After about 2.5 h HPLC showed >98% conversion to the intermediate N-(4-chlorophenyl)-N-phenyl-1H-imidazole-1-carboxamide. After about 5.5 h a solution of (1r,4r)-cyclohexane-1,4-diyldimethanol (37.2 g, 258 mmol) in acetonitrile (150 mL) at 65° C. was added to the reaction mixture over 20 min. The resulting mixture was heated at 65° C. overnight. HPLC showed about 98% conversion to the required product. The mixture was filtered, and the cake was rinsed with acetonitrile (2×25 mL). The filtrate was concentrated under reduced pressure (40° C., 32 torr) 124.125 g of distillate was collected. The residue was diluted with water (50 mL) and this mixture was concentrated under reduced pressure (40° C., 32 torr) and 35.184 g of distillate was collected. The residue was diluted with water (50 mL) and the resulting mixture was allowed to stir overnight to give a white paste. The mixture was filtered, and the cake was rinsed with 25% acetonitrile/water (2×75 mL). The solid was dried in a vacuum oven to leave a white solid (22.271 g); 94.8% purity by HPLC peak area. LCMS m/z=374.3 [M+H]⁺; NMR (400 MHz, DMSO-d₆) S ppm 0.77-0.93 (m, 4H) 1.23 (dd, J=6.22, 3.51 Hz, 1H) 1.47 (dd, J=6.32, 2.91 Hz, 1H) 1.56-1.76 (m, 4H) 3.20 (t, J=5.78 Hz, 2H) 3.92 (d, J=6.13 Hz, 2H) 4.33 (t, J=5.31 Hz, 1H) 7.28-7.35 (m, 5H) 7.38-7.47 (m, 4H).

### Step B: Preparation of Sodium 2-(((1r,4r)-4-(((4-Chlorophenyl)(phenyl)carbamoyloxy)methyl)cyclohexyl)methoxy)acetate

In a 1 L 3-neck flask fitted with an overhead stirrer was placed ((1r,4r)-4-(hydroxymethyl)cyclohexyl)methyl 4-chlorophenyl(phenyl)carbamate (30 g), TBAB (7.8 g) and toluene (180 mL) and the mixture stirred at room temperature. To this mixture was added 50% NaOH (180 mL) followed by addition of tert-butyl bromoacetate (17.8 mL). The mixture was stirred at room temperature for 7 h. The mixture was then heated at 50-60° C. for 4 h. The mixture was then neutralized with concentrated HCl (300 mL). The mixture was filtered and the resulting filtrate was separated into two phases. The aqueous layer was extracted with toluene (80 mL). The combined organic layers were washed with water and the solvent was evaporated. The residue was azeotroped with isopropyl alcohol (150 mL) to remove the remaining toluene. Isopropyl alcohol (150 mL) was added to dissolve the residue and to this solution was added 12.5% NaOH solution (17 mL) to give a pH of 7-8. The resulting precipitate was collected by filtration and the filter cake was dissolved in water/acetone (280 mL; 1:1) at 55-60° C. The solution was filtered and the filtrate was diluted with acetone (320 mL) and stirred at room temperature overnight. The resulting slurry was cooled to 0-5° C. and then filtered. The filter cake was suspended in acetonitrile (400 mL), stirred at room temperature for 16 h and then filtered. The filter cake was dried at 60-70° C. under reduce pressure to leave the desired product (21.1 g); >99% purity by HPLC peak area. LCMS m/z=432.3 [M−Na+H]⁺.

### Example 2

### Preparation of Sodium 2-(((1r,4r)-4-(((4-Chlorophenyl)(phenyl)carbamoyloxy)methyl)cyclohexyl)methoxy)acetate

### Step A: Preparation of ((1r,4r)-4-(Hydroxymethyl)cyclohexyl)methyl 4-chlorophenyl(phenyl)carbamate

A 50-liter glass-lined reactor equipped with overhead agitation, jacket temperature control, and a nitrogen atmosphere was charged with (1r,4r)-cyclohexane-1,4-diyldimethanol (3.97 kg) and acetonitrile (12.71 kg). The reactor contents were stirred at 130 rpm and heated to 63° C. for 1.2 h to achieve dissolution. The mixture was cooled to <40° C. and then filtered. The filtrate was stored in a carboy. 4-Chloro-N-phenylaniline (1.60 kg), K₃PO₄ (0.50 kg), CDI (1.41 kg) and acetonitrile (6.29 kg) were charged to a 50-liter glass-lined reactor equipped with overhead agitation, jacket temperature control, and a nitrogen atmosphere. The reactor contents were stirred at 130 rpm and heated to 65° C. to 70° C. for 3 h, after which conversion of 4-chloro-N-phenylaniline to N-(4-chlorophenyl)-N-phenyl-1H-imidazole-1-carboxamide was 98.0% by HPLC peak area. The reaction mixture was cooled to less than 40° C. and the solution of (1r,4r)-cyclohexane-1,4-diyldimethanol in acetonitrile prepared earlier was added to the mixture. The reactor contents were stirred at 130 rpm and heated at 65 to 70° C. for 19 h, after which conversion of N-(4-chlorophenyl)-N-phenyl-1H-imidazole-1-carboxamide to ((1r,4r)-4-(hydroxymethyl)cyclohexyl)methyl 4-chlorophenyl(phenyl)carbamate was verified to be 98.0% by HPLC peak area. The reactor contents were filtered and the filter cake was rinsed with acetonitrile (2.00 kg). The filtrate was transferred back to the reactor and most of the acetonitrile (18.48 kg) was then removed at 22° C. by vacuum distillation at 80 mm Hg. Water (5.34 kg) was added to the reactor and 1.55 kg of water/acetonitrile mixture was then removed by vacuum distillation at 29° C. and 70 mm Hg. Water (5.34 kg) was added to the reactor and the product precipitated during the addition. The resulting mixture was stirred at 20° C. to 25° C. for 13 h. The precipitated product was filtered and washed with aqueous acetonitrile in two portions (1.59 kg acetonitrile dissolved in 6.00 kg water). The product was dried under reduced pressure at 60° C. (until loss-on-drying was 2 wt %) to give the title compound as an off-while solid (2.29 kg, 78% yield; 97% purity by HPLC peak area.)

### Step B: Preparation of Sodium 2-(((1r,4r)-4-(((4-Chlorophenyl)(phenyl)carbamoyloxy)methyl)cyclohexyl)methoxy)acetate

((1r,4r)-4-(Hydroxymethyl)cyclohexyl)methyl 4-chlorophenyl(phenyl)carbamate (1.70 kg), tetrabutylammonium bromide (0.44 kg) and toluene (7.36 kg) were charged to a 50-liter glass-lined reactor equipped with overhead agitation, jacket temperature control, and a nitrogen atmosphere. The mixture was stirred for 1 h at 20° C. To the resulting solution was added 50 wt % aqueous sodium hydroxide (15.34 kg) and the jacket temperature was set to 10° C. Then tert-butyl bromoacetate (1.33 kg) was added sufficiently slowly to maintain the stirred reaction mixture at 5-15° C. with reactor jacket cooling. The mixture was stirred at 5-15° C. for 8.1 h. Conversion of ((1r,4r)-4-(hydroxymethyl)cyclohexyl)methyl 4-chlorophenyl(phenyl)carbamate to tert-butyl 2-(((1r,4r)-4-(((4-chlorophenyl)(phenyl)carbamoyloxy)methyl)cyclohexyl)methoxy)acetate was >90.0% by HPLC peak area. The reactor contents were heated at 50-60° C. for 7.2 h. Conversion of tert-butyl (((1r,4r)-4-(((4-chlorophenyl)(phenyl)carbamoyloxy)methyl)cyclohexyl)methoxy)acetate to 2-(((1r,4r)-4-(((4-chlorophenyl)(phenyl)carbamoyloxy)methyl)cyclohexyl)methoxy)acetic acid was >90.0% by HPLC peak area. The reactor contents were then cooled to 15° C. and concentrated hydrochloric acid (18.87 kg) was added to the mixture at a rate sufficiently slow to maintain an internal temperature <50° C. The mixture was filtered to remove the solid sodium chloride from the reactor. The filtrate separated into two phases and the organic phase was removed. The aqueous layer was extracted with toluene (4.55 kg). The organic phases were combined and the mixture was distilled at 30° C. and 40 mm Hg to remove most of the toluene. Then, IPA (6.75 kg) was charged to the reactor and the resulting solution was distilled at 28° C. and 40 mm Hg to remove solvent (5.05 Kg). IPA (6.68 kg) was charged a second time to the reactor and the resulting mixture was vacuum distilled at 37° C. and 40 mm Hg to remove solvent (4.98 kg). Then, IPA (6.77 kg) was charged to the reactor for the third time and the reactor contents were heated to 40° C. Sodium hydroxide (12.5%, 0.87 kg) was added to the reactor. The resulting mixture had a pH of 7. The mixture was agitated at 155 rpm for 2 h at 40° C. The product precipitated, and the solid was filtered. The filter cake was washed with IPA (3.01 kg). The filter cake was transferred to a reactor using acetone (6.27 kg) and water (7.95 kg) and the mixture was heated at 59° C. for 3 h. The resulting mixture was filtered through a sintered glass filter and the filtrate was transferred to a reactor. Acetone (15.82 kg) was added and the mixture stirred for 66 h at 20° C. The reactor contents were further stirred at 0° C. for 2 hours, filtered and the filter cake was washed with acetone (3.2 kg). The filter cake was then transferred back to the reactor with the aid of acetonitrile (17.79 kg). The reactor contents were stirred at 100 rpm and 20° C. for 18.5 h. The slurry was filtered and the cake was washed with two portions of acetonitrile (10.26 kg total). The solid was dried at 65° C. to 70° C. under reduced pressure for 27 h, and then sieved through a 1.18 mm mesh screen. The product was further dried under reduced pressure at ≦70° C. to an acetonitrile level of ≦2000 ppm, to leave the title compound as a white to off-white solid (0.65 kg, 32% yield; 98.8% purity by HPLC peak area.)

### Example 3

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia)

Thionyl chloride (10 mL, 137 mmol) was added to sodium 2-(((1r,4r)-4-(((4-chlorophenyl)(phenyl)carbamoyloxy)methyl)cyclohexyl)methoxy)acetate (9.907 g, 21.83 mmol) with stirring and the resultant green mixture was stirred overnight at room temperature. The reaction mixture was concentrated and toluene (25 mL) was added. The mixture was concentrated under reduced pressure. More toluene (25 mL) was added and the mixture was concentrated to dryness under reduced pressure. The residue was taken up in THF (70 mL). The milky slurry of acid chloride in THF was slowly added to an ice-cold solution of 2-aminoethanesulfonic acid (10.93 g, 87 mmol) and sodium hydroxide (3.49 g, 87 mmol) in 40 mL of water. After the completion of the reaction, two phases were separated and the organic layer was washed with 10% NaOH solution (2×25 mL). The organic layer containing some particulates was dried with 1-2 g of Na₂SO₄ and filtered. The filtrate was acidified using concentrated HCl to pH 0-1. An aqueous layer (˜2 mL) separated and was discarded. The organic layer was concentrated to a syrup (27 g). Acetonitrile (50 mL) was added and precipitate formed. The slurry was concentrated and the residue was taken up in a mixture of acetonitrile (50 mL) and water (7.5 mL) and heated in a 50° C. bath. The resulting solution with some particulates was polish filtered using a Whatman #4 filter paper. To the clear filtrate (KF=5.7%) was added acetonitrile (25 mL) and the resulting mixture was cooled with stirring in an ice bath to give a slurry. The slurry was filtered and the filter-cake was washed with acetonitrile (20 mL). The wet cake was dried under reduced pressure at 65° C. overnight to give a white solid (8.2 g). Exact mass calculated for C₂₅H₃₁ClN₂O₇S: 538.2, found: LCMS m/z=539.4 [M+H]⁺; ¹H NMR (400 MHz, DMSO-d₆) S ppm 0.80-0.92 (bm, 4H), 1.36-1.51 (bm, 2H), 1.53-1.63 (bm, 2H), 1.65-1.76 (bm, 2H), 2.58 (t, J=6.5 Hz, 2H), 3.20 (d, J=6.4 Hz, 2H), 3.38 (q, J=6.2 Hz, 2H), 3.54 (s, H₂O—HOD peak), 3.76 (s, 2H), 3.89 (d, J=6.1 Hz, 2H), 7.24-7.31 (m, 5H), 7.36-7.43 (m, 4H), 7.90 (bt, J=5.4 Hz, 1H).

### Example 4

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Potassium Salt

2-Aminoethanesulfonic acid (2.90 g, 23.15 mmol) was dissolved in deionized water (40 mL) and 4-methylmorpholine (4.92 g, 48.6 mmol) and 1H-benzo[d][1,2,3]triazol-1-ol (3.55 g, 23.15 mmol) were added to the solution. 2-(((1r,4r)-4-(((4-chlorophenyl)(phenyl)carbamoyloxy)methyl)cyclohexyl)methoxy)acetic acid (10.00 g, 23.15 mmol) and THF (100 mL) were added followed by N1-((ethylimino)methylene)-N3,N3-dimethylpropane-1,3-diamine hydrochloride (4.88 g, 25.5 mmol) rinsing forward with deionized water (˜2 mL), THF (20 mL). The reaction mixture turned to clear pale yellowish solution and particulates were not visible. After stirring the reaction mixture overnight, more N1-((ethylimino)methylene)-N3,N3-dimethylpropane-1,3-diamine hydrochloride (0.100 g, 0.522 mmol) was added. After the reaction was complete, the mixture was cooled to 17° C. and solid KOH (1.299 g, 23.15 mmol) was added. The reaction mixture was concentrated on a rotary evaporator to 60-70 mL of a brownish syrup to which IPA (400 mL) was added. A slurry was obtained which was stirred at 20° C. overnight. The slurry was filtered using a Whatman filter cup to give 24.4 g of wet cake that was dried under reduced pressure at room temperature to give a solid (11.4 g) of solids.

Three lots of crude potassium 2-(2-(((1r,4r)-4-(((4-chlorophenyl)(phenyl)carbamoyloxy)methyl)cyclohexyl)methoxy)acetamido)ethanesulfonate (22.0 g, 38.1 mmol) were charged into a 3-necked 1-L flask fitted with a mechanical stirrer, a thermocouple, a condenser, and a nitrogen line. Deionized water (8.8 mL, 488 mmol) in acetone (100 mL) was added and the mixture was heated to 58° C. for 1-2 min. The slurry thinned but did not give a homogeneous solution. The thin slurry was cooled to 55° C. and acetone (340 mL) was added. The slurry was cooled to 38° C. and it turned to thick slurry with a somewhat gel-like appearance. Water (4.0 mL, 38.1 mmol) was added to form a white, granular, easily-stirred slurry that settled well upon stirring. The slurry was stirred at room temperature overnight and then filtered using a Whatman filter cup with a filter paper. The wet cake was pressed with a spatula to seal any cracks and washed with 2% water in acetone (44 mL). The wet cake (21.7 g) was dried under reduced pressure at room temperature to give a white solid (20.3 g). The water content was measured as 6.6 wt % by Karl Fischer titration, corresponding approximately to a dihydrate. Exact mass calculated for C₂₅H₃₁ClN₂O₇S: 538.2, found: LCMS m/z=539.4 [M+H]⁺; ¹H NMR (400 MHz, DMSO-d₆) δ ppm 0.80-0.99 (bm, 4H), 1.39-1.51 (bm, 2H), 1.54-1.59 (bm, 2H), 1.69-1.78 (bm, 2H), 2.54 (t, J=6.6 Hz, 2H), 3.20 (d, J=6.4 Hz, 2H), 3.30 (s, H₂O—HOD peak), 3.37 (q, J=6.8 Hz, 2H), 3.76 (s, 2H), 3.89 (d, J=6.1 Hz, 2H), 7.24-7.32 (m, 5H), 7.37-7.44 (m, 4H), 7.92 (bt, J=5.1 Hz, 1H).

### Example 5

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Potassium Salt Hydrate

2-Aminoethanesulfonic acid (0.038 g, 0.278 mmol) was dissolved in deionized water (0.4 mL) and 4-methylmorpholine (0.52 g, 0.51 mmol) was added to the solution. Then, 2-(((1r,4r)-4-(((4-chlorophenyl)(phenyl)carbamoyloxy)methyl)cyclohexyl)methoxy)acetic acid (0.102 g, 0.232 mmol) in THF (0.8 mL) was added to the reaction mixture. Chlorodimethoxytriazine (CDMT, 0.049 g, 0.278 mmol) was added to the mixture, rinsing forward with THF (0.4 mL). After the completion of reaction, the reaction mixture was treated with 2 N KOH in water (1 equivalent) and the mixture was concentrated. Isopropanol (4 mL) was added and the mixture was concentrated. Isopropanol (4 mL) was added to the residue and the mixture was stirred overnight, and then filtered to give the title compound (0.102 g) containing traces of taurine and CDMT-related impurities.

### Example 6

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Potassium Salt Hydrate

2-(2-(((1r,4r)-4-(((4-Chlorophenyl)(phenyl)carbamoyloxy)methyl)cyclohexyl)methoxy)acetamido)ethanesulfonic acid was dissolved in isopropanol with gentle heat, or ˜7% water in acetone at room temperature. To the free acid solution was added add 1 mole equivalent of potassium in the form of 2 N KOH solution. The mixture was slowly cooled and/or seeds of the title compound are added to crystallize the product. The title compound and was recovered by centrifuge filtration and allowed to air dry.

### Example 7

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Sodium Salt Hydrate II

2-(2-(((1r,4r)-4-(((4-Chlorophenyl)(phenyl)carbamoyloxy)methyl)cyclohexyl)methoxy)acetamido)ethanesulfonic acid (44.27 mg) was dissolved in hot isopropanol (1 mL). To this ˜60° C. solution, 2 N NaOH (0.045 mL) was added. The mixture was slowly cooled with stirring. After cooling to about 50° C., cloudiness began to show. The mixture was then allowed to cool to room temperature overnight to form the title compound as a crystalline precipitate, which was recovered by centrifuge filtration and allowed to air dry.

### Example 8

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Magnesium Salt Hydrate

To 2-(2-(((1r,4r)-4-(((4-chlorophenyl)(phenyl)carbamoyloxy)methyl)cyclohexyl)methoxy)acetamido)ethanesulfonic acid (52.81 mg) was added isopropanol (0.967 mL) and EtOH (0.483 mL) and the mixture heated to near clarity. A stoichiometric amount of Mg from a 1 M MgC12 solution (47 μL) was added and a small amount of precipitate formed immediately and remained after cooling to room temperature. The title compound precipitated overnight and it was recovered by centrifuge filtration and allowed to air dry.

### Example 9

### Preparation of 2-(2-(((1r,4r)-4-(((4-Chlorophenyl)(phenyl)carbamoyloxy)methyl)cyclohexyl)methoxy)acetamido)ethanesulfonic Acid (Compound I) Calcium Salt Hydrate I

A CaCl₂ counter ion solution was added to a hot suspension of sodium 2-(2-(((1r,4r)-4-(((4-chlorophenyl)(phenyl)carbamoyloxy)methyl)cyclohexyl)methoxy)acetamido)ethanesulfonate in isopropanol:ethanol (2:1) and the mixture was allowed to cool slowly forming a precipitate of the title compound, which was recovered by centrifuge filtration and allowed to air dry.

This title compound was also prepared by competition slurry with multiple Ca hydrate forms in EtOH at a water activity of 0.5.

### Example 10

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Calcium Salt Hydrate II

The title compound was prepared by adding 1.8 M CaCl₂ counter ion solution to a hot solution of 2-(2-(((1r,4r)-4-(((4-chlorophenyl)(phenyl)carbamoyloxy)methyl)cyclohexyl)methoxy)acetamido)ethanesulfonic acid, in isopropanol and ethanol (2:1) with about 3% water, and then slow cooling to form a precipitate, which was recovered by centrifuge filtration and allowed to air dry.

### Example 11

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Calcium Salt Hydrate III

A stoichiometric amount of a Ca(OAc)₂ counter ion solution was added to a suspension of sodium 2-(2-(((1r,4r)-4-(((4-chlorophenyl)(phenyl)carbamoyloxy)methyl)cyclohexyl)methoxy)acetamido)ethanesulfonate in hot isopropanol:water (2:1). No precipitate formed upon cooling. Partial evaporation of to about one-half volume produced a solid which was slurried overnight. The mixture was then allowed to stand overnight, and the title compound was recovered by centrifuge filtration and allowed to air dry.

### Example 12

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Calcium Salt Hydrate IV

To a hot (˜70° C.) suspension of 2-(2-(((1r,4r)-4-(((4-chlorophenyl)(phenyl)carbamoyloxy)methyl)cyclohexyl)methoxy)acetamido)ethanesulfonic acid, in THF was added 2 M Ca(OAc)₂ counter ion solution. The resulting clear hot solution (˜4% water) was cooled and seeds of hydrated Compound Ia calcium salt were added. No precipitate formed. The THF was removed by evaporation, isopropanol:water (96:4) was added followed by further seeds of hydrated Compound Ia calcium salt, and the title compound precipitated out and was recovered by centrifuge filtration and allowed to air dry.

### Example 13

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Tris(hydroxymethyl)aminomethane Salt (Compound Ia TRIS Salt)

The title compound was prepared by mixing a stoichiometric amount of 4 M aqueous tris(hydroxymethyl)aminomethane (TRIS) with 2-(2-(((1r,4r)-4-(((4-chlorophenyl)(phenyl)carbamoyloxy)methyl)cyclohexyl)methoxy)acetamido)ethanesulfonic acid in acetone with 5% water at ˜60° C. The total water content in the final crystallization solution was ˜10%. The title compound precipitated out on cooling and was recovered by centrifuge filtration and allowed to air dry.

### Example 14

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) Tris(hydroxymethyl)aminomethane Salt Hydrate (Compound Ia TRIS Salt Hydrate)

The title compound was prepared by slurrying the anhydrous TRIS salt of Compound Ia (Example 13) in water.

### Example 15

### Preparation of 2-(2-(((1r,4r)-4-(((4-Chlorophenyl)(phenyl)carbamoyloxy)methyl)cyclohexyl)methoxy)acetamido)ethanesulfonic Acid (Compound Ia) L-Arginine Salt Hydrate

The title compound was made by adding a stoichiometric amount of L-arginine as a 2.3 M aqueous solution to 2-(2-(((1r,4r)-4-(((4-chlorophenyl)(phenyl)carbamoyloxy)methyl)cyclohexyl)methoxy)acetamido)ethanesulfonic acid solution in acetone with 3% water at ˜50° C. The total water content in the final crystallization solution was ˜13%. The title compound precipitated out on cooling and was recovered by centrifuge filtration and allowed to air dry.

### Example 16

### Powder X-ray Diffraction

Powder X-ray Diffraction (PXRD) data were collected on an X′Pert PRO MPD powder diffractometer (PANalytical, Inc.; EQ0233) with a Cu source set at 45 kV and 40 mA, Cu(Kα) radiation and an X′Celerator detector. Samples were added to the sample holder and smoothed flat with a spatula and weigh paper. With the samples spinning, X-ray diffractograms were obtained by a 12-min scan over the 2-theta range 5-40°2θ Diffraction data were viewed and analyzed with the X′Pert Data Viewer Software, version 1.0a and X′Pert HighScore Software, version 1.0b.

### Example 17

### Differential Scanning Calorimetry

Differential scanning calorimetry (DSC) studies were conducted using a TA Instruments, Q2000 (EQ0090 or EQ1980) at heating rate 10° C./min from ˜25° C. to ˜220° C. The instruments were calibrated by the vendor for temperature and energy using the melting point and enthalpy of fusion of an indium standard. Thermal events (desolvation, melting, etc.) were evaluated using Universal Analysis 2000 software, version 4.1D, Build 4.1.0.16.

### Example 18

### Thermal Gravimetric Analysis

Thermogravimetric analyses (TGA) were conducted using a TA Instruments TGA Q500 (EQ0089) or Q5000 (EQ1982) at heating rate 10° C./min. The instruments were calibrated by the vendor using a standard weight for the balance, and Alumel and Nickel standards for the furnace (Curie point measurements). Thermal events such as weight-loss are calculated using the Universal Analysis 2000 software, version 4.1D, Build 4.1.0.16.

### Example 19

### Dynamic Moisture Sorption (DMS)

Samples are prepared for dynamic moisture-sorption analysis by placing ˜5 mg to ˜20 mg of compound in a tarred sample holder on the VTI balance. The instrument is a dynamic moisture-sorption analyzer, VTI Corporation, SGA-100, equipment #0228. A drying step is run at 40° C. and ˜1% RH for 1 h. The isotherm temperature is 25° C. A % weight change over 10 min (5 weight readings) of dm/dt=0.010 or 2 h, whichever occurs first, is required before continuing to the next step. The water content of the sample equilibrated as described above was determined from 30% RH to 90% RH and then back down to 10% RH.

Those skilled in the art will recognize that various modifications, additions, substitutions, and variations to the illustrative examples set forth herein can be made without departing from the spirit of the invention and are, therefore, considered within the scope of the invention. All documents referenced above, including, but not limited to, printed publications, and provisional and regular patent applications, are incorporated herein by reference in their entirety.

1.-54. (canceled)

55. A salt of a compound selected from: 2-(2-((4-(((4-chlorophenyl)(phenyl)carbamoyloxy)methyl)cyclohexyl)methoxy)acetamido)ethanesulfonic acid (Compound I): 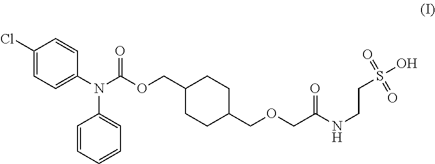
and solvates and hydrates thereof;
wherein the anion of said salt of Compound I is 2-(2-((4-(((4-chlorophenyl)(phenyl)carbamoyloxy)methyl)cyclohexyl)methoxy)acetamido)ethanesulfonate;
and wherein the cation of said salt of Compound I is selected from: potassium, calcium, magnesium, TRIS, and L-arginine.

56. The salt according to claim 55, wherein the cation is potassium.

57. The salt according to claim 55, which is a salt of 2-(2-((1r,4r)-4-(((4-chlorophenyl)(phenyl)carbamoyloxy)methyl)cyclohexyl)methoxy)acetamido)ethanesulfonic acid (Compound Ia):

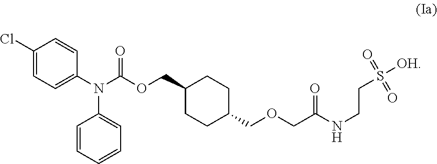

58. A solvate or hydrate of a salt according to claim 55, selected from the following solvates and hydrates:
potassium 2-(2-(((1r,4r)-4-(((4-chlorophenyl)(phenyl)carbamoyloxy)methyl)cyclohexyl)methoxy)acetamido)ethanesulfonate hydrate;
magnesium 2-(2-(((1r,4r)-4-(((4-chlorophenyl)(phenyl)carbamoyloxy)methyl)cyclohexyl)methoxy)acetamido)ethanesulfonate hydrate;
calcium 2-(2-(((1r,4r)-4-(((4-chlorophenyl)(phenyl)carbamoyloxy)methyl)cyclohexyl)methoxy)acetamido)ethanesulfonate hydrate I having an X-ray powder diffraction pattern comprising peaks, in terms of 2θ, at about 21.3°, about 20.7°, and about 18.3°;
calcium 2-(2-(((1r,4r)-4-(((4-chlorophenyl)(phenyl)carbamoyloxy)methyl)cyclohexyl)methoxy)acetamido)ethanesulfonate hydrate II having an X-ray powder diffraction pattern comprising peaks, in terms of 2θ, at about 23.5°, about 6.1°, and about 22.8°;
calcium 2-(2-(((1r,4r)-4-(((4-chlorophenyl)(phenyl)carbamoyloxy)methyl)cyclohexyl)methoxy)acetamido)ethanesulfonate hydrate III having an X-ray powder diffraction pattern comprising peaks, in terms of 2θ, at about 6.4°, about 11.4°, and about 13.4°;
calcium 2-(2-(((1r,4r)-4-(((4-chlorophenyl)(phenyl)carbamoyloxy)methyl)cyclohexyl)methoxy)acetamido)ethanesulfonate hydrate IV having an X-ray powder diffraction pattern comprising peaks, in terms of 2θ, at about 24.1°, about 10.2°, and about 21.0°;
TRIS 2-(2-(((1r,4r)-4-(((4-chlorophenyl)(phenyl)carbamoyloxy)methyl)cyclohexyl)methoxy)acetamido)ethanesulfonate hydrate; and
L-arginine 2-(2-(((1r,4r)-4-(((4-chlorophenyl)(phenyl)carbamoyloxy)methyl)cyclohexyl)methoxy)acetamido)ethanesulfonate hydrate.

59. A solvate or hydrate of a salt according to claim 58, which is:
potassium 2-(2-(((1r,4r)-4-(((4-chlorophenyl)(phenyl)carbamoyloxy)methyl)cyclohexyl)methoxy)acetamido)ethanesulfonate hydrate.

60. A crystalline form of a salt according to claim 55, selected from:
potassium 2-(2-(((1r,4r)-4-(((4-chlorophenyl)(phenyl)carbamoyloxy)methyl)cyclohexyl)methoxy)acetamido)ethanesulfonate hydrate;
magnesium 2-(2-(((1r,4r)-4-(((4-chlorophenyl)(phenyl)carbamoyloxy)methyl)cyclohexyl)methoxy)acetamido)ethanesulfonate hydrate;
calcium 2-(2-(((1r,4r)-4-(((4-chlorophenyl)(phenyl)carbamoyloxy)methyl)cyclohexyl)methoxy)acetamido)ethanesulfonate hydrate I having an X-ray powder diffraction pattern comprising peaks, in terms of 2θ, at about 21.3°, about 20.7°, and about 18.3°;
calcium 2-(2-(((1r,4r)-4-(((4-chlorophenyl)(phenyl)carbamoyloxy)methyl)cyclohexyl)methoxy)acetamido)ethanesulfonate hydrate II having an X-ray powder diffraction pattern comprising peaks, in terms of 2θ, at about 23.5°, about 6.1°, and about 22.8°;
calcium 2-(2-(((1r,4r)-4-(((4-chlorophenyl)(phenyl)carbamoyloxy)methyl)cyclohexyl)methoxy)acetamido)ethanesulfonate hydrate III having an X-ray powder diffraction pattern comprising peaks, in terms of 2θ, at about 6.4°, about 11.4°, and about 13.4°;
calcium 2-(2-(((1r,4r)-4-(((4-chlorophenyl)(phenyl)carbamoyloxy)methyl)cyclohexyl)methoxy)acetamido)ethanesulfonate hydrate IV having an X-ray powder diffraction pattern comprising peaks, in terms of 2θ, at about 24.1°, about 10.2°, and about 21.0°;
TRIS 2-(2-(((1r,4r)-4-(((4-chlorophenyl)(phenyl)carbamoyloxy)methyl)cyclohexyl)methoxy)acetamido)ethanesulfonate;
TRIS 2-(2-(((1r,4r)-4-(((4-chlorophenyl)(phenyl)carbamoyloxy)methyl)cyclohexyl)methoxy)acetamido)ethanesulfonate hydrate; and
L-arginine 2-(2-(((1r,4r)-4-(((4-chlorophenyl)(phenyl)carbamoyloxy)methyl)cyclohexyl)methoxy)acetamido)ethanesulfonate hydrate.

61. The crystalline form according to claim 60, wherein said compound is potassium 2-(2-(((1r,4r)-4-(((4-chlorophenyl)(phenyl)carbamoyloxy)methyl)cyclohexyl)methoxy)acetamido)ethanesulfonate hydrate.

62. The crystalline form according to claim 61 having an X-ray powder diffraction pattern substantially as shown in FIG. 8.

63. The crystalline form according to claim 61 having an X-ray powder diffraction pattern comprising peaks, in terms of 2θ, at about 9.8°, about 6.5°, and about 23.4°

64. The crystalline form according to claim 61 having a differential scanning calorimetry thermogram substantially as shown in FIG. 9.

65. The crystalline form according to claim 61 having a differential scanning calorimetry thermogram comprising an endotherm with an extrapolated onset temperature between about 135° C. and about 150° C.

66. The crystalline form according to claim 61 having a thermogravimetric analysis profile substantially as shown in FIG. 10

67. The crystalline form of claim 61 having a dynamic moisture sorption (DMS) profile substantially as shown in FIG. 11.

68. A pharmaceutical composition comprising an active pharmaceutical ingredient selected from: a salt according to claim 55; together with a pharmaceutically acceptable carrier.

69. A method of preparing a pharmaceutical composition according to claim 68, comprising admixing said active pharmaceutical ingredient together with a pharmaceutically acceptable carrier.

70. A method for the treatment of a PGI2 receptor mediated disorder in an individual, comprising administering to said individual in need thereof, a therapeutically effective amount of a salt according to claim 55.

71. A method for the treatment of PAH in an individual, comprising administering to said individual in need thereof, a therapeutically effective amount of a salt according to claim 55.

72. A method according to claim 71, wherein the PAH is:
idiopathic PAH;
familial PAH;
PAH associated with a collagen vascular disease selected from: scleroderma, CREST syndrome, systemic lupus erythematosus (SLE), rheumatoid arthritis, Takayasu's arteritis, polymyositis, and dermatomyositis;
PAH associated with a congenital heart disease selected from: atrial septic defect (ASD), ventricular septic defect (VSD) and patent ductus arteriosus;
PAH associated with portal hypertension;
PAH associated with HIV infection;
PAH associated with ingestion of a drug or toxin;
PAH associated with hereditary hemorrhagic telangiectasia;
PAH associated with splenectomy;
PAH associated with significant venous or capillary involvement;
PAH associated with pulmonary veno-occlusive disease (PVOD); or
PAH associated with pulmonary capillary hemangiomatosis (PCH).

73. A method for the treatment of: platelet aggregation, coronary artery disease, myocardial infarction, transient ischemic attack, angina, stroke, ischemia-reperfusion injury, restenosis, atrial fibrillation, blood clot formation, atherosclerosis, atherothrombosis, asthma, a symptom of asthma, a diabetic-related disorder, diabetic peripheral neuropathy, diabetic nephropathy, diabetic retinopathy, glaucoma or other disease of the eye with abnormal intraocular pressure, hypertension, inflammation, an inflammatory disease, psoriasis, psoriatic arthritis, rheumatoid arthritis, Crohn's disease, transplant rejection, multiple sclerosis, systemic lupus erythematosus (SLE), ulcerative colitis, ischemia-reperfusion injury, restenosis, acne, type 1 diabetes, type 2 diabetes, sepsis, or chronic obstructive pulmonary disorder (COPD) in an individual, comprising administering to said individual in need thereof, a therapeutically effective amount of a salt according claim 55.

74.-82. (canceled)

83. A salt of a compound selected from: 2-(2-((4-(((4-chlorophenyl)(phenyl)carbamoyloxy)methyl)cyclohexyl)methoxy)acetamido)ethanesulfonic acid (Compound I): 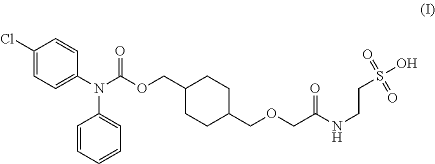
and solvates and hydrates thereof;
wherein the anion of said salt of Compound I is 2-(2-((4-(((4-chlorophenyl)(phenyl)carbamoyloxy)methyl)cyclohexyl)methoxy)acetamido)ethanesulfonate;
and wherein the cation of said salt of Compound I is sodium.

84. A solvate or hydrate of a salt according to claim 83, selected from the following solvates and hydrates:
sodium 2-(2-(((1r,4r)-4-(((4-chlorophenyl)(phenyl)carbamoyloxy)methyl)cyclohexyl)methoxy)acetamido)ethanesulfonate hydrate I having an X-ray powder diffraction pattern comprising peaks, in terms of 2θ, at about 6.4°, about 9.6°, and about 20.2°; and
sodium 2-(2-(((1r,4r)-4-(((4-chlorophenyl)(phenyl)carbamoyloxy)methyl)cyclohexyl)methoxy)acetamido)ethanesulfonate hydrate II having an X-ray powder diffraction pattern comprising peaks, in terms of 2θ, at about 19.6°, about 22.7°, and about 20.9°.

85. A crystalline form of a salt according to claim 83, selected from:
sodium 2-(2-(((1r,4r)-4-(((4-chlorophenyl)(phenyl)carbamoyloxy)methyl)cyclohexyl)methoxy)acetamido)ethanesulfonate hydrate I having an X-ray powder diffraction pattern comprising peaks, in terms of 2θ, at about 6.4°, about 9.6°, and about 20.2°; and
sodium 2-(2-(((1r,4r)-4-(((4-chlorophenyl)(phenyl)carbamoyloxy)methyl)cyclohexyl)methoxy)acetamido)ethanesulfonate hydrate II having an X-ray powder diffraction pattern comprising peaks, in terms of 2θ, at about 19.6°, about 22.7°, and about 20.9°.

86. A pharmaceutical composition comprising an active pharmaceutical ingredient selected from a salt according to claim 83 together with a pharmaceutically acceptable carrier.

87. A method of preparing a pharmaceutical composition according to claim 86, comprising admixing said active pharmaceutical ingredient together with a pharmaceutically acceptable carrier.

88. A method for the treatment of a PGI2 receptor mediated disorder in an individual, comprising administering to said individual in need thereof, a therapeutically effective amount of a salt according to claim 83.

89. A method for the treatment of PAH in an individual, comprising administering to said individual in need thereof, a therapeutically effective amount of a salt according to claim 83.

90. A method according to claim 89, wherein the PAH is:
idiopathic PAH;
familial PAH;
PAH associated with a collagen vascular disease selected from: scleroderma, CREST syndrome, systemic lupus erythematosus (SLE), rheumatoid arthritis, Takayasu's arteritis, polymyositis, and dermatomyositis;
PAH associated with a congenital heart disease selected from: atrial septic defect (ASD), ventricular septic defect (VSD) and patent ductus arteriosus;
PAH associated with portal hypertension;
PAH associated with HIV infection;
PAH associated with ingestion of a drug or toxin;
PAH associated with hereditary hemorrhagic telangiectasia;
PAH associated with splenectomy;
PAH associated with significant venous or capillary involvement;
PAH associated with pulmonary veno-occlusive disease (PVOD); or
PAH associated with pulmonary capillary hemangiomatosis (PCH).

91. A method for the treatment of: platelet aggregation, coronary artery disease, myocardial infarction, transient ischemic attack, angina, stroke, ischemia-reperfusion injury, restenosis, atrial fibrillation, blood clot formation, atherosclerosis, atherothrombosis, asthma, a symptom of asthma, a diabetic-related disorder, diabetic peripheral neuropathy, diabetic nephropathy, diabetic retinopathy, glaucoma or other disease of the eye with abnormal intraocular pressure, hypertension, inflammation, an inflammatory disease, psoriasis, psoriatic arthritis, rheumatoid arthritis, Crohn's disease, transplant rejection, multiple sclerosis, systemic lupus erythematosus (SLE), ulcerative colitis, ischemia-reperfusion injury, restenosis, acne, type 1 diabetes, type 2 diabetes, sepsis, or chronic obstructive pulmonary disorder (COPD) in an individual, comprising administering to said individual in need thereof, a therapeutically effective amount of a salt according claim 83.
